(19) United States
(12) Patent Application Publication
(10) Pub. No.: US 2022/0010300 A1
(43) Pub. Date: Jan. 13, 2022

(54) METHODS OF TREATING NEURODEGENERATIVE DISORDERS AND IDENTIFYING TARGETS THEREFORE

(71) Applicant: The Board of Trustees of the Leland Stanford Junior University, Stanford, CA (US)

(72) Inventors: Anton Wyss-Coray, Palo Alto (UD); Carolyn R. Bertozzi, Menlo Park, CA (US); Julia Marschallinger, Palo Alto, CA (US); Michael S. Haney, Mountain View, CA (US)

(21) Appl. No.: 17/413,360

(22) PCT Filed: Jan. 9, 2020
(86) PCT No.: PCT/US2020/012934

§ 371 Date: Jun. 11, 2021

Related U.S. Application Data
(60) Provisional application No. 62/791,355, filed on Jan. 11, 2019.

(51) Int. Cl. C12N 15/10; A61K 38/17; C07K 16/18; C12N 15/113

(52) U.S. Cl. CPC C12N 15/1037; A61K 38/1709; C12N 2310/14; C12N 15/113; C07K 2317/76; C07K 16/18

(57) ABSTRACT

Methods of treating a subject for a neurodegenerative disorder are provided. Aspects of the methods include administering to a subject in need thereof an effective amount of an agent that reduces the prevalence of lipid droplet accumulating microglia (LAM) to treat the subject for the neurodegenerative disorder. A variety of neurodegenerative disorders may be treated by practice of the methods. Also provided are methods of identifying lipid droplet-associated target genes, including target genes that are positive and negative regulators of lipid droplet formation, as well as methods of treating a neurodegenerative disorder in a subject by administering to the subject an antagonist of a positive regulator of lipid droplet formation and/or an agonist of a negative regulator of lipid droplet formation.

### CROSS-REFERENCE TO RELATED APPLICATIONS

This application claims the benefit of U.S. Provisional Patent Application No. 62/791,355, filed Jan. 11, 2019, which application is incorporated herein by reference in its entirety.

### GOVERNMENT RIGHTS

This invention was made with Government support under contract AG053015 awarded by the National Institutes of Health. The Government has certain rights in the invention.

### INTRODUCTION

Neurodegenerative diseases result from the deterioration of neurons, causing brain dysfunction. The diseases are loosely divided into two groups—conditions affecting memory that are ordinarily related to dementia and conditions causing problems with movements. The most widely known neurodegenerative diseases include Alzheimer (or Alzheimer's) disease and its precursor mild cognitive impairment (MCI), Parkinson's disease (including Parkinson's disease dementia), and multiple sclerosis.

As neurons deteriorate, an individual may first experience relatively mild symptoms—problems with coordination or remembering names. But as huge numbers of neurons die, symptoms progressively worsen. In some cases, patients lose the ability to walk independently, think clearly, or generally function in the world. Ultimately, many of these diseases are fatal.

According to current statistics, 5 million Americans suffer from Alzheimer's disease; 1 million from Parkinson's; 400,000 from multiple sclerosis (MS); 30,000 from amyotrophic lateral sclerosis (ALS or Lou Gehrig's disease), and 30,000 from Huntington's disease. In the United States in particular, because neurodegenerative diseases strike primarily in mid- to late-life, the incidence is expected to soar as the population ages. Current trends indicate that in 30 years, if such trends are left unaltered, more than 12 million Americans will suffer from neurodegenerative diseases.

Microglia are the resident immune cells of the central nervous system and play a pivotal role in the maintenance of brain homeostasis. In the aging brain and in neurodegeneration, microglia lose their homeostatic molecular signature and show profound functional impairments, such as increased production of pro-inflammatory cytokines, elevated generation of reactive oxygen species (ROS) and build-up of dysfunctional lysosomal deposits indicative of impaired phagocytosis. Microglia display high phenotypic diversity. Several microglia subpopulations and cellular states with unique transcriptional and functional signatures have been discovered and it has been suggested that these subsets respond differently to aging and disease.

### SUMMARY

Methods of treating a subject for a neurodegenerative disorder are provided. Aspects of the methods include administering to a subject in need thereof an effective amount of an agent that reduces the prevalence of lipid droplet accumulating microglia (LAM) to treat the subject for the neurodegenerative disorder. A variety of neurodegenerative disorders may be treated by practice of the methods. Also provided are methods of identifying lipid droplet-associated target genes, including target genes that are positive and negative regulators of lipid droplet formation, as well as methods of treating a neurodegenerative disorder in a subject by administering to the subject an antagonist of a positive regulator of lipid droplet formation and/or an agonist of a negative regulator of lipid droplet formation.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1J shows electron microscopy and confocal imaging of the accumulation of lipid droplets in microglia of mice.

FIG. 2A-2D shows confocal imaging of the accumulation of lipid droplets in the microglia in different brain regions of aged mice.

FIG. 3A-3H shows RNA-Seq analysis of lipid droplet low (LD-low) and LD-high microglia from aged mice.

FIG. 4A-4B shows lipidomic analysis of lipid droplets from the hippocampus and liver of 5- and 20-month old mice.

FIG. 5A-5F shows the effect of lipopolysaccharide (LPS) treatment on lipid droplet formation in microglia.

FIG. 6A-6J shows the relationship between lipid droplet accumulating microglia (LAM) and lipid droplets in BV-2 cells with impaired phagocytosis.

FIG. 7A-7K shows that LAM and lipid droplet-rich BV-2 cells show increased ROS production and LAM secrete elevated levels of inflammatory cytokines.

FIG. 8A-8M shows a CRISPR-Cas9 screen that identifies genetic regulators of lipid droplet formation.

FIG. 9A-9F provides the transcriptional signature of LAM.

FIG. 10A-10B shows signs of metabolic alterations in LAM.

### DEFINITIONS

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with cancer; those suspected of having cancer; etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as CH). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction;

(4) F(ab)₂ is a dimer of two Fab′ fragments held together by two disulfide bonds;

(5) Fv, defined as a genetically engineered fragment containing the variable region of the light chain and the variable region of the heavy chain expressed as two chains;

(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and

(7) “3G”, including single domain (typically a variable heavy domain devoid of a light chain) and “miniaturized” antibody molecules (typically a full-sized Ab or mAb in which non-essential domains have been removed).

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes lipoproteins, glycoproteins, and the like.

A “host cell,” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector, a guide RNA, a donor DNA template, and the like. For example, a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

### DETAILED DESCRIPTION

Methods of treating a subject for a neurodegenerative disorder are provided. Aspects of the methods include administering to a subject in need thereof an effective amount of an agent that reduces the prevalence of lipid droplet accumulating microglia (LAM) to treat the subject for the neurodegenerative disorder. A variety of neurodegenerative disorders may be treated by practice of the methods. Also provided are methods of identifying lipid droplet-associated target genes, including target genes that are positive and negative regulators of lipid droplet formation, as well as methods of treating a neurodegenerative disorder in a subject by administering to the subject an antagonist of a positive regulator of lipid droplet formation and/or an agonist of a negative regulator of lipid droplet formation.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, aspects of the instant disclosure include methods of treating a subject for a neurodegenerative disorder. A variety of neurodegenerative disorders may be treated by practice of the methods described herein.

Neurodegenerative disorders are defined as hereditary and sporadic conditions that are characterized by progressive nervous system dysfunction. These disorders are often associated with atrophy of the affected central or peripheral structures of the nervous system. Neurodegenerative disorders include, but are not limited to e.g., diseases such as Alzheimer's Disease and other dementias, Brain Cancer, Degenerative Nerve Diseases, Encephalitis,

Epilepsy, Genetic Brain Disorders, Head and Brain Malformations, Hydrocephalus, Stroke, Parkinson's Disease, Multiple Sclerosis, Amyotrophic Lateral Sclerosis (ALS or Lou Gehrig's Disease), Huntington's Disease, Prion Diseases, and others.

Alzheimer's disease is a progressive, inexorable loss of cognitive function associated with an excessive number of senile plaques in the cerebral cortex and subcortical gray matter, which also contains b-amyloid and neurofibrillary tangles consisting of tau protein. The common form affects persons >60 yr old, and its incidence increases as age advances. It accounts for more than 65% of the dementias in the elderly.

The cause of Alzheimer's disease is not known. The disease runs in families in about 15 to 20% of cases. The remaining, so-called sporadic cases have some genetic determinants. The disease has an autosomal dominant genetic pattern in most early-onset and some late-onset cases but a variable late-life penetrance. Environmental factors are the focus of active investigation.

In the course of the disease, synapses, and ultimately neurons are lost within the cerebral cortex, hippocampus, and subcortical structures (including selective cell loss in the nucleus basalis of Meynert), locus caeruleus, and nucleus raphae dorsalis. Cerebral glucose use and perfusion is reduced in some areas of the brain (parietal lobe and temporal cortices in early-stage disease, prefrontal cortex in late-stage disease). Neuritic or senile plaques (composed of neurites, astrocytes, and glial cells around an amyloid core) and neurofibrillary tangles (composed of paired helical filaments) play a role in the pathogenesis of Alzheimer's disease. Senile plaques and neurofibrillary tangles occur with normal aging, but they are much more prevalent in persons with Alzheimer's disease.

Parkinson's Disease (PD) is an idiopathic, slowly progressive, degenerative CNS disorder characterized by slow and decreased movement, muscular rigidity, resting tremor, and postural instability. Originally considered primarily a motor disorder, PD is now recognized to also affect cognition, behavior, sleep, autonomic function, and sensory function. The most common cognitive impairments include an impairment in attention and concentration, working memory, executive function, producing language, and visuospatial function.

In primary Parkinson's disease, the pigmented neurons of the substantia nigra, locus caeruleus, and other brain stem dopaminergic cell groups are lost. The cause is not known. The loss of substantia nigra neurons, which project to the caudate nucleus and putamen, results in depletion of the neurotransmitter dopamine in these areas. Onset is generally after age 40, with increasing incidence in older age groups.

Secondary parkinsonism results from loss of or interference with the action of dopamine in the basal ganglia due to other idiopathic degenerative diseases, drugs, or exogenous toxins. The most common cause of secondary parkinsonism is ingestion of antipsychotic drugs or reserpine, which produce parkinsonism by blocking dopamine receptors. Less common causes include carbon monoxide or manganese poisoning, hydrocephalus, structural lesions (tumors, infarcts affecting the midbrain or basal ganglia), subdural hematoma, and degenerative disorders, including striatonigral degeneration.

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. Symptoms are classified into three groups based on the functions of the frontal and temporal lobes affected: Behavioural variant FTD (bvFTD), with symptoms include lethargy and aspontaneity on the one hand, and disinhibition on the other; progressive nonfluent aphasia (PNFA), in which a breakdown in speech fluency due to articulation difficulty, phonological and/or syntactic errors is observed but word comprehension is preserved; and semantic dementia (SD), in which patients remain fluent with normal phonology and syntax but have increasing difficulty with naming and word comprehension. Other cognitive symptoms common to all FTD patients include an impairment in executive function and ability to focus. Other cognitive abilities, including perception, spatial skills, memory and praxis typically remain intact. FTD can be diagnosed by observation of reveal frontal lobe and/or anterior temporal lobe atrophy in structural MRI scans.

A number of forms of FTD exist. For example, one form of frontotemporal dementia is Semantic Dementia (SD). SD is characterized by a loss of semantic memory in both the verbal and non-verbal domains. SD patients often present with the complaint of word-finding difficulties. Clinical signs include fluent aphasia, anomia, impaired comprehension of word meaning, and associative visual agnosia (the inability to match semantically related pictures or objects). As the disease progresses, behavioral and personality changes are often seen similar to those seen in frontotemporal dementia although cases have been described of ‘pure’ semantic dementia with few late behavioral symptoms. Structural MRI imaging shows a characteristic pattern of atrophy in the temporal lobes (predominantly on the left), with inferior greater than superior involvement and anterior temporal lobe atrophy greater than posterior.

As another example, another form of frontotemporal dementia is Pick's disease (PiD, also PcD). A defining characteristic of the disease is build-up of tau proteins in neurons, accumulating into silver-staining, spherical aggregations known as “Pick bodies”. Symptoms include loss of speech (aphasia) and dementia. Patients with orbitofrontal dysfunction can become aggressive and socially inappropriate. They may steal or demonstrate obsessive or repetitive stereotyped behaviors. Patients with dorsomedial or dorsolateral frontal dysfunction may demonstrate a lack of concern, apathy, or decreased spontaneity. Patients can demonstrate an absence of self-monitoring, abnormal self-awareness, and an inability to appreciate meaning. Patients with gray matter loss in the bilateral posterolateral orbitofrontal cortex and right anterior insula may demonstrate changes in eating behaviors, such as a pathologic sweet tooth. Patients with more focal gray matter loss in the anterolateral orbitofrontal cortex may develop hyperphagia. While some of the symptoms can initially be alleviated, the disease progresses and patients often die within two to ten years.

Huntington's disease (HD) is a hereditary progressive neurodegenerative disorder characterized by the development of emotional, behavioral, and psychiatric abnormalities; loss of intellectual or cognitive functioning; and movement abnormalities (motor disturbances). The classic signs of HD include the development of chorea—involuntary, rapid, irregular, jerky movements that may affect the face, arms, legs, or trunk—as well as cognitive decline including the gradual loss of thought processing and acquired intellectual abilities. There may be impairment of memory, abstract thinking, and judgment; improper perceptions of time, place, or identity (disorientation); increased agitation; and personality changes (personality disintegration). Although symptoms typically become evident during the fourth or fifth decades of life, the age at onset is variable and ranges from early childhood to late adulthood (e.g., 70s or 80s).

HD is transmitted within families as an autosomal dominant trait. The disorder occurs as the result of abnormally long sequences or “repeats” of coded instructions within a gene on chromosome 4 (4p16.3). The progressive loss of nervous system function associated with HD results from loss of neurons in certain areas of the brain, including the basal ganglia and cerebral cortex.

Amyotrophic lateral sclerosis (ALS) is a rapidly progressive, invariably fatal neurological disease that attacks motor neurons. Muscular weakness and atrophy and signs of anterior horn cell dysfunction are initially noted most often in the hands and less often in the feet. The site of onset is random, and progression is asymmetric. Cramps are common and may precede weakness. Rarely, a patient survives 30 years; 50% die within 3 years of onset, 20% live 5 years, and 10% live 10 years. Diagnostic features include onset during middle or late adult life and progressive, generalized motor involvement without sensory abnormalities. Nerve conduction velocities are normal until late in the disease. Recent studies have documented the presentation of cognitive impairments as well, particularly a reduction in immediate verbal memory, visual memory, language, and executive function.

A decrease in cell body area, number of synapses and total synaptic length has been reported in even normal-appearing neurons of the ALS patients. It has been suggested that when the plasticity of the active zone reaches its limit, a continuing loss of synapses can lead to functional impairment. Promoting the formation or new synapses or preventing synapse loss may maintain neuron function in these patients.

Multiple Sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Common cognitive impairments include impairments in memory (acquiring, retaining, and retrieving new information), attention and concentration (particularly divided attention), information processing, executive functions, visuospatial functions, and verbal fluency. Common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive.

In some instances, the neurodegenerative disorder treated according to the methods as described herein may be a neurodegenerative disorder selected from Alzheimer's disease, Parkinson's disease, Amyotrophic Lateral Sclerosis, Huntington's disease, and frontotemporal dementia.

By “treatment” it is meant that at least an amelioration of one or more symptoms associated with a neurodegenerative disorder afflicting the subject is achieved, where amelioration is used in a broad sense to refer to at least a reduction in the magnitude of a parameter, e.g., a symptom associated with the impairment being treated. As such, treatment also includes situations where a pathological condition, or at least symptoms associated therewith, are completely inhibited, e.g., prevented from happening, or stopped, e.g., terminated, such that the adult mammal no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, increased neurogenesis, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

As summarized above, methods of treating a subject for a neurodegenerative disorder may include administering to a subject in need thereof an effective amount of an agent that reduces the prevalence of lipid droplet accumulating microglia (LAM). Without being bound by theory, the instant disclosure includes the discovery of a subpopulation or state of microglia in the aging brain which accumulate lipid droplets, termed LAM. The discovered LAM exhibit a unique transcriptional signature, show defects in phagocytosis, produce increased levels of ROS and release elevated levels of pro-inflammatory cytokines. LAM represent a dysfunctional and pro-inflammatory microglia state in the aging brain. Accordingly, the presence of LAM is associated with the presence and/or progression of neurodegenerative disorders and methods of the present disclosure include targeting LAM to treat neurodegenerative disorders. Neurodegenerative disorders that may be targeted by the herein disclosed methods include essentially any disorder where lipid droplet accumulation and/or the presence of LAM is associated with the presence and/or progression of the neurodegenerative disorder.

Methods of targeting lipid droplet formation and/or LAM to treat a neurodegenerative disorder will vary. In some instances, methods of the present disclosure may include targeting a gene associated with lipid droplet formation, including positive and negative regulators of lipid droplet formation. For example, in some instances, a negative regulator of lipid droplet formation may be targeted with an agonist of the negative regulator. In some instances, a positive regulator of lipid droplet formation may be targeted with an antagonist of the positive regulator. In some instances, LAM may be directly targeted, including e.g., where LAM are targeted for ablation or the growth/proliferation of LAM is directly or indirectly targeted. In some instances, lipid droplet accumulation in LAM is directly targeted.

Various subjects may be treated in the methods of the present disclosure. In some instances, treated subjects may be mammals, including but not limited to e.g., rodents (e.g., rats, mice, etc.), non-human primates (e.g., macaques, marmosets, tamarins, spider monkeys, owl monkeys, vervet monkeys, squirrel monkeys, baboons, chimpanzees, etc.), humans, and the like. In some instances, a treated subject may be an animal model (e.g., a rodent model, a non-human primate model, etc.) of a neurodegenerative disorder. In some instances, a treated subject may be a human subject, including but not limited to e.g., a human subject having a neurodegenerative disorder, a human subject at increased risk of developing a neurodegenerative disorder, a human subject of advanced age (e.g., at least 60 years of age, at least 65 years of age, at least 70 years of age, at least 75 years of age, at least 80 years of age, at least 85 years of age, at least 90 years of age, etc.), or a combination thereof. Treated subjects may or may not be symptomatic, e.g., subject may or may not display or have previously displayed one or more symptoms of a neurodegenerative disorder, including but not limited to e.g., those neurodegenerative disorders described herein.

Methods of the present disclosure may include administering to a subject an agent that reduces the prevalence of LAM. Useful agents for reducing the prevalence of LAM include agents that modulate lipogenesis, agents that modulate lipolysis, agents that modulate lipid droplet formation, agents that modulate genetic regulators of lipid droplet formation (e.g., including microglial lipid droplet formation), combinations thereof and the like. Useful agents include agonists and antagonists, including e.g., agonists of negative regulators of microglial lipid droplet formation and antagonists of positive regulators of microglial lipid droplet formation.

Agents that modulate lipogenesis may negatively or positively influence the metabolic formation of fat. For example, agents that inhibit lipogenesis may prevent the metabolic formation of fat and thus inhibit the formation of lipid droplet and the accumulation of lipid droplets in LAM. Agents that modulate lipolysis may negatively or positively influence the breakdown of fats and other lipids, e.g., by hydrolysis to release fatty acids. For example, agents that promote lipolysis may increase the breakdown of fats and other lipids and thus inhibit the formation of lipid droplet and the accumulation of lipid droplets in LAM. In some instances, a target gene may be involved in lipogenesis (i.e., a lipogenesis gene) and inhibition of the function of the target gene may be performed by administering to the subject an antagonist of the target gene that results in reduced lipogenesis. In some instances, a target gene may be involved in lipolysis (i.e., a lipolysis gene) and promotion of the function of the target gene may be performed by administering to the subject an agonist of the target gene that results in increased lipolysis.

As agonists, any useful inducer of the subject target gene and/or encoded product thereof may be employed in the subject methods. Non-limiting examples of useful inducers include but are not limited to e.g., non-peptide small molecule agonists, peptide agonists, encoding nucleic acids, and the like. In some instances, useful agonists may include mimetics. In some instances, inducers may include the protein (including recombinant and modified forms thereof), or a peptide fragment thereof (including recombinant and modified forms thereof), or a nucleic acid encoding the protein including where the protein shares 100% sequence identity or less than 100% sequence identity, including e.g., at least 99%, at least 98%, at least 97% at least 96%, at least 95%, at least 90%, at least 85%, at least 80%, etc., sequence identity, with a protein or amino acid sequence of a protein described herein. In some instances, inducers may include a nucleic acid encoding the protein, or a fragment thereof, including where the nucleic acid shares 100% sequence identity or less than 100% sequence identity, including e.g., at least 99%, at least 98%, at least 97% at least 96%, at least 95%, at least 90%, at least 85%, at least 80%, etc., sequence identity, with an encoding nucleic acid identified herein. Accordingly, as non-limiting examples, in some instances, useful inducers may include a non-peptide small molecule agonist of a protein that is a negative regulator of microglial lipid droplet formation, a peptide agonist of a protein that is a negative regulator of microglial lipid droplet formation, a RNA or DNA encoding a protein (or fragment thereof) that is a negative regulator of microglial lipid droplet formation, and the like. In some instances, the effectiveness of an inducer may be confirmed using an in vitro or in vivo assay, including e.g., where the effectiveness of the inducer is compared to an appropriate control or standard, e.g., a corresponding CRISPR-based gene silencing agent, a conventional therapy, etc. In some instances, useful agonists may be identified by screening candidate compounds, including but not limited to e.g., where libraries of compounds (e.g., small molecule chemical libraries) are screened in a high throughput manner.

As antagonists, any useful inhibitor of the subject target gene and/or encoded product thereof may be employed in the subject methods. Non-limiting examples of useful inhibitors include but are not limited to e.g., non-peptide small molecule antagonists, peptide antagonists, interfering RNAs (e.g., siRNA, shRNA, etc.), antibodies (e.g., neutralizing antibodies, function blocking antibodies, etc.), aptamers, and the like. In some instances, inhibitors may target, e.g., specifically bind to, specifically hybridize to, etc., a target protein or a nucleic acid encoding a target protein including where the protein shares 100% sequence identity or less than 100% sequence identity, including e.g., at least 99%, at least 98%, at least 97% at least 96%, at least 95%, at least 90%, at least 85%, at least 80%, etc., sequence identity, with a protein or amino acid sequence of a protein described herein.

Accordingly, as non-limiting examples, in some instances, useful inhibitors may include a non-peptide small molecule antagonist of a protein that is a positive regulator of microglial lipid droplet formation, a peptide antagonist of a protein that is a positive regulator of microglial lipid droplet formation, an interfering RNA targeting an RNA expressed from a gene that positively regulates microglial lipid droplet formation, an anti-lipid-droplet-promoting-protein antibody (e.g., an antibody that specifically binds to a lipid-droplet-promoting protein), an anti-lipid-droplet-promoting-protein aptamer, and the like. In some instances, the effectiveness of an inhibitor may be confirmed using an in vitro or in vivo assay, including e.g., where the effectiveness of the inhibitor is compared to an appropriate control or standard, e.g., the corresponding CRISPR-based gene silencing agent, a conventional therapy, etc. In some instances, useful antagonists may be identified by screening candidate compounds, including but not limited to e.g., where libraries of compounds (e.g., small molecule chemical libraries) are screened in a high throughput manner.

In some instances, the agent is a granulin (GRN) agonist. Useful GRN agonists include but are not limited to e.g., GRN protein or fragments thereof (e.g., GRN peptide fragments), GRN encoding nucleic acids (e.g., DNA, RNA, modified DNA/RNA containing non-naturally occurring DNA/RNA analogs, and the like), small molecule GRN agonists, or the like.

The GRN gene (also known as Granulins, progranulin (PGRN), Proepithelin (PEPI), Acrogranin, and Glycoprotein of 88 Kda (GP88)) encodes for granulin proteins by way of a progranulin polypeptide. Each granulin protein is cleaved from the precursor progranulin, which in humans is 593 amino acids long. Granulins have cytokine-like activity and are considered to play a role in inflammation, would repair, and tissue remodeling. Granulins are also thought to be involved in development, cell proliferation, and protein homeostasis. Progranulin expression is typically low in early development, but expression increases as cells mature. Isoform 1 of Human GRN (NP_002078.1; Uniprot P28799) has the following amino acid sequence, encoded by NM_002087.3:

(SEQ ID NO: 1)
MWTLVSWVALTAGLVAGTRCPDGQFCPVACCLDPGGASYSCCRPLLDKWP
TTLSRHLGGPCQVDAHCSAGHSCIFTVSGTSSCCPFPEAVACGDGHHCCP
RGFHCSADGRSCFQRSGNNSVGAIQCPDSQFECPDFSTCCVMVDGSWGCC
PMPQASCCEDRVHCCPHGAFCDLVHTRCITPTGTHPLAKKLPAQRTNRAV
ALSSSVMCPDARSRCPDGSTCCELPSGKYGCCPMPNATCCSDHLHCCPQD
TVCDLIQSKCLSKENATTDLLTKLPAHTVGDVKCDMEVSCPDGYTCCRLQ
SGAWGCCPFTQAVCCEDHIHCCPAGFTCDTQKGTCEQGPHQVPWMEKAPA
HLSLPDPQALKRDVPCDNVSSCPSSDTCCQLTSGEWGCCPIPEAVCCSDH
QHCCPQGYTCVAEGQCQRGSEIVAGLEKMPARRASLSHPRDIGCDQHTSC
PVGQTCCPSLGGSWACCQLPHAVCCEDRQHCCPAGYTCNVKARSCEKEVV
SAQPATFLARSPHVGVKDVECGEGHFCHDNQTCCRDNRQGWACCPYRQGV
CCADRRHCCPAGFRCAARGTKCLRREAPRWDAPLRDPALRQLL.

Human GRN isoform 2 is an alternative form relative to isoform 1, differing from the above amino acid sequence in that amino acids 377-531 are absent. Human GRN isoform 3 is an alternative form relative to isoform 1, differing from the above amino acid sequence in that amino acids 1-71 are replaced with MAITAAHGASTAVQTGDPASKDQVTTPWVPSSALIVSSNARTSPRAVLWSMAPGGAAPCPR LPAVKTGCTA (SEQ ID NO:2) and amino acids 72-251 are absent.

Natural variants (i.e., natural mutants) of GRN include, but may not be necessarily limited to, a A to D substitution at position 9; a R to W substitution at position 19; a R to W substitution at position 55; a A to T substitution at position 69; a deletion at position 119; a S to Y substitution at position 120; a T to M substitution at position 182; a C to s substitution at position 221; a P to L substitution at position 275; a D to N substitution at position 376; a S to L substitution at position 398; a R to Q substitution at position 433; a G to A substitution at position 515; and a R to H substitution at position 564. Related proteins include but are not limited to rodent homologs such as but not limited to e.g., M. musculus Grn gene product (NP_032201.2) and the like.

In some instances, the agent is a solute carrier family 33 member 1 (SLC33A1) agonist. Useful SLC33A1 agonists include but are not limited to e.g., SLC33A1 protein or fragments thereof (e.g., SLC33A1 peptide fragments), SLC33A1 encoding nucleic acids (e.g., DNA, RNA, modified DNA/RNA containing non-naturally occurring DNA/RNA analogs, and the like), small molecule SLC33A1 agonists, or the like.

The SLC33A1 gene (also known as AT1, AT-1, ACATN, SPG42, and CCHLND) encodes the protein acetyl-coenzyme A (acetyl-CoA) transporter 1, which is also known as solute carrier family 33 member 1 (SLC33A1). SLC33A1 is a 6 to 10 transmembrane domain protein with and a leucine zipper motif in transmembrane domain III that is necessary for the 0-acetylation of gangliosides. Isoform 1 of Human SLC33A1 (NP_001177921.1; Uniprot 000400) has the following amino acid sequence, encoded by NM_001190992.1:

(SEQ ID NO: 3)
MSPTISHKDSSRQRRPGNFSHSLDMKSGPLPPGGWDDSHLDSAGREGDRE
ALLGDTGTGDFLKAPQSFRAELSSILLLLFLYVLQGIPLGLAGSIPLILQ
SKNVSYTDQAFFSFVFWPFSLKLLWAPLVDAVYVKNFGRRKSWLVPTQYI
LGLFMIYLSTQVDRLLGNTDDRTPDVIALTVAFFLFEFLAATQDIAVDGW
ALTMLSRENVGYASTCNSVGQTAGYFLGNVLFLALESADFCNKYLRFQPQ
PRGIVTLSDFLFFWGTVFLITTTLVALLKKENEVSVVKEETQGITDTYKL
LFAIIKMPAVLTFCLLILTAKIGFSAADAVTGLKLVEEGVPKEHLALLAV
PMVPLQIILPLIISKYTAGPQPLNTFYKAMPYRLLLGLEYALLVWWTPKV
EHQGGFPIYYYIVVLLSYALHQVTVYSMYVSIMAFNAKVSDPLIGGTYMT
LLNTVSNLGGNWPSTVALWLVDPLTVKECVGASNQNCRTPDAVELCKKLG
GSCVTALDGYYVESIICVFIGFGWWFFLGPKFKKLQDEGSSSWKCKRNN.

Natural variants (i.e., natural mutants) of SLC33A1 include, but may not be necessarily limited to, an A to P substitution at position 110; a S to R substitution at position 113; a D to G substitution at position 171; and a V to A substitution at position 400. Related proteins include but are not limited to rodent homologs such as but not limited to e.g., M. musculus acetyl-coenzyme A transporter 1 (NP_056543.2).

In some instances, the agent is a vacuolar protein sorting-associated protein 35 (VPS35) agonist. Useful VPS35 agonists include but are not limited to e.g., VPS35 protein or fragments thereof (e.g., VPS35 peptide fragments), VPS35 encoding nucleic acids (e.g., DNA, RNA, modified DNA/RNA containing non-naturally occurring DNA/RNA analogs, and the like), small molecule VPS35 agonists, or the like.

The VPS35 gene (also known as hVPS35, Maternal-embryonic 3, and Vesicle protein sorting 35) encodes the protein vacuolar protein sorting-associated protein 35. VPS35 is part of a larger group of vacuolar protein sorting (VPS) genes. The VPS45 protein is a part of a large multimeric complex, known as the retromer complex, which is involved in retrograde transport of proteins from endosomes to the trans-Golgi network. The retromer complex has been shown to mediate retrieval of various transmembrane receptors, e.g. the cation-independent mannose 6-phosphate receptor. VPS35 is the large subunit of the retromer complex. Isoform 1 of Human VPS35 (NP_060676.2; Uniprot Q96QK1) has the following amino acid sequence, encoded by NM_018206.5:

(SEQ ID NO: 4)
MPTTQQSPQDEQEKLLDEAIQAVKVQSFQMKRCLDKNKLMDALKHASNML
GELRTSMLSPKSYYELYMAISDELHYLEVYLTDEFAKGRKVADLYELVQY
AGNIIPRLYLLITVGVVYVKSFPQSRKDILKDLVEMCRGVQHPLRGLFLR
NYLLQCTRNILPDEGEPTDEETTGDISDSMDFVLLNFAEMNKLWVRMQHQ
GHSRDREKRERERQELRILVGTNLVRLSQLEGVNVERYKQIVLTGILEQV
VNCRDALAQEYLMECIIQVFPDEFHLQTLNPFLRACAELHQNVNVKNIII
ALIDRLALFAHREDGPGIPADIKLFDIFSQQVATVIQSRQDMPSEDVVSL
QVSLINLAMKCYPDRVDYVDKVLETTVEIFNKLNLEHIATSSAVSKELTR
LLKIPVDTYNNILTVLKLKHFHPLFEYFDYESRKSMSCYVLSNVLDYNTE
IVSQDQVDSIMNLVSTLIQDQPDQPVEDPDPEDFADEQSLVGRFIHLLRS
EDPDQQYLILNTARKHFGAGGNQRIRFTLPPLVFAAYQLAFRYKENSKVD
DKWEKKCQKIFSFAHQTISALIKAELAELPLRLFLQGALAAGEIGFENHE
TVAYEFMSQAFSLYEDEISDSKAQLAAITLIIGTFERMKCFSEENHEPLR
TQCALAASKLLKKPDQGRAVSTCAHLFWSGRNTDKNGEELHGGKRVMECL
KKALKIANQCMDPSLQVQLFIEILNRYIYFY.

Natural variants (i.e., natural mutants) of VPS35 include, but may not necessarily be limited to, a G to S substitution at position 51; a M to I substitution at position 57; a T to R substitution at position 82; a I to M substitution at position 241; a P to S substitution at position 316; a Q to P substitution at position 469; a R to W substitution at position 524; a V to D substitution at position 602; a D to N substitution at position 620; a A to V substitution at position 737; and a L to M substitution at position 774. Related proteins include but are not limited to rodent homologs such as but not limited to e.g., M. musculus Vps35 gene product (NP_075373.1) and R. norvegicus maternal embryonic message 3 (NP_001099188.2).

In some instances, the agent is a Sorting nexin-17 (SNX17) antagonist. Useful SNX17 antagonists include but are not limited to e.g., SNX17 antibodies (e.g., SNX17 neutralizing antibodies, SNX17 function blocking antibodies, etc.), SNX17 gene silencing agents (e.g., CRISPR-based gene silencing agents), SNX17 interfering nucleic acids, small molecule SNX17 antagonists, and the like. In some instances, antagonists may be employed which permanently or transiently disrupt expression from an endogenous SNX17 locus, e.g., through genetic modification of the locus.

The SNX17 gene (also known as KIAA0064) encodes the protein Sorting nexin-17. SNX17 binds to NPxY sequences in the cytoplasmic tails of target cargos and is a critical regulator of endosomal recycling of numerous receptors, channels, and other transmembrane proteins. SNX17 plays a role in the sorting of endocytosed LRP1 and APP, prevents their degradation, and regulates cell surface levels of APP and LRP1. SNX17 recycles internalized integrins ITGB1, ITGB5 and their associated alpha subunits, preventing them from lysosomal degradation and Interacts with membranes containing phosphatidylinositol 3-phosphate (PtdIns(3P)). Isoform 1 of Human SNX17 (NP_055563.1; Uniprot Q15036) has the following amino acid sequence, encoded by NM_014748.3:

(SEQ ID NO: 5)
MHFSIPETESRSGDSGGSAYVAYNIHVNGVLHCRVRYSQLLGLHEQLRKE
YGANVLPAFPPKKLFSLTPAEVEQRREQLEKYMQAVRQDPLLGSSETFNS
FLRRAQQETQQVPTEEVSLEVLLSNGQKVLVNVLTSDQTEDVLEAVAAKL
DLPDDLIGYFSLFLVREKEDGAFSFVRKLQEFELPYVSVTSLRSQEYKIV
LRKSYWDSAYDDDVMENRVGLNLLYAQTVSDIERGWILVTKEQHRQLKSL
QEKVSKKEFLRLAQTLRHYGYLRFDACVADFPEKDCPVVVSAGNSELSLQ
LRLPGQQLREGSFRVTRMRCWRVTSSVPLPSGSTSSPGRGRGEVRLELAF
EYLMSKDRLQWVTITSPQAIMMSICLQSMVDELMVKKSGGSIRKMLRRRV
GGTLRRSDSQQAVKSPPLLESPDATRESMVKLSSKLSAVSLRGIGSPSTD
ASASDVHGNFAFEGIGDEDL.

Isoform 3 of Human SNX17 (NP_001253989.1) has the following amino acid sequence, encoded by NM_001267060.1:

(SEQ ID NO: 6)
MHFSIPETESRSGDSGGSAYVLRKEYGANVLPAFPPKKLFSLTPAEVEQR
REQLEKYMQAVRQDPLLGSSETFNSFLRRAQQETQQVPTEEVSLEVLLSN
GQKVLVNVLTSDQTEDVLEAVAAKLDLPDDLIGYFSLFLVREKEDGAFSF
VRKLQEFELPYVSVTSLRSQEYKIVLRKSYWDSAYDDDVMENRVGLNLLY
AQTVSDIERGWILVTKEQHRQLKSLQEKVSKKEFLRLAQTLRHYGYLRFD
ACVADFPEKDCPVVVSAGNSELSLQLRLPGQQLREGSFRVIRMRCWRVTS
SVPLPSGSTSSPGRGRGEVRLELAFEYLMSKDRLQWVTITSPQAIMMSIC
LQSMVDELMVKKSGGSIRKMLRRRVGGTLRRSDSQQAVKSPPLLESPDAT
RESMVKLSSKLSAVSLRGIGSPSTDASASDVHGNFAFEGIGDEDL.

Related proteins include but are not limited to rodent homologs such as but not limited to e.g., M. musculus Snx17 gene product (NP_710147.1) and the like.

Various agents may be employed in treating the herein described neurodegenerative disorders, the progression of which are based, at least in part, in the accumulation of lipid droplets in LAM. For example, in some instances, Parkinson's disease may be treated by administering to a subject an effective amount of a SLC33A1 agonist and/or a SNX17 antagonist. In some instances, frontotemporal dementia may be treated by administering to a subject an effective amount of a SLC33A1 agonist and/or a SNX17 antagonist. In some instances, frontotemporal dementia may be treated by administering to a subject an effective amount of a SLC33A1 agonist, a VPS35 agonist and/or a SNX17 antagonist. In some instances, Alzheimer's disease may be treated by administering to a subject an effective amount of a VPS35 agonist. In some instances, an agent described herein may be employed for treating a neurodegenerative disorder that is a tauopathy, e.g., a VPS35 agonist may be employed for treating a tauopathy. In some instances, an agent described herein may be employed for treating a neurodegenerative disorder that is not a tauopathy, e.g., a SLC33A1 agonist and/or a SNX17 antagonist may be employed for treating a neurodegenerative disorder that is not a tauopathy.

The compositions (e.g., those including one or more agents that reduce the prevalence of LAM and/or reduce lipid droplet accumulation in LAM) of this disclosure can be supplied in the form of a pharmaceutical composition. Any suitable pharmaceutical composition may be employed, described in more detail below. As such, in some instances, methods of the present disclosure may include administering one or more agents in a composition comprising an excipient (e.g., an isotonic excipient) prepared under sufficiently sterile conditions for administration to a mammal, e.g., a human.

Subjects treated according to the herein described methods may or may not include one or more mutations in a gene to which the administered agent is targeted, including genes targeted by either agonists or antagonists. For example, in some instances, a subject may be “wildtype” (i.e., not mutated) at the genetic locus (i.e., the gene) associated with the administered agent (e.g., wildtype at the SNX17 locus when administered a SNX17 antagonist). Correspondingly, in some instances, a subject may be “mutant” (i.e., mutated) at the genetic locus (i.e., the gene) associated with the administered agent (e.g., mutant at the VPS35 locus when administered a VPS35 agonist). By “mutant”, as specifically used in this context, is meant that the locus is sufficiently altered such that the encoded protein is sufficiently altered to substantially affect the function of the protein. In some instances, a mutation that may be present or absent in a subject may be, but are not necessarily limited to, one or more of the mutations described herein.

As used herein, the term “wildtype locus” generally refers to a gene, or sub-portion thereof, in the subject that is not mutated, or not substantially mutated (e.g., at either allele) so as to affect the function of the gene. Accordingly, a wildtype locus may contain the common (i.e., most prevalent, normal, etc.) sequence of the gene, or essentially the common sequence of the gene, without mutation, or without substantial mutation, affecting the function of the gene. The “common sequence”, as used in this context, will generally refer to the gene sequence as it most frequently occurs in a natural population. In some instances, common sequences may be represented by a reference sequence, e.g., a reference sequence as it appears in a sequence database, such as but not limited to e.g., GenBank database (NCBI), UniProt database (EBI/SIB/PIR), or the like. In some instances, a wildtype locus may be identical or substantially identical to a reference sequence.

For example, in some instances, a subject treated according to the methods of the present disclosure may be wildtype at one or more loci of target genes identified herein. Correspondingly, without limitation, a subject treated according to the methods of the present disclosure may be wildtype at the GRN locus, wildtype at the SLC33A1 locus, wildtype at the VPS35 locus, wildtype at the SNX17 locus, or wildtype at a combination of loci thereof.

In some instances, a subject treated according to the methods of the present disclosure may be mutant at one or more loci of target genes identified herein. Correspondingly, without limitation, a subject treated according to the methods of the present disclosure may be mutant at the GRN locus, mutant at the SLC33A1 locus, mutant at the VPS35 locus, mutant at the SNX17 locus, or mutant at a combination of loci thereof.

Administration of an agent to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Formulations for these dosage forms are described herein.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to the subject, the amount or dosage is effective when administered for a suitable period of time, such as one week or longer, including two weeks or longer, such as 3 weeks or longer, 4 weeks or longer, 8 weeks or longer, etc., so as to evidence a reduction in the disorder, e.g., a reduction in a symptom of the disorder or in a marker of disease pathology. For example, an effective dose is the dose that, when administered for a suitable period of time, such as at least about one week, and maybe about two weeks, or more, up to a period of about 3 weeks, 4 weeks, 8 weeks, or longer, will reduce a symptom of the disorder e.g., by about 10% or more, by about 20% or more, e.g., by 30% or more, by 40% or more, or by 50% or more, in some instances by 60% or more, by 70% or more, by 80% or more, or by 90% or more, e.g., will halt, progression of the disorder in the subject. In some instances, an effective amount or dose of active agent will not only slow or halt the progression of the disease condition but will also induce the reversal of the condition, i.e., will cause an improvement in the neurological health of the subject. For example, in some instances, an effective amount is the amount that when administered for a suitable period of time, e.g., at least about one week, and/or about two weeks, or more, up to a period of about 3 weeks, 4 weeks, 8 weeks, or longer will improve, stabilize, or at least reduce the progression of a disorder in subject, for example 1.5-fold, 2-fold, 3-fold, 4-fold, 5-fold, in some instances 6-fold, 7-fold, 8-fold, 9-fold, or 10-fold or more relative to the subject's condition prior to administration.

In some instances, in those embodiments where an effective amount of an active agent is administered to the subject, the amount or dosage is effective when administered for a suitable period of time to result in a rejection in the prevalence of LAM in the subject. Such a reduction may manifest in various ways, including but not limited to e.g., a reduction in the number of LAM cells (i.e., a reduction in the size of the LAM population), a reduction in the amount of lipid droplets present (e.g., a reduction in the lipid droplet load present in the LAM population), or the like. In some instances, methods of the present disclosure may results in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., reduction in the prevalence of LAM. In some instances, methods of the present disclosure may results in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., reduction in the lipid droplet load, including e.g., the LAM lipid droplet load. Various methods of assessing the presence of LAM and/or measuring the lipid droplet load may be employed, including invasive and non-invasive techniques, such as but not limited to e.g., lipid imaging in biopsy specimens and/or MR lipid imaging (e.g., as adapted from that described in Delikatny et al., NMR Biomed. (2011) 24(6): 592-611, the disclosure of which is incorporated herein by reference in its entirety).

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent that inhibits a target gene (e.g., a SNX17 target gene, and the like) and/or compositions is an amount that is sufficient, when administered to the individual, to palliate, ameliorate, stabilize, reverse, prevent, slow or delay the progression of the disease state (e.g., neurodegenerative disease, etc.) by, for example, inhibiting the formation of lipid droplets, inhibiting the genesis of lipid droplet accumulating microglia (LAM), inhibiting the growth/proliferation of LAM, inducing death of LAM, or otherwise preventing the clinical progressing of a neurodegenerative disorder present in the subject.

In some instances, methods of the present disclosure may include administering to the subject a general inhibitor of lipid droplet formation, such as but not limited to e.g., a lipogenesis inhibitor. For example, in some instances, a subject may be administered an agent that reduces the prevalence of LAM (e.g., an agonist or antagonist of one of the target described herein) and/or lipid droplet accumulation of LAM, and further a general inhibitor of lipid droplet formation, such as but not limited to e.g., a small molecule inhibitor of lipogenesis. Any convenient general inhibitor of lipid droplet formation may be employed, including but not limited to e.g., modulators of fatty acid biosynthesis.

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a subject compound or pharmaceutical composition can be determined by one of skill in the art, with a goal of achieving local (e.g., tissue) concentrations that are at least as high as the 1050 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, Md. 20857; (available at www(dot)fda(dot)gov/cder/guidance/index(dot)htm, the disclosure of which is incorporated herein by reference).

Conversion of Animal Doses to Human Equivalent Doses Based on Body Surface Area

| | | To Convert Animal Dose in mg/kg to | |
|---|---|---|---|
| | To Convert Animal Dose in | HED^{a} in mg/kg, Either: | |
| | mg/kg to Dose in mg/m², | Divide | Multiply |
| Species | Multiply by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula:
HED = animal dose in mg/kg × (animal weight in kg/human weight in kg)0.33.
^{b}This km value is provided for reference only since healthy children will rarely be volunteers for phase 1 trials.
^{c}For example, cynomolgus, rhesus, and stumptail.

Pharmaceutical Compositions

A pharmaceutical composition comprising a subject compound (i.e., an agonist or an antagonist) may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

A subject compound may be administered to the host using any convenient means capable of resulting in the desired reduction in disease condition or symptom. Thus, a subject compound can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the infection to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a subject compound can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

A subject compound can be utilized in aerosol formulation to be administered via inhalation. A subject compound can be formulated into pressurized acceptable propellants such as dichlorodifluoromethane, propane, nitrogen and the like.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are solidified at room temperature.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

In some instances, methods of treating a subject as described herein may include administering to the subject an effective amount of an agent that reduces lipid droplet formation, reduces the prevalence of LAM, and/or reduces lipid droplet accumulation in LAM, as identified in a method of screening described herein.

Methods of Screening

As summarized above, the methods of the present disclosure also include methods of identifying lipid droplet-associated target genes, including target genes that are positive and negative regulators of lipid droplet formation. Useful screens for identifying positive and negative regulators of lipid droplet formation may include, but are not limited to e.g., genetic screens, such as but not limited to e.g., genes employing silencing reagents, such as but not limited to e.g., CRISPR-based gene silencing reagents.

By “lipid droplet-associated target gene”, as used herein, is meant a gene that is involved, either positively or negatively, in the formation or lipid droplets, the formation of LAM, and/or the accumulation of lipid droplets in LAM. Lipid droplet-associated target genes may be identified from various populations of target genes and in some instances, a lipid droplet-associated target gene may be identified from a population of target genes identified in a genetic screen selecting for or against lipid droplet formation, such as but not limited to e.g., a CRISPR-based gene silencing screen selecting for or against lipid droplet formation.

As summarized above, in some embodiments, screens of the present disclosure may include contacting a population of microglia with a plurality of CRISPR-based gene silencing agents targeting a plurality of target genes. Accordingly, in such an approach a variety of different genes may be silenced and analyzed in a pooled or multiplexed format as desired. Various populations of microglia may be employed in such methods, including where such microglia are primary cells or cultured cells. In some instances, primary cells obtained from a subject may be employed. In some instances, cultured cells of a microglial cell culture line may be employed. An employed population of microglia may, regardless of whether the cells are primary or cell-line derived, include LAM cells in the population.

By “CRISPR-based gene silencing agent” is meant one or more agents that when delivered to a cell cause the directed silencing of a target gene by CRISPER/Cas9-based nuclease activity. Accordingly, in some instances, a CRISPR-based gene silencing agent may include a guide RNA (gRNA) having sequence that specifically targets a Cas9 nuclease to a specific target gene. CRISPR/Cas9-based silencing of a target gene may include delivery of a Cas9 polypeptide or a Cas9 polypeptide encoding nucleic acid to the subject cells. For example, in some instances, a vector that includes a nucleic acid that encodes a Cas9 nuclease may be delivered to the subject cells before, during or after the cell is contacted with a CRISPR-based gene silencing agent such that the encoded Cas9 nuclease is expressed when the CRISPR-based gene silencing agent is present within the cell. In some instances, the cell may be genetically modified with a nucleic acid encoding a Cas9 nuclease such that the encoded Cas9 nuclease is expressed (e.g., conditionally expressed, constitutively expressed, etc.) when the CRISPR-based gene silencing agent is present within the cell. Accordingly, CRISPR/Cas9-based silencing of the present methods may employ a Cas9 nuclease that is stably or transiently expressed including e.g., where a nucleic acid encoding the Cas9 nuclease is transiently or stably present within the cell line. In some instances, Cas9 polypeptide may be delivered to the subject cells, i.e., without the need to express the Cas9 polypeptide within the cells. CRISPR-based gene silencing agents will vary and may include e.g., vector (e.g., virus (e.g., lentivirus), plasmid, etc.) containing and/or expressing one or more gRNAs. Methods of delivery of CRISPR-based gene silencing agents will similarly vary any may include e.g., transfection, electroporation, lipofection, etc.

CRISPR-based gene silencing agents of the present disclosure may be directed to essentially any element of a subject genome including e.g., protein-coding and non-protein coding elements of the subject genome. In some instances, e.g., where a plurality of CRISPR-based gene silencing agents is employed, the plurality of CRISPR-based gene silencing agents may collectively target all or essentially all genes of the subject genome (i.e., genome-wide targeting). In some instances, targeted non-protein coding elements may include but are not limited to e.g., promoters, enhancers, non-coding RNAs, and the like. In some instances, the targets of one or more CRISPR-based gene silencing agents may include proteins involved lipogenesis, fatty acid metabolism, lipid trafficking, or the like. In some instances, the function of the targeted gene may be unknown. In some instances, the targets of one or more CRISPR-based gene silencing agents may include proteins involved in processes other than lipogenesis, fatty acid metabolism, lipid trafficking, including but not limited to e.g., protein processing/trafficking, neuronal cell differentiation, cellular homeostasis, cellular metabolism, and the like.

The subject methods of identifying a lipid droplet-associated target gene may be performed in multiplex fashion, including e.g., where contacting one or more populations of microglia with a CRISPR-based gene silencing agent targeting a target gene may include contacting the populations with a plurality of CRISPR-based gene silencing agents targeting a plurality of different target genes. Such pluralities of target genes will vary and may include e.g., where the plurality includes all or essentially all of the genes of a genome of a subject such as a mammal (e.g., a mouse, rat, primate, human, etc.). In some instances, a plurality of target genes may include only the genes of a particular functional group including e.g., genes involved in lipogenesis, fatty acid metabolism, lipid trafficking, or the like. In some instances, a plurality of CRISPR-based gene silencing agents may be referred to as a library of CRISPR-based gene silencing agents and contacting a population of cells, as described herein, may include contacting the cells with the library.

In some instances, a method of screening may include inducing lipid droplet formation, including where lipid droplet formation is induced in a population of microglia. Lipid droplet formation may be induced by a variety of means. In some instances, lipid droplet formation may be induced using a lipopolysaccharide (LPS), including but not limited to e.g., by contacting a population of cells, e.g., microglia, with LPS.

In some instances, a method of screening may include cell selection, including positive and/or negative selection. For example, in some instances lipid droplet-deficient cells may be selected and further analyzed, e.g., by sequencing or other methods. Cells may be selected based on an ability or inability to form lipid droplets, with or without induction of lipid droplet formation. For example, in some instances, cells may be selected that form lipid droplets, e.g., by staining cells for lipids to identify lipid droplets and sorting (e.g., FACS sorting) cells that have enhanced levels of lipid droplets. In some instances, cells may be selected that do not form droplets, e.g., by staining cells for lipids to identify lipid droplets and sorting (e.g., FACS sorting) cells that have reduced levels of lipid droplets.

In some instances, cells that do not form lipid droplets or have reduced levels of lipid droplets may be selected for by removing or ablating cells that do form lipid droplets and/or cells that form enhanced levels of lipid droplets. For example, in some instances, cells that form lipid droplets may be ablated (e.g., chemically ablated, laser ablated, photo-ablated, or the like) and the remaining cells may be collected. Correspondingly, the collected cells may be enriched for cells that do not form lipid droplets of form reduced levels of lipid droplets. In some instances, a method of screening of the present disclosure may make use of lipid-based photoablation, including but not limited to e.g., a method that employs a photosensitizer, such as but not limited to e.g., iodo-BODIPY (iBP).

In some instances, a method of screening may include sequencing, including but not limited to e.g., sequencing cells selected in the screen. Any convenient and appropriate method of sequencing may be employed including but not limited to e.g., RNA-seq.

In some instances, sequencing may provide for identifying a lipid droplet-associated target gene. For example, in some instances, the sequencing results may be quantified, including e.g., where the read number, which may be representative of the relative amount of nucleic acid transcript associated with a particular target gene, is measured and used to determine the relative amount that a particular target gene transcript is represented in a sample. A low read number (or the absence of reads) associated with a particular target gene may indicate that the target gene transcript is under represented or not present in the sample (e.g., due to genetic ablation of the target gene, e.g., by a gene silencing reagent, or ablation of cells expressing the target gene). A high read number (or the presence of reads) associated with a particular target gene may indicate that the target gene is over represented or is present in the sample (e.g., the target gene was not genetically ablated, e.g., by a gene silencing reagent, or cells expressing the target gene are present).

In some embodiments, a subject screen may involve genetic silencing of various target genes in a population of cells, induction of lipid droplet formation, and selection for cells that do not form (or form less) lipid droplets. Accordingly, in such a screen, lipid droplet-forming cells may be under represented or absent and lipid droplet-negative cells (i.e., cells without lipid droplets or having a low amount of lipid-droplets) may be over represented or essentially the only cells present. Correspondingly, sequencing of the remaining or selected lipid droplet-negative cells will reveal over represented and under represented reads for the various target gene transcripts. Where a cell having a silenced target gene is present or over represented in the population of selected lipid droplet-negative cells, the silenced target gene may be identified as a positive regulator of lipid droplet formation. Where a cell having a silenced target gene is absent or under represented in the population of selected lipid droplet-negative cells, the silenced target gene may be identified as a negative regulator of lipid droplet formation.

As summarized above, identified lipid droplet-associated target genes, including target genes that are positive and negative regulators of lipid droplet formation, may be used in the treatment of subjects in need thereof in further methods included in the instant disclosure. Thus, the methods of the present disclosure include methods of treating a neurodegenerative disorder in a subject by administering to the subject an antagonist of a positive regulator of lipid droplet formation, including a positive regulator identified in a screen as described above. Accordingly, the methods of the present disclosure also include methods of treating a neurodegenerative disorder in a subject by administering to the subject an agonist of a negative regulator of lipid droplet formation, including a negative regulator identified in a screen as described above.

Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of treating a neurodegenerative condition in a subject, including by administering to the subject an effective amount of an agent that reduces the prevalence of lipid droplet accumulating microglia. The subject kits may include any combination of components (e.g., reagents, cell lines, etc.) for performing the subject methods, such as e.g., methods of treating a neurodegenerative condition and/or methods of identifying a lipid droplet-associated target gene.

In some embodiments, a subject kit may be employed in a method of identifying a lipid droplet-associated target gene. Such kits may vary and may, but need not necessarily, include one or more microglia cell populations that include LAM. In some instances, a subject kit may include one or more, including a plurality of or a library of, CRISPR-based gene silencing agents. In some embodiments, the subject kits may include a nucleic acid for expressing a Cas9 polypeptide within a microglia cell, such as a microglia cell line or primary microglia. In some instances, a cell line contained within a subject kit may be configured (e.g., genetically modified) to express a Cas9 polypeptide.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

Notwithstanding the appended claims, the disclosure is also defined by the following embodiments:

1. A method of treating a neurodegenerative disorder in a subject in need thereof, the method comprising administering to the subject an effective amount of an agent that reduces the prevalence of lipid droplet accumulating microglia, thereby treating the neurodegenerative disorder.
2. The method according to embodiment 1, wherein the subject is a mammal.
3. The method according to embodiment 2, wherein the neurodegenerative disorder is selected from the group consisting of: Alzheimer's disease, Parkinson's disease, Amyotrophic Lateral Sclerosis, Huntington's disease, and frontotemporal dementia.
4. The method according to any one of embodiments 1 to 3, wherein the agent is a granulin (GRN) agonist, a solute carrier family 33 member 1 (SLC33A1) agonist, a vacuolar protein sorting-associated protein 35 (VPS35) agonist, or a Sorting nexin-17 (SNX17) antagonist.
5. The method according to embodiment 4, wherein the agent is a SLC33A1 agonist and the neurodegenerative disorder is Parkinson's disease or frontotemporal dementia.
6. The method according to embodiment 5, wherein the SLC33A1 agonist is a SLC33A1 peptide, a SLC33A1 encoding nucleic acid, or a small molecule SLC33A1 agonist.
7. The method according to embodiment 4, wherein the agent is a VPS35 agonist and the neurodegenerative disorder is Alzheimer's disease or frontotemporal dementia.
8. The method according to embodiment 7, wherein the VPS35 agonist is a VPS35 peptide, a VPS35 encoding nucleic acid, or a small molecule VPS35 agonist.
9. The method according to embodiment 4, wherein the agent is a SNX17 antagonist and the neurodegenerative disorder is Parkinson's disease or frontotemporal dementia.
10. The method according to embodiment 9, wherein the SNX17 antagonist is a SNX17 antibody, a SNX17 CRISPR-based gene silencing agent, a SNX17 interfering nucleic acid, or a small molecule SNX17 antagonist.
11. The method according to any one of embodiments 1 to 10, wherein the subject comprises a wildtype GRN locus, a wildtype SLC33A1 locus, a wildtype VPS35 locus, a wildtype SNX17 locus, or a combination thereof.
12. The method according to any one of embodiments 1 to 11, wherein the method results in at least a 5% reduction in the prevalence of lipid droplet accumulating microglia.
13. The method according to any one of embodiments to 1 to 12, wherein the method further comprises administering to the subject a general inhibitor of lipid droplet formation.
14. The method according to embodiment 13, wherein the general inhibitor of lipid droplet formation comprises a lipogenesis inhibitor.
15. A method of identifying a lipid droplet-associated target gene, the method comprising:

contacting a population of microglia with a plurality of CRISPR-based gene silencing agents targeting a plurality of target genes;

inducing lipid droplet formation in the contacted population of microglia;

selecting lipid droplet-deficient cells from the induced population and sequencing the selected cells; and

identifying a lipid droplet-associated target gene based on the sequencing, wherein enhanced representation of the target gene indicates a positive regulator of lipid droplet formation and reduced representation of the target gene indicates a negative regulator of lipid droplet formation.

16. The method according to embodiment 15, wherein the population of microglia comprise primary cells.
17. The method according to embodiment 15, wherein the population of microglia comprise cells of a cultured cell line.
18. The method according to any one of embodiments 15 to 17, wherein the population of microglia comprises lipid droplet accumulating microglia.
19. The method according to any one of embodiments 15 to 18, wherein the population of microglia expresses Cas9.
20. The method according to any one of embodiments 15 to 19, wherein inducing lipid droplet formation comprises contacting the population of microglia with lipopolysaccharide.
21. The method according to any one of embodiments 15 to 20, wherein the selecting comprises lipid-based photoablation.
22. A method of treating a neurodegenerative disorder in a subject in need thereof, the method comprising administering to the subject an effective amount of an antagonist of the positive regulator of lipid droplet formation identified according to any one of embodiments 15 to 21.
23. A method of treating a neurodegenerative disorder in a subject in need thereof, the method comprising administering to the subject an effective amount of an agonist of the negative regulator of lipid droplet formation identified according to any one of embodiments 15 to 21.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Example 1: Determining Lipid Droplet Content of Microglia Cells

Recent genetic studies have linked microglia to the pathogenesis of several neurodegenerative diseases. Also, microglia become progressively activated and seemingly dysfunctional with age. In these examples a striking buildup of lipid droplets in microglia with aging in mouse and human brains is demonstrated. These cells are defective in phagocytosis, produce high levels of reactive oxygen species and inflammatory cytokines, and show a transcriptional profile driven by innate inflammation; they are thus distinct from previously reported microglial states. Below, an unbiased CRISPR-Cas9 screen identifies genetic modifiers of lipid droplet formation, including progranulin, SNX17, SLC33A1, and VPS35, genes with genetic linkage to neurodegenerative diseases. Collectively, these examples demonstrate that these lipid droplet accumulating microglia, termed LAM, exert a critical role in age-related neurodegeneration and provide a novel target for therapeutic intervention.

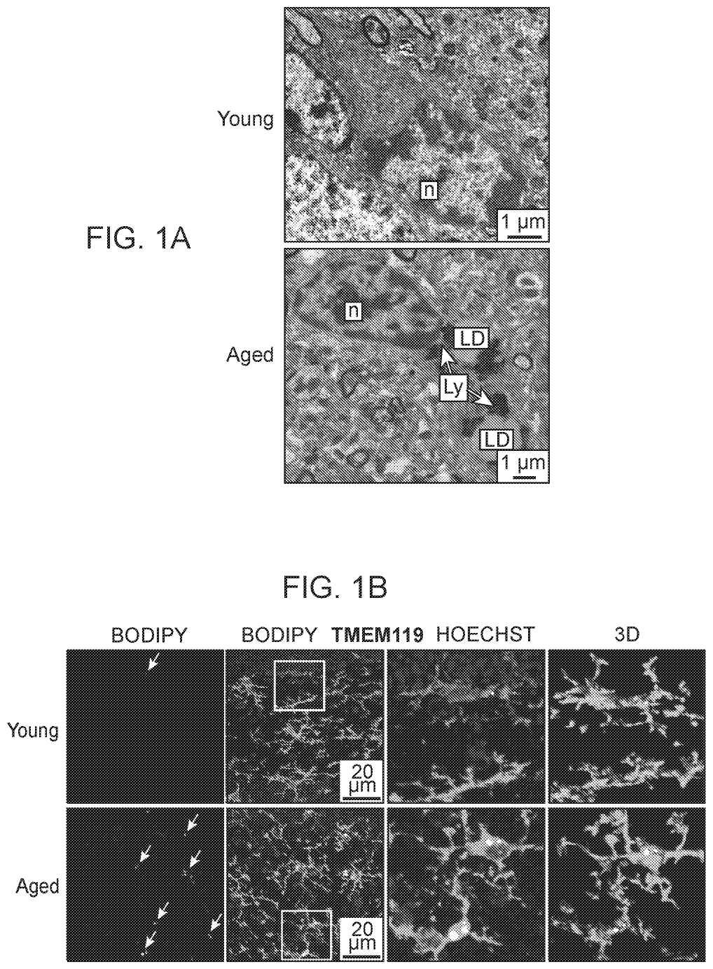

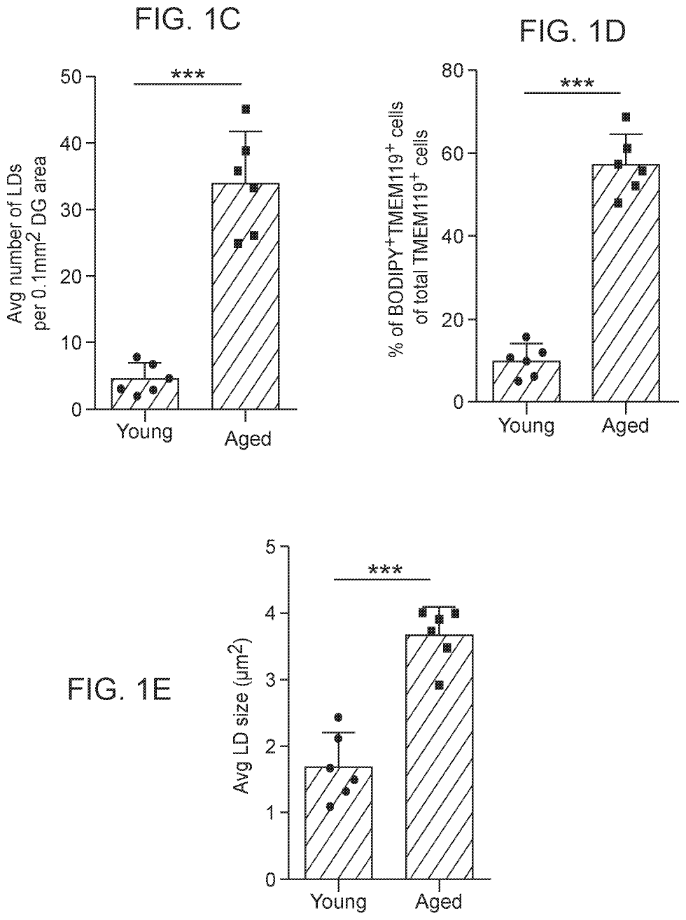

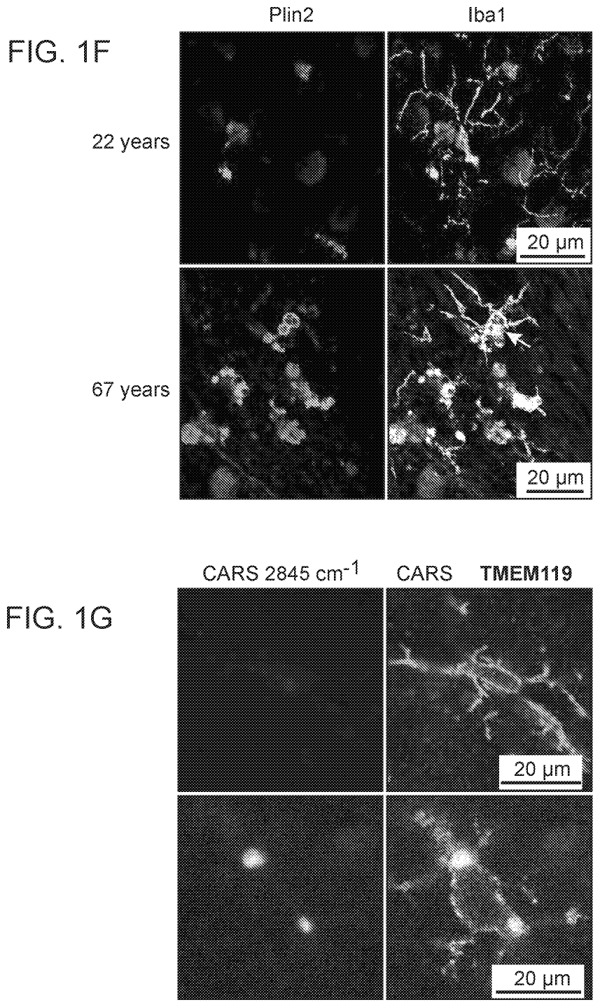

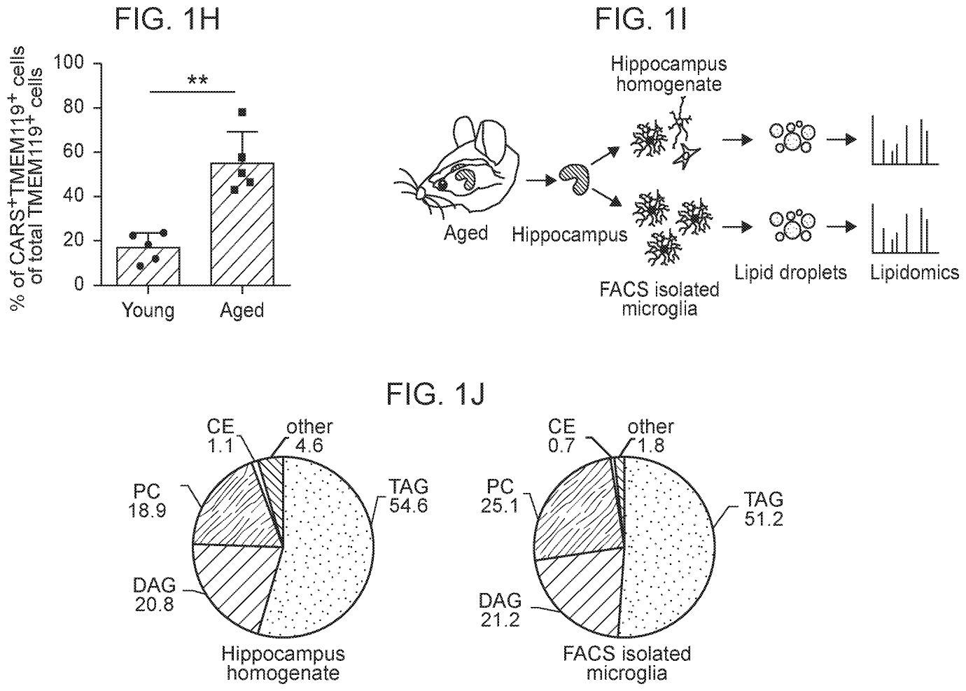

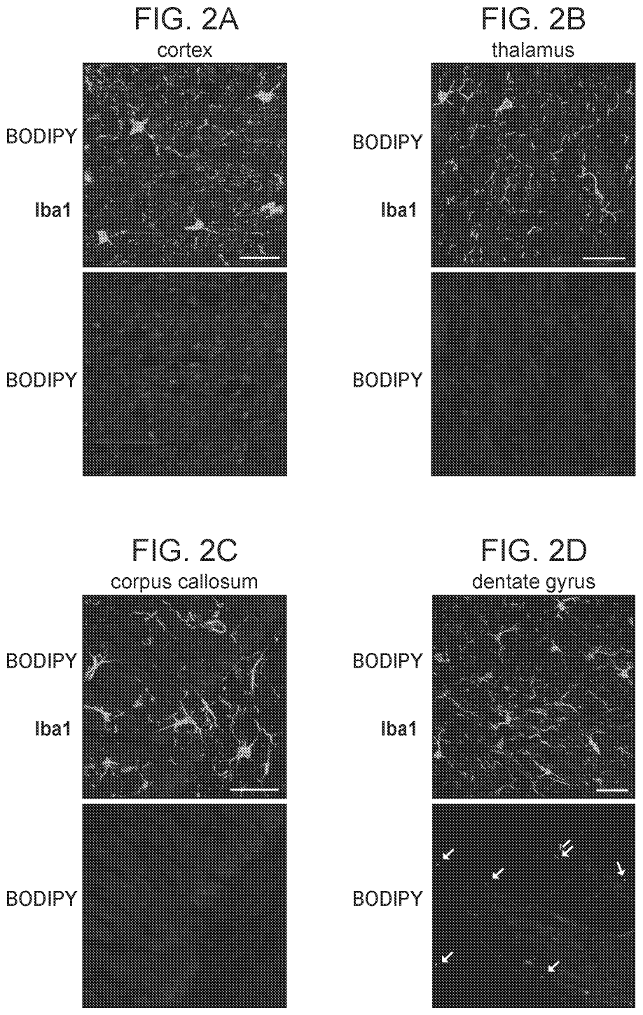

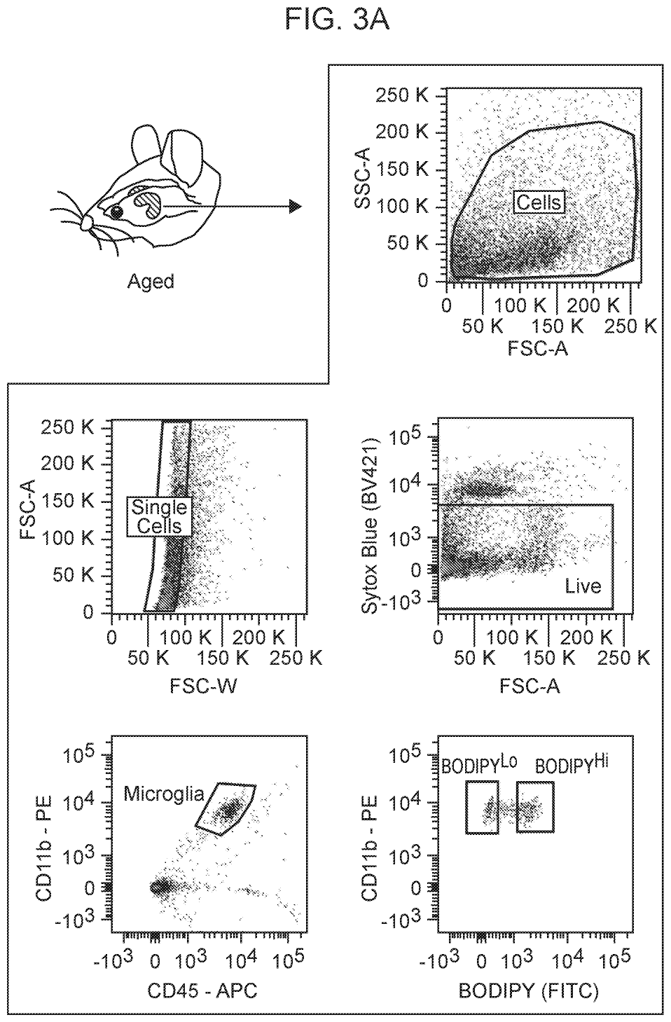

To determine whether microglia contain lipid droplets in the young and aged brain, we analyzed the cytoplasmic content of microglia from 3- and 20-month-old mice by transmission electron microscopy (EM). Lipid droplets (about 1-3 droplets per cell, often closely surrounded by lysosomes) were commonly found in microglia of aged mice, but rarely detected in young mice (FIG. 1, panel A). For detailed quantitative analysis, TMEM119⁺ microglia were co-localized with BODIPY, a dye that specifically labels neutral lipids^{36,37}. It was observed that in the aged brain, BODIPY⁺ microglia were abundant in the hippocampus but scarce in other regions (FIG. 2), and therefore subsequent analyses were focused on the hippocampus. Almost all lipid droplets in the hippocampus co-localized with TMEM119⁺ microglia, indicating that lipid droplets are primarily present in microglia but rare in other cell types. The percentage of BODIPY⁺ TMEM119⁺ microglia in the hippocampus was more than 4-fold higher in aged (51.95%) compared with young (12.08%) microglia, and lipid droplets were significantly bigger in aged than in young microglia (FIG. 1, Panels B-E FIG. 2). Next was investigated whether age-related microglial lipid droplet accumulation might also be relevant for human brain aging. Using an antibody against the lipid droplet surface protein Perilipin2 (Plin2) in human postmortem hippocampus of young adult and aged cognitively normal individuals, it was found that Plin2⁺lba1⁺ microglia were more frequent in aged than in young individuals (FIG. 1, panel F).

To further corroborate the finding of lipid droplet accumulation in aged microglia, coherent anti-Stokes Raman scattering (CARS) microscopy was used, which is a label-free and nonlinear optical technique that enables the identification of types of molecules based on their specific vibrational energy. CARS laser-scanning microscopy at 2,845 cm⁻¹ was performed, which corresponds to the CH₂ stretching frequency for neutral lipids and specifically identifies neutral lipids/lipid droplets³⁸, on TMEM119⁺ immunostained brain sections from young and aged mice. It was found that the numbers of CARS⁺ lipid storing microglia are significantly higher in aged than in young mice (50.76% vs 18.93%) (FIG. 1, Panels G-H).

Lipid droplets are composed of neutral lipids such as triacylglycerols and cholesteryl esters, yet their composition can vary greatly between cell types. Lipid droplets were isolated from FACS-sorted aged hippocampal microglia and whole hippocampus of aged mice to perform lipidomics analysis (FIG. 1, Panel I) and found that lipid droplets of aged microglia are mainly composed of triacylglycerols (TAG, 51.2%) and diacylglycerols (DAG, 21.2%), while cholesteryl esters were almost absent (0.7%) (FIG. 1, Panel J). Indeed, lipid droplets from the whole hippocampus showed a nearly identical lipid distribution, supporting the conclusion that the majority of hippocampal lipid droplets originated from microglia (FIG. 1, Panel B).

### Example 2: Assessing the Transcriptional Phenotype of Lipid Droplet-Containing Microglia Cells

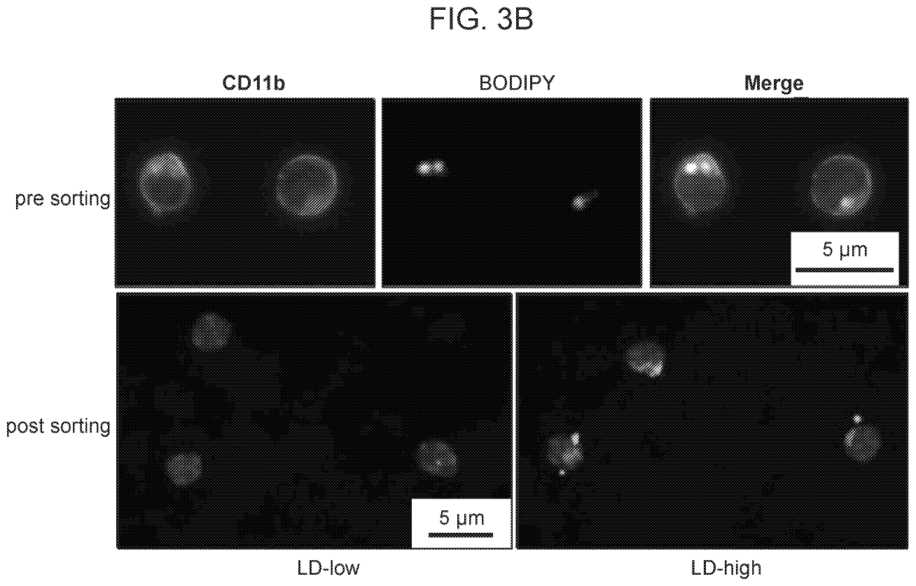

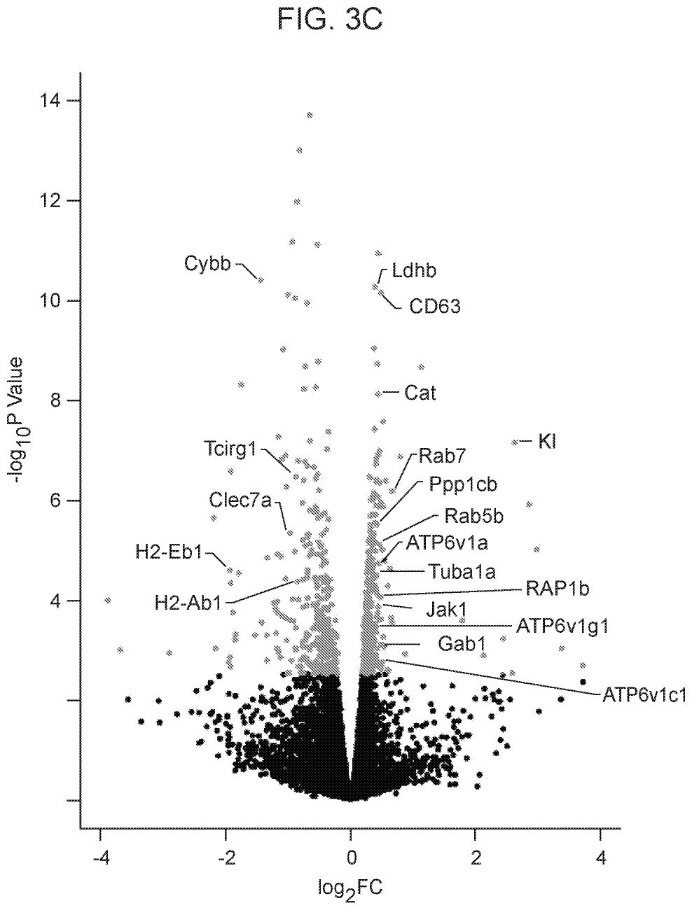

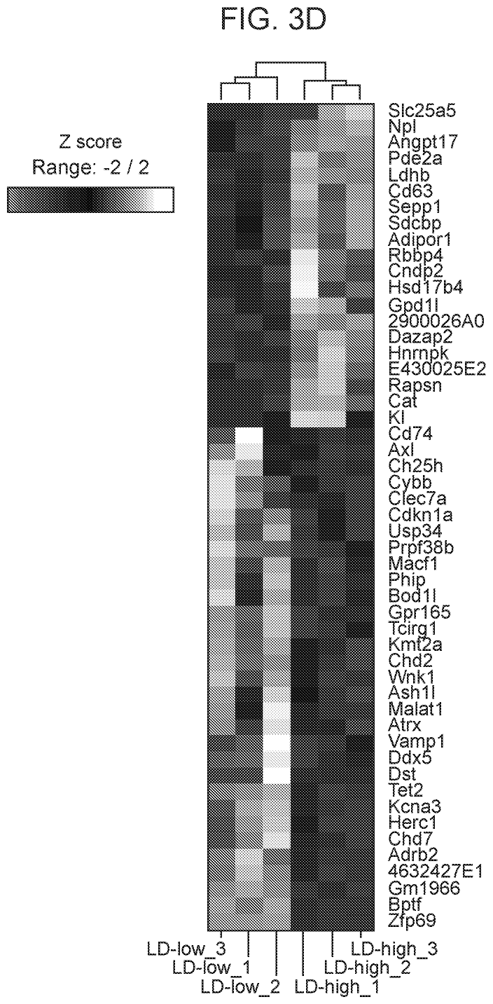

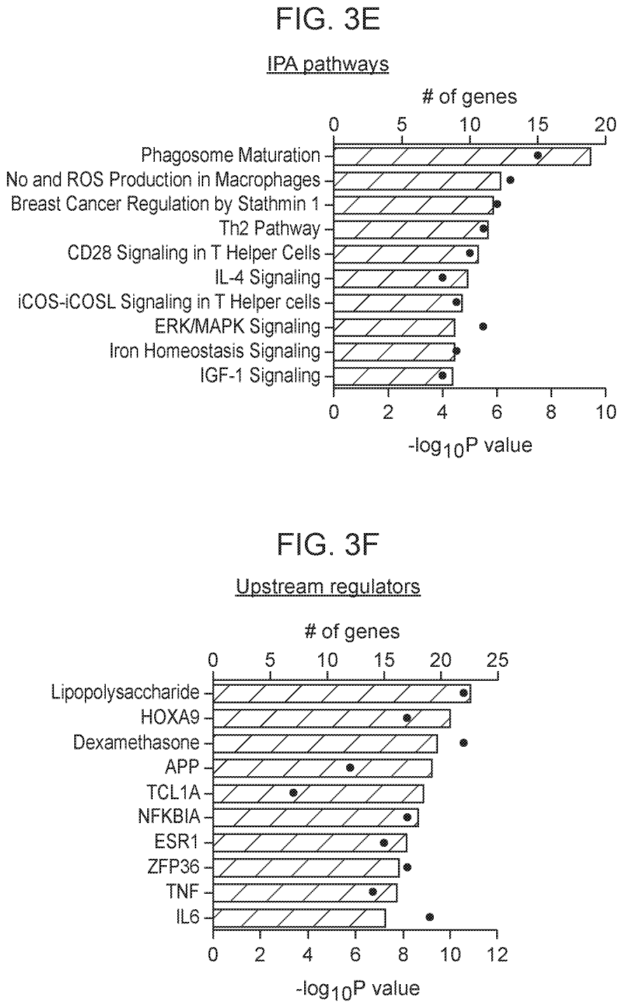

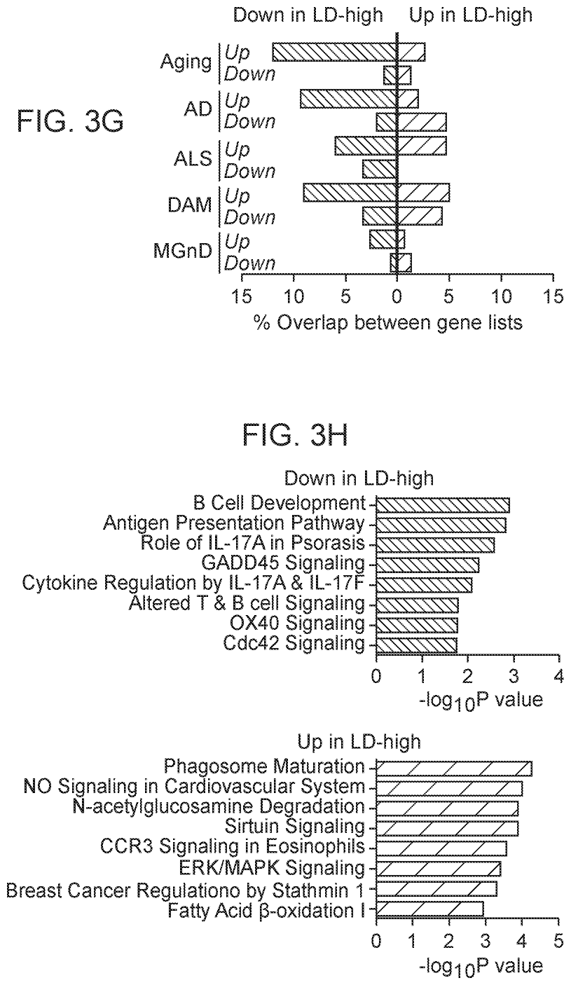

To determine the transcriptional phenotype of lipid droplet-containing microglia in the aged brain, isolated CD11b⁺CD45^{lo} microglia were isolated from the hippocampi of 18-month old mice based on their BODIPY⁺ mean fluorescence intensity and analyzed lipid droplet-low (BODIPY^{lo}; LD-low) and lipid droplet-rich (BODIPY^{hi}; LD-high) microglia by RNA-Sequencing (RNA-Seq) (FIG. 3, Panels A-B). An optimized microglia isolation strategy was used that keeps microglia largely in a non-activated state and therefore prevents unwanted bias towards an activated pro-inflammatory signature³⁹. Unsupervised cluster analysis segregated LD-low from LD-high microglia and revealed prominent differences between their transcriptome with 692 significantly differentially expressed genes (FIG. 3, Panels C-D).

Ingenuity Pathway Analysis (IPA) of differentially expressed genes revealed Phagosome maturation and Production of NO and ROS, two key functions of microglia that become dysregulated with age, as the most significant pathways associated with LD-high microglia (FIG. 3, Panel E). Genes downregulated in LD-high microglia were mainly related to antigen presentation and immune signaling, while pathways associated with genes upregulated in LD-high microglia included phagosome maturation, NO signaling, CCR3 signaling, and fatty acid 8-oxidation (FIG. 3, Panel H). Significantly modulated genes in the phagosome maturation pathway included lysosomal genes (CD63, ATP6V1A, ATP6V1C1, ATP6V1G1, TUBA1), genes involved in vesicular transport (RAB5B, RAB7), and CLEC7A, a gene involved in phagocytic cup formation. In addition, most genes linked to NO and ROS generation (e.g. CAT, KL, PPP1CB, JAK, RAP1B) were upregulated in LD-high microglia (FIG. 3, Panel C). Furthermore, LD-high microglia were enriched in lipid droplet related genes, including the lipid droplet specific gene Perilipin 3 (PLIN3), the lipid transporter VAP, the ATP citrate synthase ACLY, which is involved in lipogenesis, the endosomal/lysosomal gene LAMP1, and several Ras-related small GTPase Rab genes (Supplementary Table 1). Next, the IPA upstream analysis module was used to predict upstream activators of genes differentially regulated between LD-low and LD-high microglia. Remarkably, the pro-inflammatory endotoxin lipopolysaccharide (LPS) was the most significant upstream regulator, suggesting a link between innate inflammation and lipid droplets in microglia (FIG. 3, Panel F).

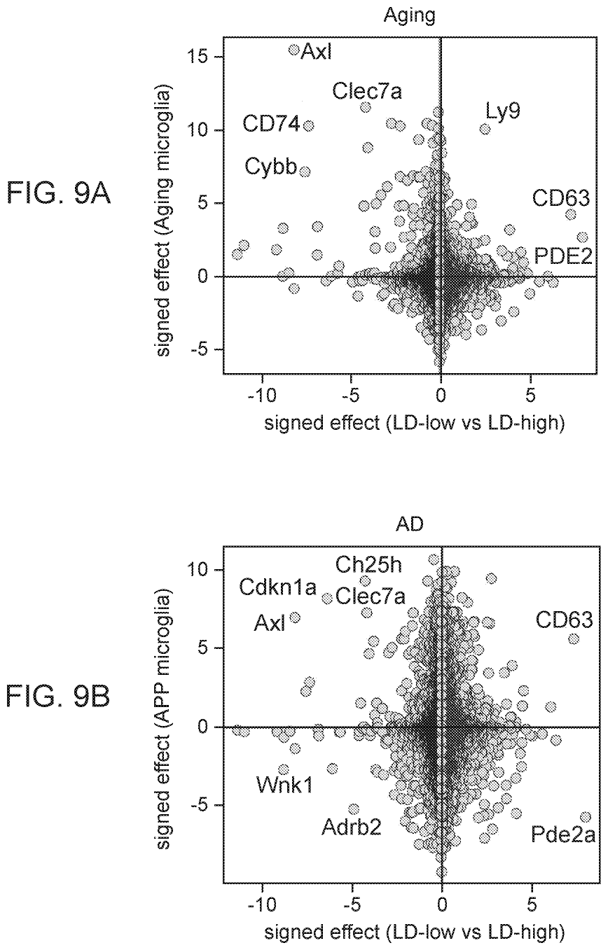

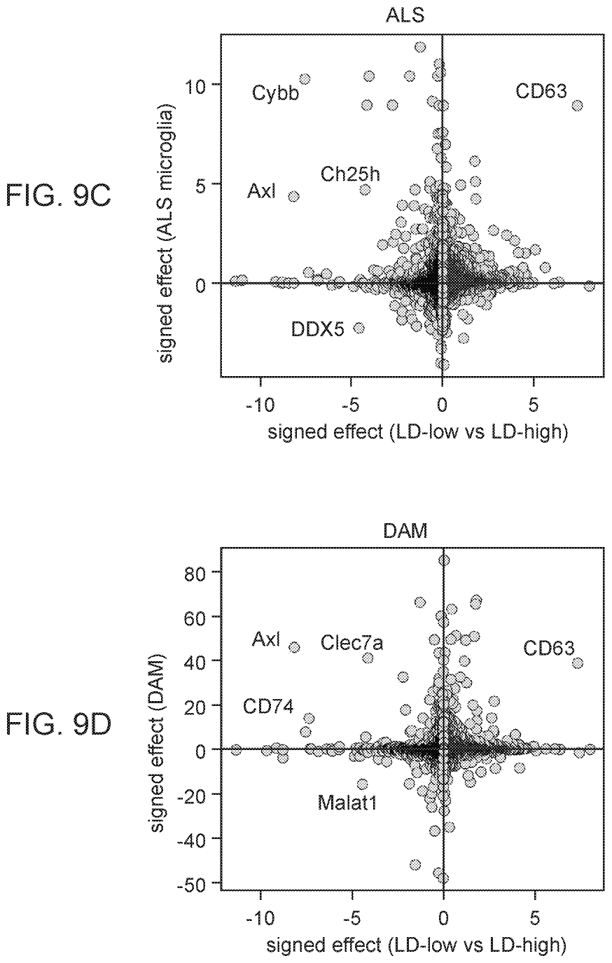

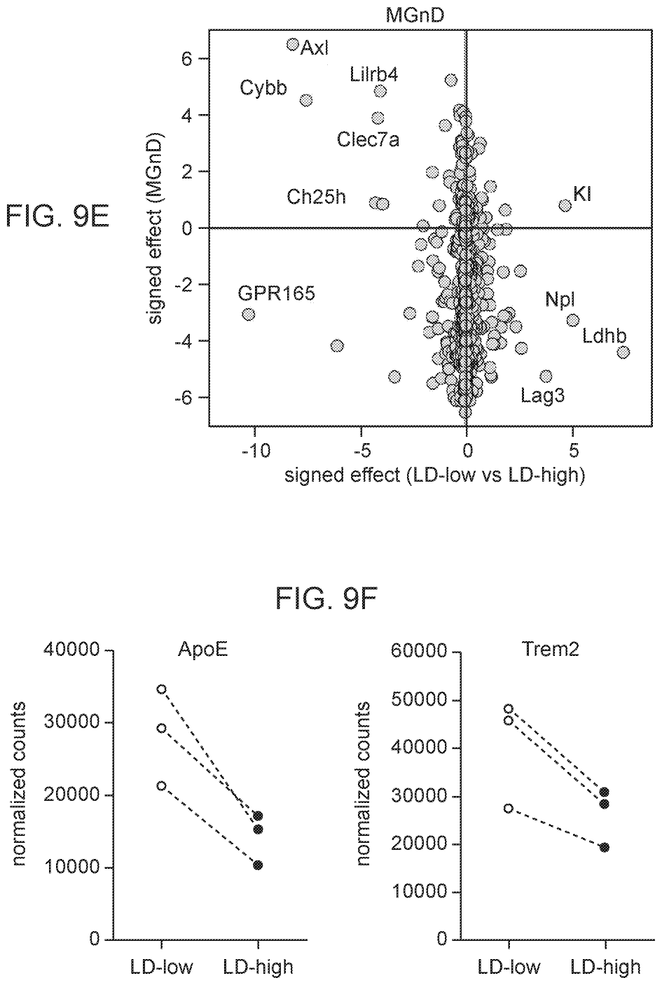

Lastly, the transcriptional signature of LD-high microglia was compared with the transcriptome of microglia in aging⁴⁰, ALS⁴¹, and AD⁴², as well as of the recently described “disease-associated microglia” (DAM)¹² and “neurodegenerative microglia” (MGnD)¹¹. It was found that genes that were downregulated in LD-high microglia overlapped partially with these published gene sets (FIG. 3, Panel G). However, the downregulated genes of LD-high microglia matched primarily with genes that were upregulated in microglia in aging, and in DAM (e.g. AXL, CD74, CLEC7A, CYBB). Furthermore, the TREM2 and APOE genes showed a trend for downregulation in LD-high microglia (FIG. 9, Panels A-F).

Overall, these data suggest that lipid droplet-containing aged microglia show transcriptional changes of genes related to key microglia functions such as phagocytosis, ROS production and immune signaling, yet they have a unique transcriptome signature that is distinct from previously described microglia states observed in aging and neurodegeneration. Therefore, this microglia state is referred to as “lipid droplet accumulating microglia” (LAM) herein.

### Example 3: Determining the Effect Lipopolysaccharide (LPS) Treatment on Lipid Droplet Concentration

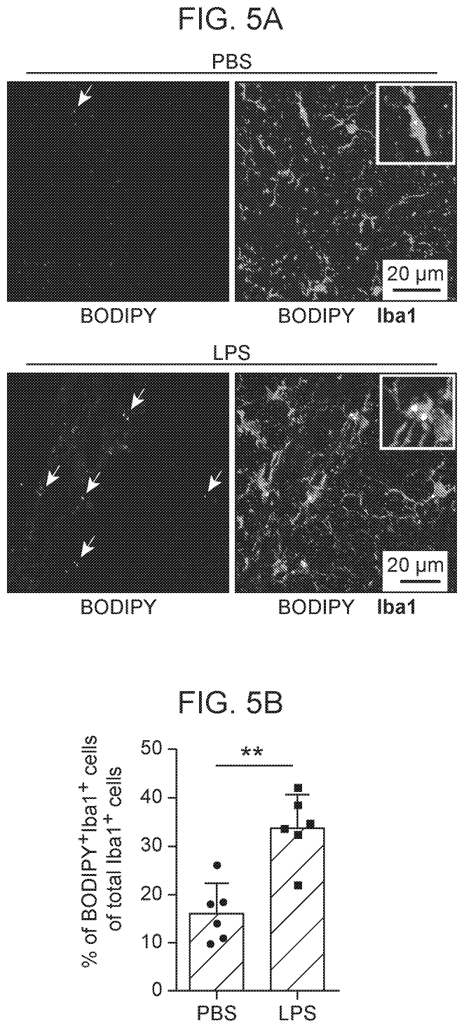

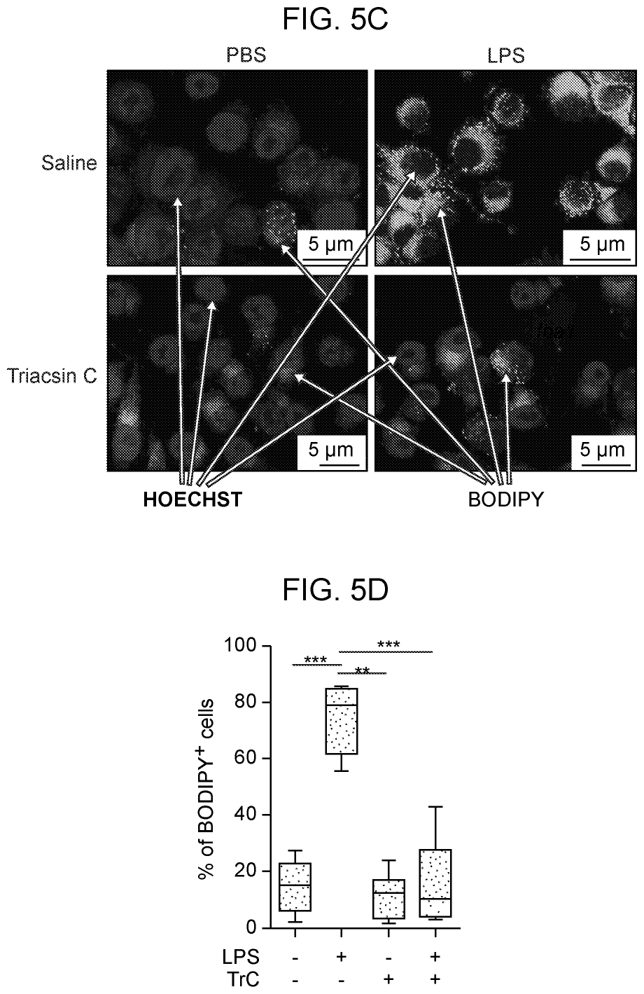

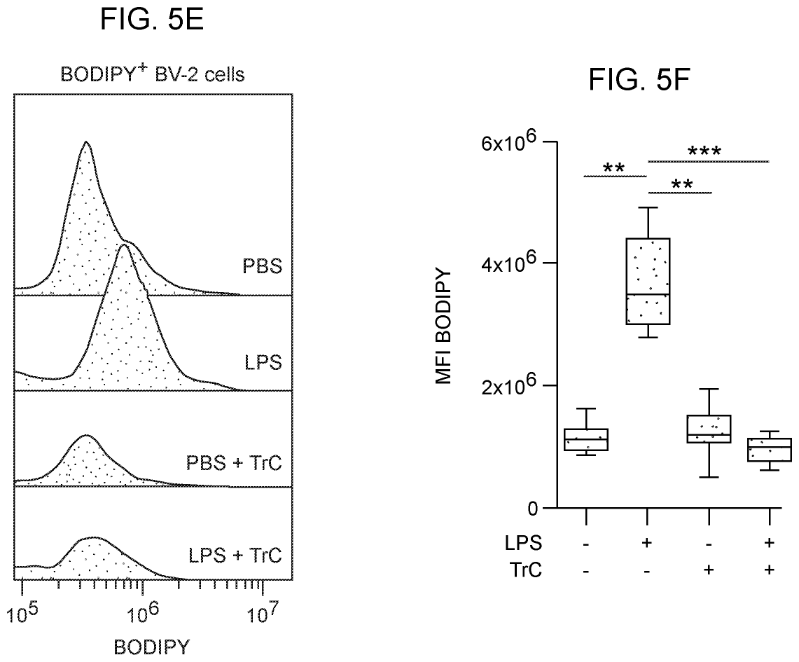

LPS is the main upstream regulator of genes differentially expressed between lipid droplet-low and lipid droplet-high microglia (FIG. 3, Panel F) and immune cells such as macrophages, neutrophils, and eosinophils accumulate lipid droplets in response to inflammatory conditions²⁰. Therefore young mice that showed low numbers of lipid droplet-containing microglia (FIG. 1, Panel A) were treated systemically with LPS at a dose that has been previously shown to induce a pro-inflammatory phenotype in microglia (1 mg LPS/kg BW for four days⁴⁶). Remarkably, a significant, twofold increase was observed in lipid droplet containing microglia (BODIPY⁺lba1⁺) in the hippocampus of LPS-treated mice compared with vehicle treated controls (FIG. 5, Panels A-B).

Likewise, stimulation of the mouse microglia-derived cell line BV-2 with LPS induced a 5-fold increase of lipid droplet containing BODIPY⁺BV-2 cells compared with control BV-2 cells (FIG. 5, Panels C-D), consistent with the effect of LPS in N9 microglia and brain slices^{33,35}. To confirm the identity of these BODIPY-labeled structures, Triacsin C was used, which is an inhibitor of long-chain acyl-CoA synthetase that inhibits de novo synthesis of triglycerides and prevents lipid droplet formation^{35,47}. Indeed, treatment with Triacsin C abolished the LPS-induced increase in BODIPY⁺ cells (FIG. 5, Panels C-D). Accordingly, LPS-treated BV-2 cells showed significantly increased BODIPY⁺ fluorescence when analyzed by flow cytometry (FIG. 5, Panels E-F), and co-treatment with Triacsin C inhibited an increase in BODIPY⁺ signal in LPS-stimulated BV-2 cells (FIG. 5, Panels E-F).

Together, these findings demonstrate that LPS triggers lipid droplet formation in microglia in vivo an in vitro, and that this model is useful for the study of LAM.

### Example 4: Analyzing Phagocytosis in Lipid Droplet Accumulating Microglia (LAM)

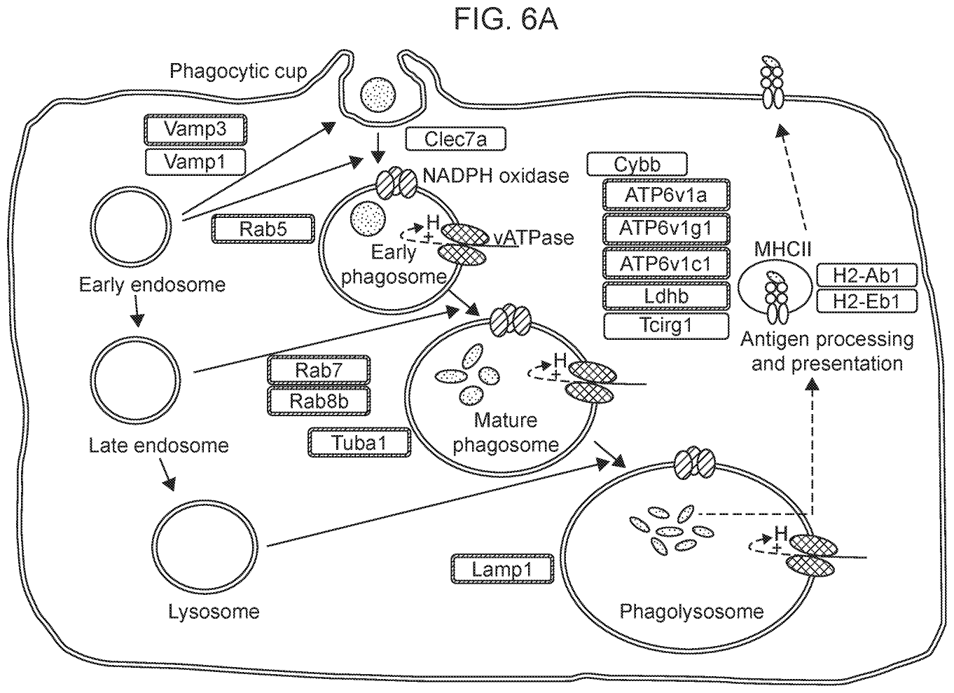

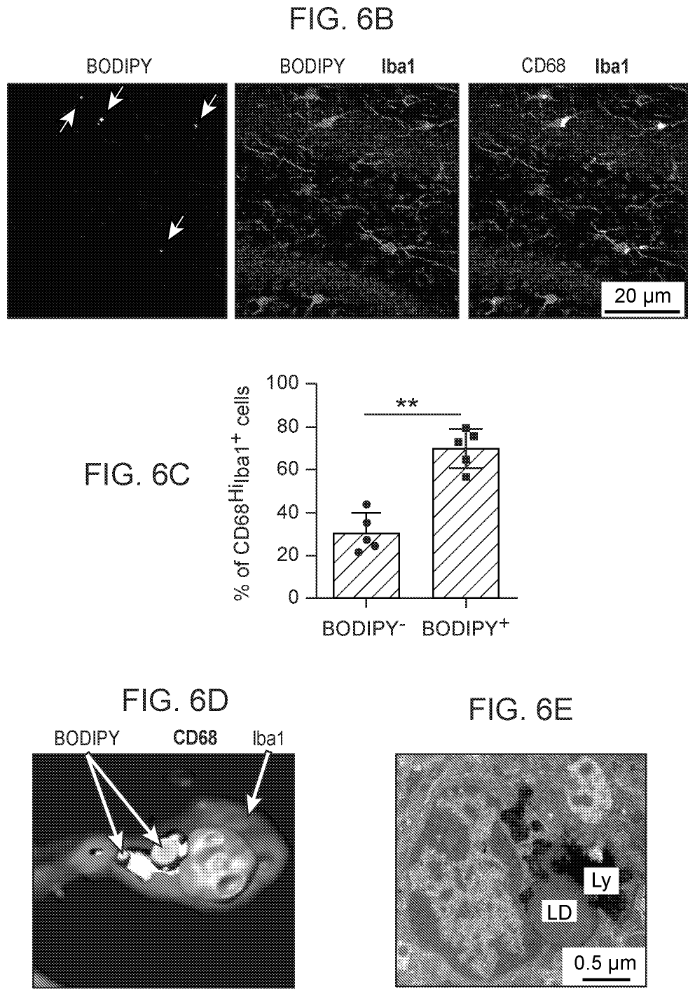

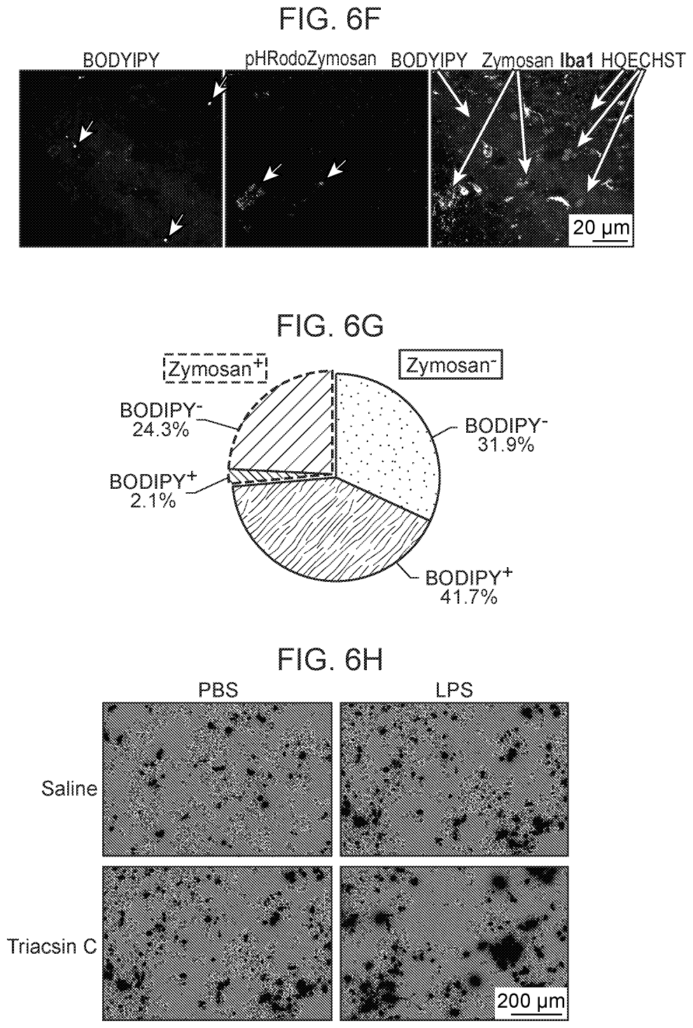

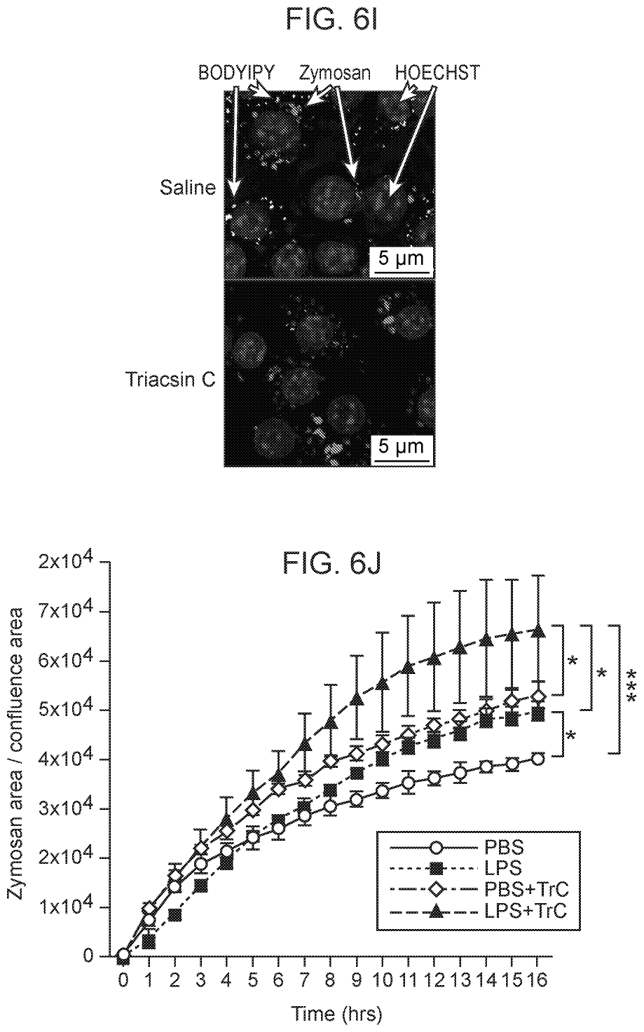

Because Phagosome maturation was the top regulated pathway associated with the transcriptome of LAM (FIG. 3, Panel E), it was examined whether the phagocytosis pathway was altered in these cells. Interestingly, RNA-Seq analysis revealed an upregulation of endosomal and lysosomal genes (FIG. 6, Panel A), suggesting that LAM show an increased load of endosomal/lysosomal vesicles. To corroborate this hypothesis, immunoreactivity was assessed for the lysosome-associated protein CD68 in in the hippocampus from aged mice and it was found that BODIPY⁺ microglia had significantly higher CD68 expression than BODIPY⁻ microglia (FIG. 6, Panels B-C). Furthermore, 3D reconstruction revealed that CD68+ vesicles often accumulate closely around lipid droplets (FIG. 6, Panel D). Electron microscopy confirmed an accumulation of lysosomes in lipid droplet containing microglia (FIG. 6, Panel E).

To analyze phagocytosis in LAM, acute organotypic brain slices were prepared from 12-month-old mice and treated with pHrodo red Zymosan particles. pHrodo Zymosan becomes fluorescent at an acidic pH and thus serves as a sensor for lysosomal degradation of phagocytosed material. The percentage of Zymosan⁺ BODIPY⁺ microglia was ten-fold lower compared to Zymosan⁺ BODIPY⁻ microglia (2.1% vs 24.3%), indicating that LAM have severe defects in degrading Zymosan (FIG. 6 Panels F-G).

Next, to determine if modulation of lipid droplets affects phagocytic activity, the in vitro model described earlier (FIG. 5, Panels C-D) was used to induce (LPS) and to inhibit (Triacsin C) lipid droplet formation in microglial BV-2 cells, and analyzed the pHrodo red Zymosan uptake in these cells (FIG. 6, Panels H-I). Indeed, Triacsin C increased Zymosan phagocytosis in LPS-treated cells (FIG. 6, Panel J). LPS itself significantly increased phagocytosis in BV-2 cells. High magnification images further revealed that Zymosan particles were mainly found in cells with low lipid droplet load (FIG. 6, Panel I). Collectively, these data suggest that increased lipid storage is associated with impaired phagocytosis.

### Example 5: Assessing ROS and Pro-Inflammatory Cytokine Levels in LAMs

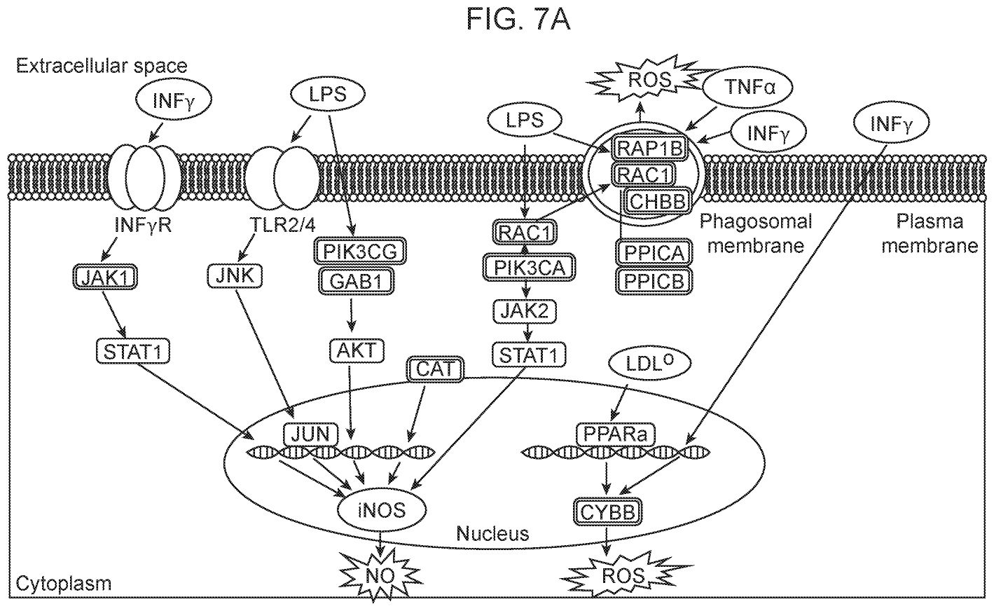

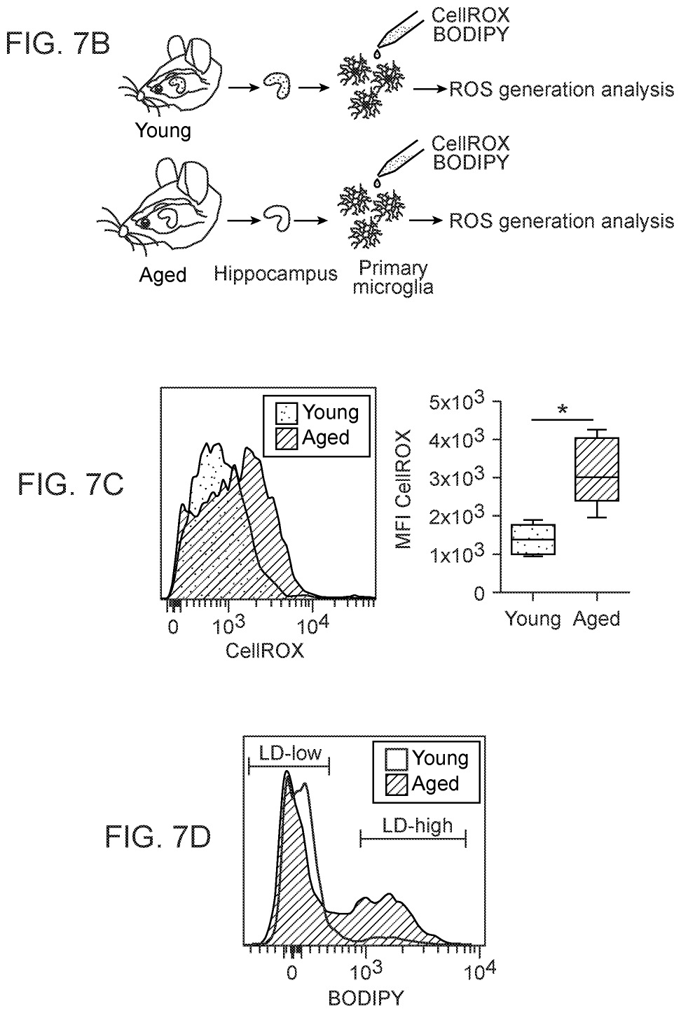

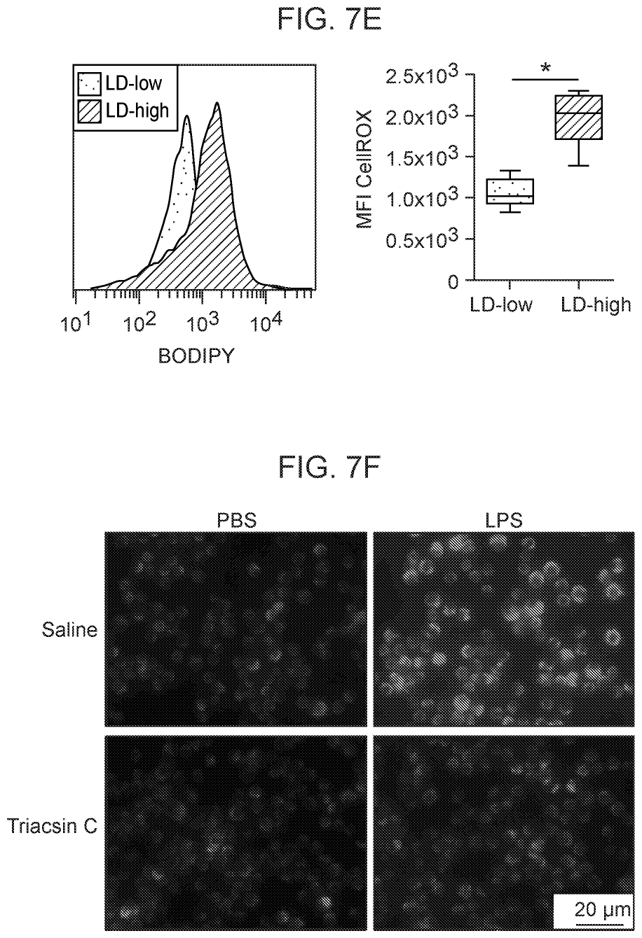

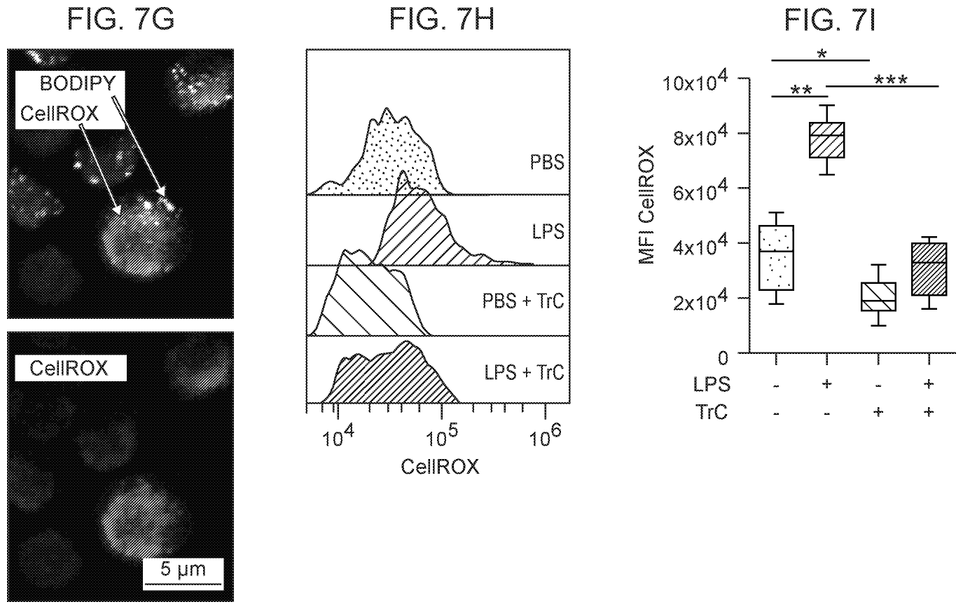

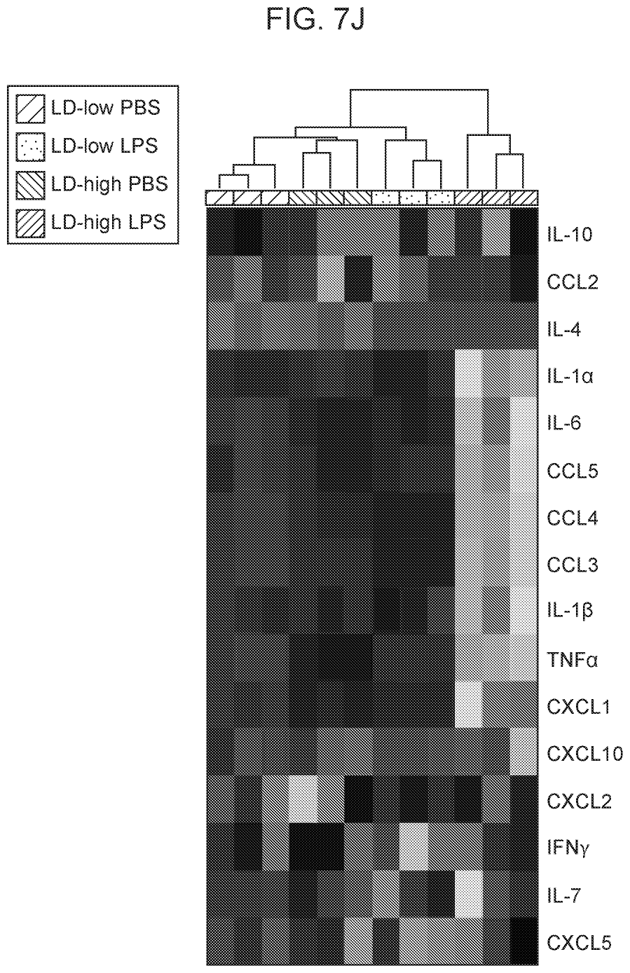

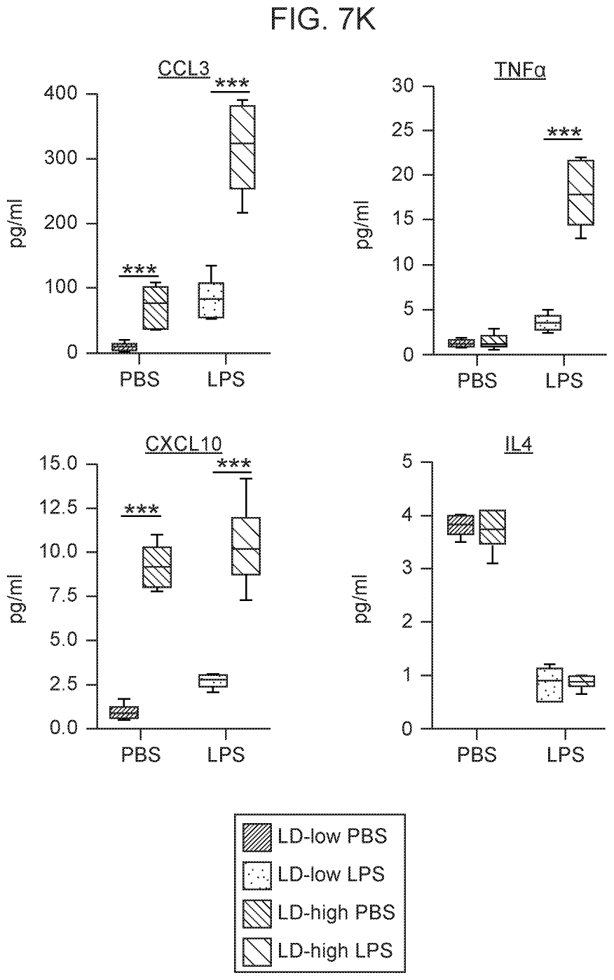

Pathway analysis of genes differentially expressed between aged LD-low and LD-high microglia identified Production of NO and ROS as the second most significantly regulated pathway (FIG. 3, Panel E). Given that over 90% of the differentially expressed genes related to ROS and NO production were upregulated (FIG. 7, Panel A), it was hypothesized that LAM generate increased levels of ROS.

First, it was tested whether ROS production was changed in aged compared to young microglia in general. Single cell suspensions were stained from the hippocampi of young and aged mice with CellROX, a dye that is non-fluorescent in a reduced state but exhibits bright fluorescence upon oxidation by ROS, and analyzed CellROX fluorescence within the CD11b⁺CD45^{lo} microglia population by flow cytometry (FIG. 7, Panel B). In line with previous reports⁵¹, we found that aged microglia had significantly higher ROS levels than young microglia (FIG. 7, Panel C).

Next, ROS levels were measured in LD-low and LD-high microglia from aged mice and it was found that CellROX signal was twofold increased in LD-high compared to LD-low microglia (FIG. 7, Panels D-E), suggesting that the elevated ROS levels in aged microglia might be specifically driven by the increased ROS generation of LAM.

To examine whether modulation of lipid droplet numbers would affect ROS production, LPS was used to induce lipid droplets in the microglial cell line BV-2 and inhibited lipid droplet formation by co-treatment with Triacsin C (FIG. 5, Panels C-D). CellROX fluorescence was assessed using microscopy and flow cytometry. In line with previous reports, LPS treatment induced elevated ROS generation in BV-2 cells⁵². Inhibition of lipid droplets with Triacsin C was sufficient to significantly reduce ROS levels in LPS-treated cells (FIG. 7, Panels F, H, I). Moreover, high magnification confocal imaging of LPS-treated BV-2 cells revealed that cells with high CellROX fluorescence were often loaded with lipid droplets (FIG. 7, Panel G). Together, these findings demonstrate that LAM have elevated concentrations of ROS and suggest that lipid droplets mediate LPS-induced ROS generation.

Another characteristic of aged microglia is the increased production of inflammatory cytokines such as TNF-α, IL-1β and IL-6 under baseline conditions and excessive cytokine release upon immune challenge. To determine the cytokine expression profile of LAM, hippocampal LD-low and LD-high microglia were acutely isolated from aged mice and cytokine concentrations were measured in the supernatant using multiplex array 8 h after stimulation with LPS or saline as a control. It was found that under baseline conditions (saline treatment), LD-high microglia released increased levels of multiple cytokines, including CCL3, CXCL10, IL-6, compared to LD-low microglia. Strikingly, LD-high microglia showed a strongly exaggerated release of multiple cytokines, such as IL-10, CCL3, CCL4, IL-6, CCL5, TNF-α, IL-1β, IL-1α, CXCL1 and CXCL10 upon LPS treatment compared to LD-low microglia (FIG. 7, Panels J-K). These findings suggest that LAM are in a primed activation state that becomes hyperactivated upon stimulation with LPS.

### Example 6: Screening for Genetic Regulators of Microglial Lipid Droplet Formation

To investigate genetic regulators of microglial lipid droplet formation, pooled CRISPR-Cas9 screens were performed. Informed by RNAseq data of LAM (FIG. 3, Panels C and E), screening with an sgRNA library was chosen that involved targeting ˜2000 genes involved in the lysosomal pathway and protein degradation as well as in cellular stress, with 10 distinct sgRNAs targeting each gene along with ˜1000 negative control sgRNAs⁵³. To probe the role of these genes in lipid droplet formation, the microglial BV-2 cell line was used to generate a pooled population of targeted BV-2 cells for every gene represented in the sgRNA library. LPS was used to induce lipid droplets in these cells (FIG. 5, Panels C-D).

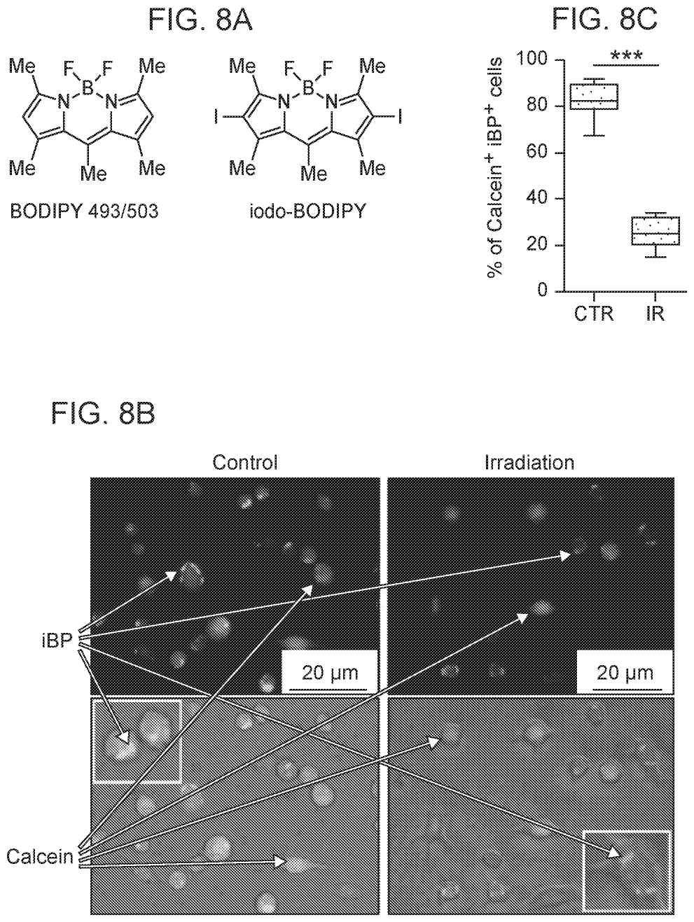

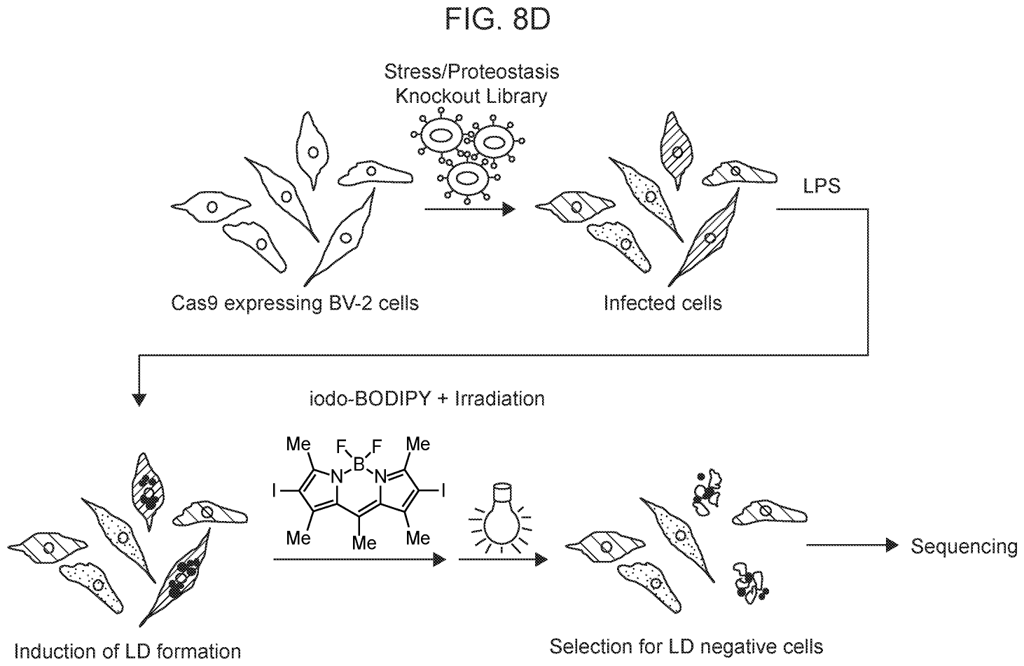

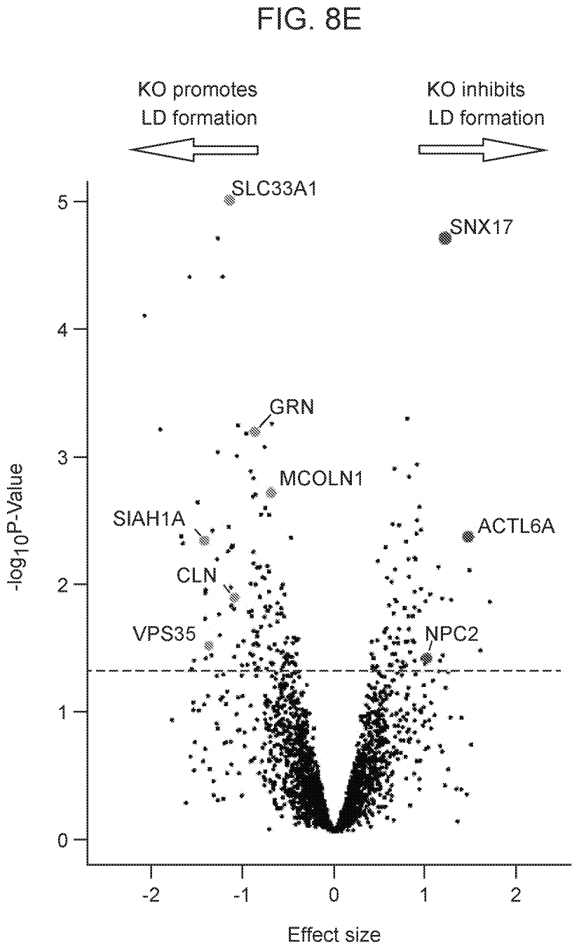

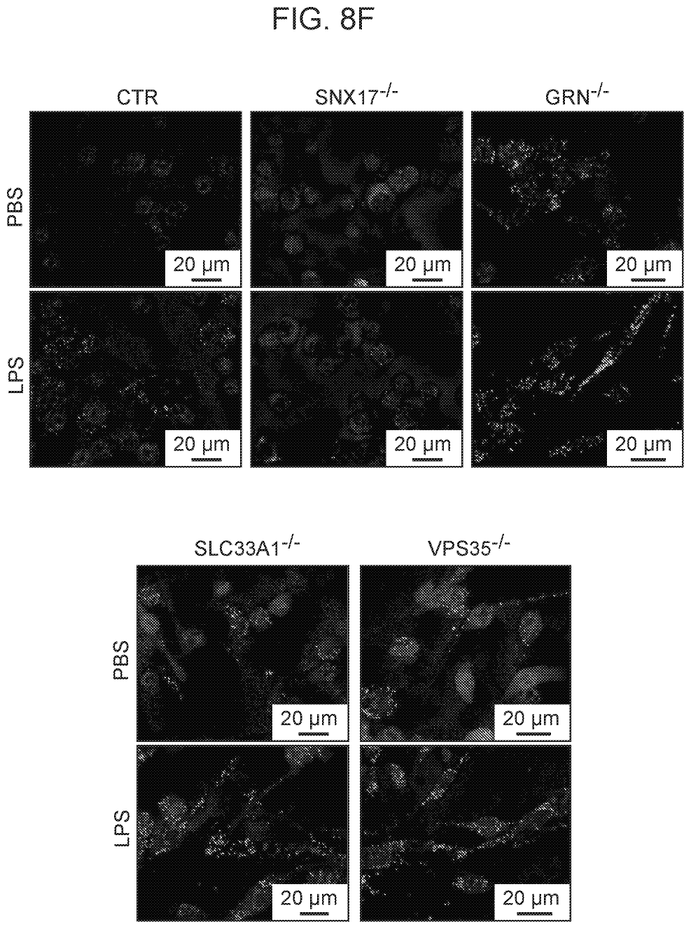

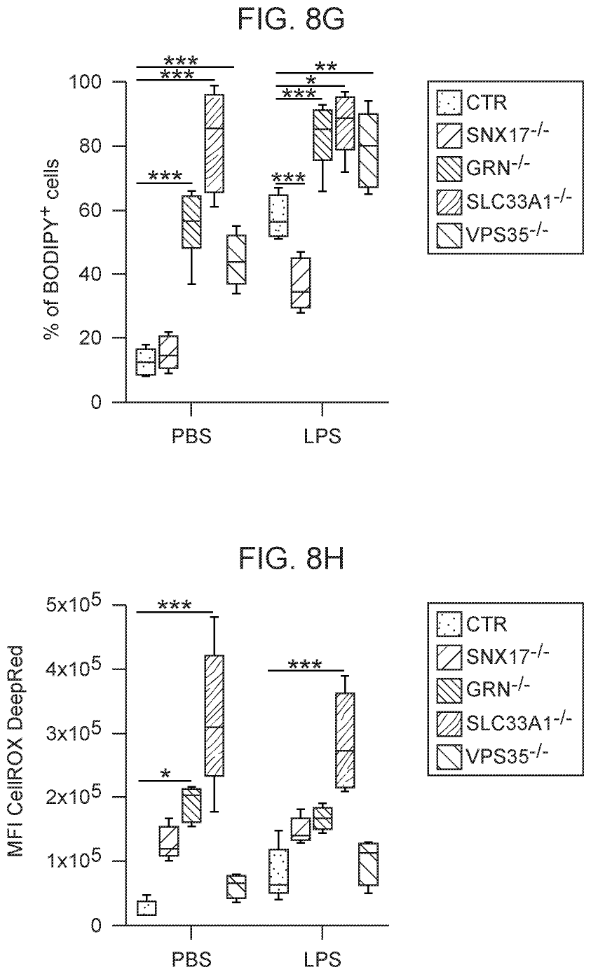

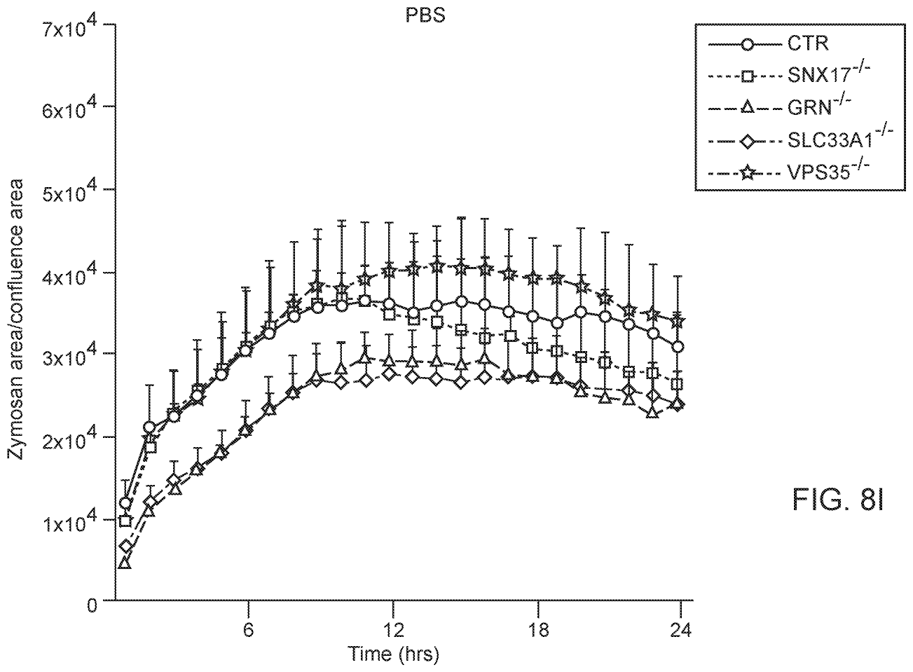

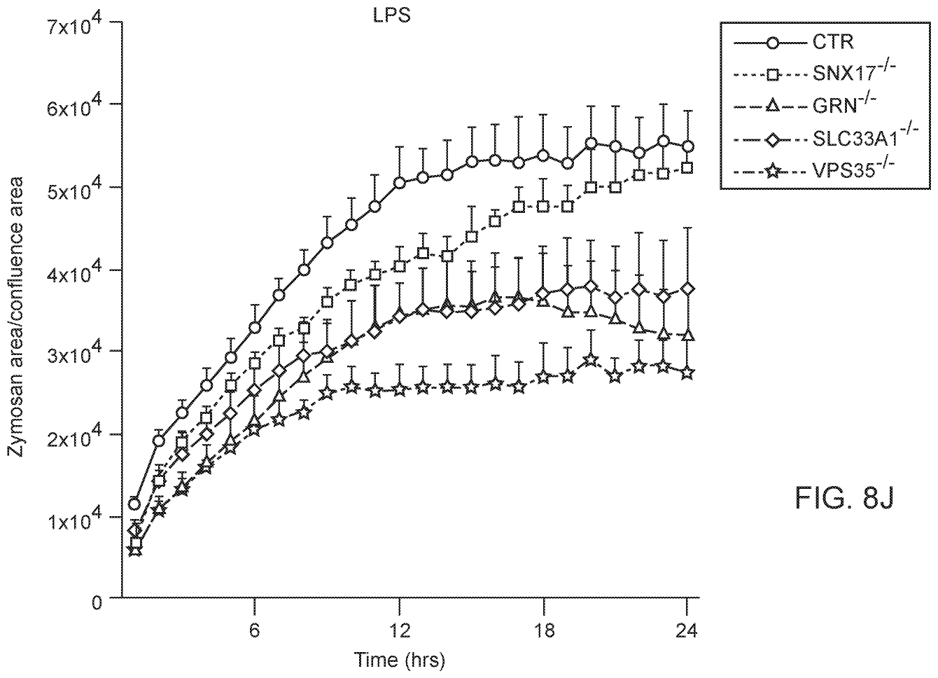

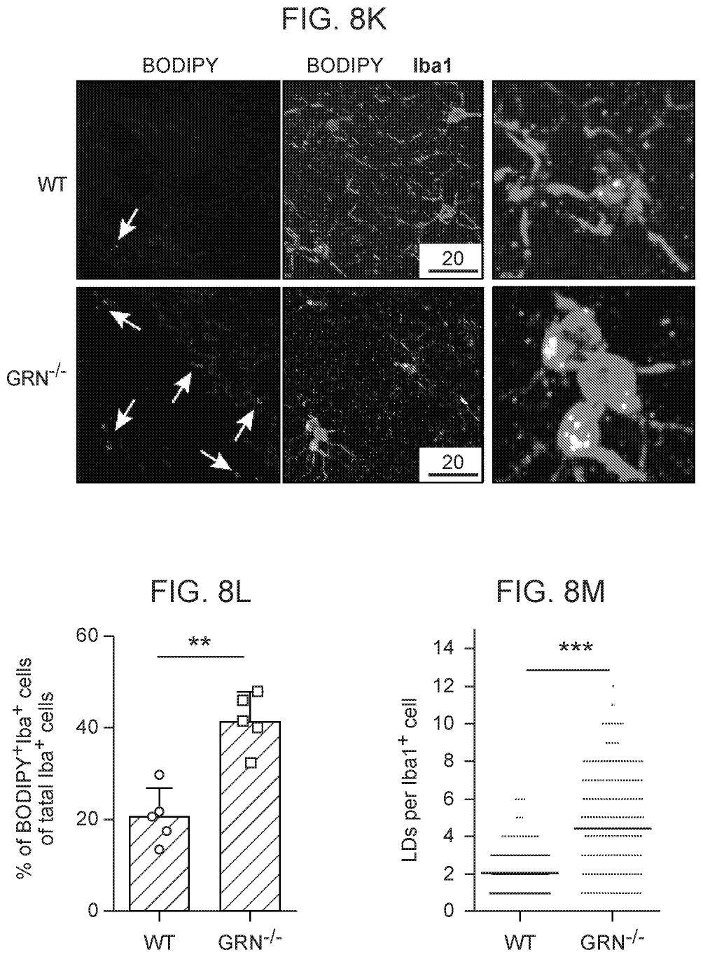

To identify sgRNAs that inhibited or promoted the formation of lipid droplets, a photoirradiation selection strategy was developed in which BV-2 cells were separated based on their capacity to form lipid droplets. By adding iodine atoms to the lipid droplet marker BODIPY(iodo-BODIPY, iBP; the synthesis followed the procedure described previously by Nagano and coworkers⁵⁴), this molecule was transformed into a photosensitizer that induces cell death in iBP⁺ cells after photoexcitation^{54,55} (FIG. 8 Panels A, D). To prove the efficacy of the photoablation approach, Calcein live cell imaging was used and it was found that irradiation of iBP⁺BV-2 cells selectively killed lipid-droplet rich cells (FIG. 8, Panels B-C).

After three rounds of selection against lipid droplet-rich cells, the sgRNA composition of the selected lipid droplet-negative BV-2 cells were sequenced. It was found 112 genes that were significant positive or negative regulators of lipid droplet formation (P<0.05; FDR<50%; FIG. 8, Panel E). The top hits included the SLC33A1, SNX17, GRN and VPS35 genes, suggesting a possible relationship between lipid storage in microglia and neurodegeneration.

Individual BV-2 cell lines were generated with CRISPR deletions of selected hits that were detected as negative (SLC33A1, GRN, VSP35) or positive (SNX17) regulators of lipid droplet formation in the screen. Indeed, BV-2 cells with sgRNAs targeting GRN, SLC33A1 and VPS35 had significantly more lipid droplets than BV-2 cells with control sgRNAs, and sgRNAs targeting SNX17 inhibited lipid droplet formation upon LPS treatment (FIG. 8, Panels F-G). Flow cytometry was used to measure ROS levels and it was found that ROS levels significantly increased in BV-2 cells with sgRNAs targeting GRN and SLC33A1 cells under baseline conditions and in SLC33A1 sgRNA expressing cells upon LPS treatment (FIG. 8, Panel H). Next, phagocytosis was assessed by analyzing pHrodo red Zymosan uptake and it was found that cells with sgRNAs targeting GRN and SLC33A1 had significant deficits in Zymosan uptake compared to control cells. In VPS35 sgRNA expressing cells, phagocytosis was specifically compromised upon LPS treatment but not under baseline conditions. BV-2 cells expressing sgRNAs against SNX17 did not show impairments in phagocytosis (FIG. 8, Panels I-J).

Finally, to confirm the findings from the screen in vivo, lipid droplet numbers were analyzed in microglia in GRN^{-/-} mice. Given the results of the CRISPR-Cas9 KO screen, it was hypothesized that the GRN KO would promote lipid droplet formation. It was found that microglia in the hippocampus of 9-month old GRN^{-/-} mice are indeed loaded with high amounts of lipid droplets, resulting in a more than twofold increase of lipid droplet numbers per cell in GRN^{-/-} mice compared with age-matched wild type controls (FIG. 8, Panels K and M). In addition, the percentage of BODIPY⁺ microglia was twofold higher in GRN^{-/-} mice than in wild type mice (FIG. 8, Panel I).

Experimental Details

Animals

Aged C57BL/6J male wild type mice (18-20 months old) were obtained from the National Institute on Aging (NIA), and young C57BL/6J males (2-4 months of age) were purchased from Jackson Laboratory. 9-month old male wild type and Grn^{-/-} deficient mice (B6.129S4(FVB)-Grntm1.1Far/Mmja) were bred and aged in-house but originally acquired from Jackson. Mice were housed under a 12-hour light-dark cycle in pathogenic-free conditions, in accordance with the Guide for Care and Use of Laboratory Animals of the National Institutes of Health.

LPS Injections

3-month-old wild type mice were injected with LPS (Lipopolysaccharide from E. coli, Sigma), i.p. 1 mg LPS/kg body weight once daily for four consecutive days. Control mice were injected with body weight corresponding volumes of PBS. 24 hours after the last LPS injection mice were euthanized, brains were extracted and brain tissue was processed for immunohistochemistry staining (see Perfusion and tissue processing and Immunohistochemistry and BODIPY staining).

Electron Microscopy

3-month and 20-month-old C57BL/6 wild type mice (n=3 per group) were anesthetized with 3.8% chloral hydrate (wt/vol) and transcardially perfused with 0.9% saline, followed by 2% paraformaldehyde/2.5% glutaraldehyde in 0.1M phosphate buffer (PB; pH7.4). Brains were removed and postfixed in the same fixative overnight (4° C.) and then stored in 0.1 M PB. Sagittal 200 μm sections were cut on a Leica VT1000S vibratome (Leica). The sections were incubated in 2% osmium (Electron Microscopy Sciences) for 2 h, rinsed in 0.1M PB, dehydrated in a graded series of ethanol and embedded in Araldite (Durcupan, Electron Microscopy Sciences). Semithin (1.5 μm) and ultrathin (70-80 nm) sections of the dentate gyrus were cut using a Reichert Om-U 3 ultramicrotome (Leica). Ultrathin sections were mounted on Formvar coated 75-mesh copper grids, contrasted with aqueous solutions of uranyl acetate (0.5%) and lead citrate (3%), and analyzed at 80 kV in a EM 910 transmission electron microscope (Zeiss) equipped with a Troendle sharp:eye 2k CCD camera.

To evaluate the ultrastructure of microglia within the dentate gyrus, 10 ultrathin sections were analyzed per mouse. Microglia were identified by a combination of ultrastructural characteristics, including a highly electron-dense cytoplasm and nucleus, an often star-shaped cell morphology, an irregularly shaped nucleus with coarsely clumped chromatin, and a cytoplasm rich in free ribosomes and vesicles¹. Cytoplasm and nucleus area were analyzed using ImageJ software 1.45s.

Perfusion and Tissue Processing

Mice were anesthetized using Avertin (Tribromoethanol) and transcardially perfused with 0.9% NaCl solution. Brains were extracted, fixed in 4% paraformaldehyde for 48 hours, cryoprotected in 30% sucrose and then sectioned sagitally or coronally (40 pm) using a freezing microtome (Leica). Sections were stored at −20° C. in cryoprotectant solution (ethylene glycol, glycerol, 0.1 M phosphate buffer pH 7.4, 1:1:2 by volume) until used for immunohistochemistry and CARS imaging.

Immunohistochemistry and BODIPY Staining

Free-floating sections were washed three times in PBS, followed by 1 hour blocking in PBS with 10% donkey serum. Sections were incubated in PBS with 10% donkey serum and primary antibodies for 48 hours at 4° C.: rabbit anti-TMEM119 (1:400, Abcam, ab209064), rat anti-CD68 (1:200, Bio-Rad, MCA1957GA), rabbit anti-lba1 (1:1000, Wako, 019-19741). After the primary antibody incubation, sections were washed three times in PBS and incubated in PBS with 10% donkey serum and secondary antibodies for 3 hours at room temperature (RT): donkey anti-rabbit Alexa 555, donkey anti-rabbit Alexa 647 (all 1:500, Invitrogen), donkey anti-rat Cy5 (1:500, Jackson Immuno Research). Sections were washed once in PBS and incubated in PBS with BODIPY™ 493/503 (1:1000 from 1 mg/ml stock solution in DMSO; ThermoFisher) to stain lipid droplets and Hoechst 33342 (1:2000, ThermoFisher) for nuclear counterstaining for 15 min at RT. Sections were mounted on microscope slides and embedded with Vectashield (H-1000, Vector Laboratories). Note that for successful lipid droplet staining, antigen retrieval steps and treatment with detergents have to be avoided, and sections should be embedded while still wet.

Quantitative Analysis of Immunohistological Data

For quantification of BODIPY⁺ microglia, of CD68^{hi} microglia, of BODIPY⁺ lipid droplet numbers and of lipid droplet size, analyses were performed blinded on coded slides. Every tenth section (400 pm interval) of one hemisphere was selected from each animal and processed for immunohistochemistry. Six randomly selected visual fields per animal were photographed using a confocal scanning laser microscope (LSM 700, Zeiss) with LSM software (ZEN 2011).

To analyze the percentage of lipid droplet-containing microglia, numbers of total TMEM119⁺ or lba1⁺ cells and of TMEM119+BODIPY⁺ or lba1+BODIPY⁺ cells were counted and the percentage of BODIPY⁺ microglia was calculated. To assess the percentage of CD68^{hi} cells, BODIPY⁻lba1⁺ and BODIPY⁺lba1⁺ cells with clearly visible CD68 immunoreactive particles were counted and normalized to total BODIPY⁻lba1⁺ and BODIPY⁺lba1⁺ cells.

To assess the numbers of lipid droplets per dentate gyrus area, BODIPY⁺ lipid droplets from six randomly selected visual fields (maximum projection of the z-stack across the whole section) were manually counted and normalized to the corresponding dentate gyrus area. To determine the average size of lipid droplets, BODIPY⁺ signal was analyzed using the ‘Analyze Particles’ function of ImageJ 1.45 s (ImageJ website: http://imagej.nih.gov.laneproxy.stanford.edu/ij/) for six randomly selected visual fields (maximum projection of the z-stack across the whole section).

3D Reconstruction of Confocal Images

Confocal image stacks (acquired at 63× magnification) of BODIPY⁺ and CD68⁺ microglia were converted to 3D images with the surface-rendering feature of Imaris BitPlane software (version 7.6.1).

Human Post-Mortem Brain Tissue

Human hippocampal tissue sections from autopsy samples of young adult (<35 years, n=3) and elderly (>60 years, n=5) humans with a post-mortem interval <24 h were used. Human post-mortem tissue was obtained from the collection of the Department of Neuropathology at Stanford University. The use of these specimen for scientific purposes was in accordance with institutional ethical guidelines. All samples used were obtained from individuals without any neurological or psychiatric diagnoses. After tissue extraction, the brain samples were stored in 10% formalin. All tissue samples were cut at 50 pm on a vibratome (Leica VT1000S) and stored in PBS at 4° C.

Immunohistochemistry of Formalin Fixed Human Brain Tissue

Because immunostaining of formalin-fixed human brain samples requires antigen retrieval steps and the use of detergents (see methods for detailed protocol) which leads to the removal of lipids from tissue, we were unable to use the neutral lipid stain BODIPY. Instead we used an antibody against the lipid droplet surface protein Perilipin2 (Plin2) to detect lipid droplets in human postmortem hippocampus of adult and aged cognitively normal individuals. Formalin-fixed human tissue sections were washed three times in Tris-Buffered Saline with 0.05% Tween 20 (TBST), followed by incubation in TBST with 10% donkey serum and primary antibodies for 72 hours at 4° C.: rabbit anti-lba1 (1:500, Wako, 019-19741), guinea pig anti-Adipophilin (Plin2) (1:200, Fitzgerald, 20R-AP002). After the primary antibody incubation, sections were washed three times in TBST and incubated in TBST with 10% donkey secondary and secondary antibodies for 3 hours at RT: donkey anti-rabbit Alexa 488, donkey anti-guinea pig Alexa 555 (all 1:500, Invitrogen). Sections were mounted with Vectashield (H-1000, Vector Laboratories).

CARS Imaging

PFA-fixed 40 pm thick brain sections from 3-month and 20-month old mice (n=5 mice per group; 4 sections per animal) were stained for rabbit anti-TMEM119 (1:400, Abcam, ab209064) and donkey-anti rabbit Alexa 647 (Jackson Immuno Research) (Immunohistochemistry protocol see above). Microscopy was performed using a Leica SP5 confocal microscope with spectral detection (Leica Microsystems Inc., Germany) and a Leica HCX PL APO CS 63× NA 1.4 oil immersion objective. Alexa 647 was excited at 633 nm and emission detected between 650-700 nm using a hybrid detector (HyD). Transmission images were acquired simultaneously. Label-free coherent anti-Stokes Raman scattering (CARS) microscopy was performed using a commercial setup consisting of an optical parametric oscillator pumped by a picosecond laser source (picoEmerald; APE, Germany) integrated into the Leica SP5 microscope. The CARS signal was detected using a 650/210 bandpass emission filter and a non-descanned detector (NDD) in epi-mode. To detect neutral lipids the laser was tuned to 2845 cm⁻¹, thus enabling imaging of CH₂ symmetric stretching vibrations. CARS and fluorescence/transmission images were acquired sequentially. For quantification of CARS signal in microglia, 20 randomly selected microglia in the dentate gyrus per animal were imaged. The percentage of TMEM119⁺ microglia with CARS⁺ vesicles from total TMEM119⁺ microglia was calculated.

Lipidomics

5-month and 20-month old wild type mice (n=4 mice/group) were perfused and hippocampus and liver were extracted. Alternatively, hippocampal microglia were FACS-sorted from 20-month old mice (see “Microglia Isolation” above). Lipid droplets from liver, whole hippocampus, and isolated microglia were isolated using the lipid droplet isolation kit from Cell Biolabs according to the manufacturer's instructions.

Lipid droplets were stored at −70° C. until sample preparation and extracted according to a modified Bligh & Dyer protocol² and as reported earlier⁷. Prior extraction, 10 μL of a synthetic lipid standard mastermix (including 15 deuterated lipids) were added to 90 μL of extraction buffer containing lipid droplets.

Extracted lipids were analyzed by flow injection analysis (FIA) shotgun lipidomics using an ekspert™ MicroLC 200 system (eskigent, Singapore) hyphenated to a TripleTOF® 4600 System (AB SCIEX, Darmstadt, Germany) as reported earlier⁸. Each sample was injected twice, one for measurement in positive and one for negative ionization mode. Instrumental controlling and data acquisition was achieved with Analyst® TF Software (v 1.7, AB SCIEX, Darmstadt, Germany).

Data were processed with Lipid View™ software (v 1.3 beta, AB SCIEX, Darmstadt, Germany). Lipid identification was based on precursor ion and neutral loss scans specific for proposed lipid species. Internal standard correction for each lipid was carried out by normalization against the appropriate synthetic isotopically labeled lipid standard.

Microglia Isolation Primary microglia were isolated as previously described³. 18-month-old wild type mice were perfused with Medium A (HBSS+15 mM HEPES+0.05% glucose+1:500 DnaseI), and hippocampi were dissected (n=9 mice; 3 pooled hippocampi per sample). Hippocampi were chopped and homogenized using a Dounce homogenizer in 2 ml cold Medium A (HBSS+15 mM HEPES+0.05% glucose+1:500 DnaseI), filtered through 100 pm cell strainer, rinsed with 5 ml Medium A and centrifuged at 340 g for 5 min. For myelin removal, the precipitate was resuspended in 30% standard isotonic percoll (30% Percoll in PBS, diluted with Medium A) and centrifuged at 900 g for 20 min. Precipitated cells were washed with HBSS and resuspended in FACS buffer (PBS+1% BSA+2 mM EDTA). The samples were stained with 1:300 CD11b-PE and 1:300 CD45-APC for 30 min at RT, centrifuged at 400 g for 5 min, resuspended in PBS with BODIPY 493/503 (1:2000 from 1 mg/ml stock solution in DMSO; ThermoFisher) and incubated for 10 min at 37° C. Cells were washed two times with FACS buffer and resuspended in FACS buffer with DNAse1 and 5 μl/ml RNAse Inhibitor (Clontech). Dead cells were excluded by staining with Sytox blue dead cell stain (1:10,000, Invitrogen). Cells were isolated using an ARIA 3.1 (BD Biosciences) with FACSDiva software (BD Biosciences), were sorted into RLT lysis buffer (QIAGEN) with 1% 2-Mercaptoethanol and frozen at −80° C.

RNA Isolation and Library Preparation

Frozen cells were thawed to room temperature and total RNA was isolated from the cell pellets using the RNeasy Plus Micro kit (Qiagen, 74034). RNA quantities and RNA quality was assessed using the Agilent 2100 Bioanalyzer (Agilent Technologies). All samples passed a quality control threshold (RIN≥9.0) to proceed to library preparations and RNAseq. Total mRNA was transcribed into full length cDNA using the SMART-Seq v4 Ultra Low Input RNA kit from Clontech according to the manufacturer's instructions. Samples were validated using the Agilent 2100 Bioanalyzer and Agilent High Sensitivity DNA kit. 150 pg of full length cDNA was processed with the Nextera XT DNA library preparation kit from Illumina according to the manufacturer's protocol. Library quality was verified with the Agilent 2100 Bioanalyzer and the Agilent High Sensitivity DNA kit. Sequencing was carried out with Illumina HiSeq 2000/2500, paired end, 2×100 bp depth sequencer. The quality of fastq files was assessed using FASTQC (v 0.11.4). Reads were mapped to mouse mm9 reference genome using STAR (v 2.5.1b). Raw read counts were generated with STAR using the GeneCounts function.

RNA-Seq Differential Expression

Differential expression in RNA-Seq was analyzed using the R DESeq2 package (Love et al., 2014). Read counts were used as input and normalized using built-in algorithms in DESeq2. Pairwise comparisons among the two groups (BODIPY^{lo} and BODIPY^{hi} microglia) were done on all genes and 12129 genes with calculable fold changes (FC) and false discovery rates (fdr) were used for further analysis. False discovery rate was estimated using Benjamini and Hochberg approach (Benjamini and Hochberg, 1995). R was used for RNA-Seq data visualization and Ingenuity Pathway Analysis (IPA) was used to analyze pathways and upstream regulators.

For comparisons of transcriptome changes in LAM with published datasets (FIG. 3, Panel G and FIG. 9), we selected the following published RNA-seq datasets of microglia in aging and neurodegeneration (modified after Bohlen et al. 2017): 24-month versus 4-month old wild type mice for Aging (Holtman et al. 2015); APP+ versus APP− for AD (Wang et al., 2015); SOD^{G93A} endstage versus non-transgenic day 130 for ALS (Chiu et al., 2013); DAM versus homeostatic microglia (Keren-Shaul et al. 2017), MGnD versus homeostatic microglia (Kraseman et al. 2017). For gene set comparisons, we generated lists for the published datasets by using a 4-fold change cutoff (modified after Bohlen et al. 2017) and compared these lists with the top 100 up- and downregulated genes in LAM.

BV-2 Cell Culture

Cells from the murine microglial cell line BV-2 were originally obtained from Banca Biologica e Cell Factory, IRCCS Azienda Ospedaliera Universitaria San Martino, Genua, Italy. Cells were maintained in Dulbecco's Modified Eagle's Medium (DMEM, Life Technologies) supplemented with 10% fetal bovine serum (FBS) and antibiotics (penicillin 100 U ml⁻¹, streptomycin 100 U ml⁻¹, HVD Life Sciences) (Pen/Strep) under standard culture conditions (95% relative humidity with 5% CO₂ at 37° C.). Adherent cells were split using 1× TrypLE (GIBCO).

LPS and Triacsin C Treatment

To induce lipid droplet formation, subconfluent BV-2 cells were treated for 18 hours with 5 μg/ml LPS (Lipopolysaccharide from E. coli, Sigma) in DMEM+5% FBS. Controls received vehicle solution (PBS) only. To inhibit lipid droplet formation, BV-2 cells were pre-treated with 1 μM Triacsin C (Cayman Chemical) or vehicle (saline) in DMEM+5% FBS. 30 min after adding Triacsin C, LPS or LPS-vehicle solution were added and cells were co-treated with Triacsin C and LPS for 18 hours.

BODIPY In Vitro Staining

BV-2 cells were seeded at 5×10⁴ cells on poly-L-lysine coated glass coverslips in DMEM+5% FBS. Following specific treatments, cells were fixed in 4% PFA for 30 min, washed 3× in PBS and incubated in PBS with BODIPY™ 493/503 (1:1000 from 1 mg/ml stock solution in DMSO; ThermoFisher) and Hoechst 33342 (1:2000, ThermoFisher) for 10 min at RT. Sections were washed twice in PBS and mounted on microscope slides with Vectashield (H-1000, Vector Laboratories). Four randomly selected visual fields per coverslip were photographed (40× magnification) using a confocal scanning laser microscope (LSM 700, Zeiss) with LSM software (ZEN 2011). To analyze the percentage of lipid droplet-containing BV-2 cells, numbers of total Hoechst⁺ cells and of Hoechst⁺ cells with BODIPY⁺ lipid droplets were counted and the percentage of BODIPY⁺BV-2 cells was calculated.

In Vitro Phagocytosis Assay

For in vitro phagocytosis assays, BV-2 cells were split into 96 well plates at 1000 cells per well in DMEM+5% FBS and treated with LPS, Triacsin C and vehicle solutions for 18 hours. Following specific treatments, 5 ng pHRodo Red Zymosan™ Bioparticles (Thermo Fisher Scientific P35364) in 100 μl DMEM+5% FBS were added per well. Four phase contrast and red fluorescent images per well were acquired every 2 hours for 16 hours using the Incucyte S3 live cell analysis system (Essen Bioscience). For each time point, phagocytosis was calculated by normalizing red fluorescent area to phase confluence.

Organotypic Brain Slices and In Situ Phagocytosis Assay

12-month-old male wild type mice (n=3 mice) were decapitated and dissected brains were immediately put in pre-cooled culture medium with serum (65% MEM (Sigma); 10% horse serum; 25% HBSS; 6.5 mg/ml glucose; 2 mM Glutamine; 1% Pen/Strep).

The entire procedure was done on ice with pre-cooled solutions until culturing. Coronal sections were prepared with a vibratome (Leica VT1000S) at 250 μm thickness and then transferred to insert wells (Millicell Cell Culture Insert, 30 mm, Millipore) on a 6-well plate with medium. Sections were incubated for 1 hour in the incubator (37° C., 5% CO₂). pHrodo™ Red Zymosan Bioparticles (Thermo Fisher Scientific) were opsonized (3 washes in PBS, followed by incubation in 50% FBS in PBS for 45 min at 37° C. and three washes in PBS) and added at 0.5 mg/ml to cover the whole sections (about 150 μl each). The plate was incubated for 4 hours (37° C., 5% CO2). After washes with PBS, sections were fixed with 4% PFA for 30 min at RT. Immunohistochemistry for lba1 and BODIPY staining was performed as described above.

To measure phagocytosis in BODIPY⁻ and BODIPY⁺ microglia, four randomly selected visual fields per section (three sections per mouse) were photographed using a confocal scanning laser microscope (LSM 700, Zeiss) with LSM software (ZEN 2011). The numbers of BODIPY⁻lba1⁺ and BODIPY⁺lba1⁺ cells were quantified, and the percentage of Zymosan containing cells was calculated.

ROS Assays

To assess ROS generation in primary microglia, cell homogenates from 3- and 20-month-old mice were prepared and antibody staining was performed as described above (see Microglia Isolation). Cell homogenates were incubated in FACS buffer with CellROX™ Deep Red (1:500, Invitrogen) for 30 min at 37° C., washed twice in FACS buffer, and CellROX™ Deep Red Intensity was analyzed on ARIA 3.1 (BD Biosciences).

To measure ROS in BV-2 cells, cells were split into 24 well plates at 5×10⁴ cells per well in DMEM+5% FBS and treated with LPS, Triacsin C and vehicle solutions for 18 hours. Next, cells were incubated in DMEM+5% FBS with CellROX™ Orange (1:500, Invitrogen) for 30 min at 37° C., washed twice in PBS, and CellROX™ Orange Intensity was examined by fluorescence microscopy (Keyence Corp., Osaka, Japan). Following microscopic analysis, cells were detached using TripLE, transferred to FACS tubes and CellROX™ Orange intensity was analyzed on BD AccuriC6 flow cytometer.

Iodo-BODIPY Irradiation Assay

BV-2 cells were treated for 18 hours with 5 μg/ml LPS in DMEM with 5% FBS to induce lipid droplet formation. Controls received vehicle solution (PBS) only. Cells were washed twice in PBS and incubated in 3.8 μM iodo-BODIPY (see FIG. 8, Panel A; iodo-BODIPY was kindly provided by Prof. Carolyn Bertozzi, Chemistry department, Stanford University) in PBS for 30 min in the incubator (5% CO₂, 37° C.). Cells were washed twice in PBS and then irradiated under visible light (green LED; 9 W, 150 mA, 2000 lux) for 3 hours at RT. Non-irradiated cells were kept at RT for 3 hours without irradiation.

CalceinAM Live Cell Staining

To assess cell viability after photoirradiation, irradiated and non-irradiated BV-2 cells were stained with 1 μM Calcein AM (Invitrogen) in PBS for 15 min at 37° C. Cells were then examined by fluorescence microscopy (Keyence Corp., Osaka, Japan) and the percentage of green, Calcein⁺BV-2 cells (live cells) of total methyl-di-iodo BODIPY⁺ cells (red, lipid droplet containing cells) was calculated.

CRISPR-Cas9 Screen

The 10-sgRNA-per-gene CRISPR/Cas9 deletion library was synthesized, cloned and infected into Cas9-expressing BV-2 cells as previously described⁴. Briefly, about 30 million BV-2 cells stably expressing EF1-alpha-Cas9-Blast were infected with the 10 guide/gene sgRNA sub-library (see Supplementary Table 2 for gene list) at an MOI<1. Infected cells underwent puromycin selection (1.5 μg/ml) for 7 days after which point puromycin was removed and cells were resuspended in normal growth medium (DMEM+10% FBS) without puromycin. After selection, sgRNA infection was confirmed by flow cytometry, which indicated >90% of cells expressed the mCherry reporter. Cells were cultured at 1000× coverage (about 1000 cells containing each sgRNA) throughout the screen. BV-2 cells were treated for 18 hours with 5 μg/ml LPS in DMEM with 5% FBS to induce lipid droplet formation, and the photoirradiation assay was performed as described above (see Methyl-di-iodo BODIPY irradiation assay). Non-irradiated, LPS-treated BV-2 cells were used as controls. After irradiation, cells were washed twice in medium and put back in the incubator. LPS-treatment and photoirradiation were performed three times, with 24 hours recovery time between each irradiation and the next LPS treatment. At the end of the screen genomic DNA was extracted for all experimental conditions using a QIAGEN Blood Midi Kit. Deep sequencing of sgRNA sequences on an Illumina Nextseq was used to monitor library composition. Guide composition was analyzed and compared to the plasmid library between conditions using castle version 1.0⁵ available at https://bitbucket.org/dmorgens/castle. Genes with FDR<50% were considered as significant hits.

Generation of Single CRISPR-Cas9 Knockout BV-2 Cells

Lentivirus production and infection was performed as previously described⁴. Briefly, HEK293T cells were transfected with packaging plasmids and sgRNA-containing plasmids. Supernatant was harvested at 48 and 72 hours and concentrated with Lenti-X solution (Clontech). BV-2 cells stably expressing Cas9 under blasticidin (1 μg/ml) were infected with lentivirus containing sgRNA plasmids under puromycin selection for 24 hours. Puromycin selection was started 48 hours after infection and maintained for 7 days. GRN knockout BV-2 cells were sub-cloned to maintain a monoclonal knockout population. SLC33A1 and VPS35 single-knockout cell lines were assayed as polyclonal populations.

Cytokine Assay

Primary microglia from hippocampi of 3-month (young) and 20-month-old (aged) wild type mice were isolated as described above (see Microglia Isolation, hippocampi from 3 mice were pooled per age group). CD11b⁺CD45^{lo} primary microglia from young mice, and CD11b⁺CD45^{lo} primary microglia sorted for BODIPY^{lo} and BODIPY^{hi} cells from aged mice were sorted into 5% FBS-containing microglial culture medium (DMEM/F12, 1% Pen/Strep; 2 mM glutamine; 5 ug/mL N-acetyl cysteine; 5 ug/mL insulin; 100 ug/mL apo-transferrin; 100 ng/mL sodium selenite; 2 ng/mL human TGF-2; 100 ng/mL murine IL-34; 1.5 ug/mL ovine wool cholesterol; 10 ug/mL heparin sulfate)s. Cells were seeded into 96 well plates at 5000 cells per well in 100 μl microglia culture medium with 5% FBS and incubated for 30 min in the incubator (37° C., 5% CO₂). Next, cells were treated with 100 ng/ml LPS or PBS for 8 hours, and supernatant was collected and secreted signaling proteins were measured in culture supernatants using the ‘Mouse Cytokine/Chemokine Array 31-Plex’ from Eve Technologies (Canada).

NAD/NADH Assay

Primary microglia from 3-month (young) and 20-month-old (aged) wild type mice were isolated as described above (see Microglia Isolation; hippocampi from 3 mice were pooled per group), and CD11b⁺CD45^{lo} primary microglia from young mice, and CD11b⁺CD45^{lo} primary microglia sorted for BODIPY^{lo} and BODIPY^{hi} cells from aged mice were sorted into 5% FBS-containing microglial culture medium (see Cytokine assay). Cells were seeded into 96 well white-walled tissue culture plates at 5000 cells per well in 50 μl microglial culture medium with 5% FBS and incubated for 30 min in the incubator (37° C., 5% CO₂). The NAD/NADH-Glo™ assay (Promega) was performed according to manufacturer's instructions. Cell lysates were incubated for 2 hours at RT and luminescence was recorded using a luminometer (Lmax, Molecular Devices).

Statistical Analysis

Data collection was randomized for all experiments. Experimenters were blinded for imaging and data analysis. Statistical analyses were performed using the GraphPad Prism 5.0 software (GraphPad Software), and R DESeq2 package was used for RNA-Seq analysis. Data were tested for normality using the Kolmogorov-Smirnov or the Shapiro-Wilk test, and equality of variance was confirmed using the F-test. Means between two groups were compared by the two-tailed unpaired Student's t-test or, in case of non-gaussian distribution, by using the two-tailed Mann-Whitney U-test. Data from multiple groups were analyzed by one-way ANOVA and two-way ANOVA, followed by Tukey's post hoc tests. Detailed information on sample size, numbers of replicates, and statistical test used for each experiment is provided in the Figure legends.

### FIGURE LEGENDS

FIG. 1 Microglia in the aged brain accumulate lipid droplets.

a, Electron microscopy of microglia from young and aged mice. b, Hippocampus from aged mice stained for BODIPY⁺ (lipid droplets) and TMEM119⁺ (microglia). Right panel shows 3D reconstruction of BODIPY⁺/TMEM119⁺ microglia. Arrows indicate lipid droplets. c-e, Quantification of BODIPY⁺ lipid droplet numbers (c), percent BODIPY⁺/TMEM119⁺ cells (d), and average BODIPY⁺ lipid droplet size (e) in the hippocampus (dentate gyrus). n=6 mice per group. f, Confocal images of Plin2⁺ (lipid droplets) and lba1⁺ (microglia) in the human hippocampus of a 22-year-old and 67-year-old individual. Arrow indicates Plin2⁺lba1⁺ cell. g,h, Representative images (g) and quantification (h) of CARS⁺ signal (2845 cm⁻¹) in TMEM119+ microglia in the hippocampus of young and aged mice. n=5 mice per group. i,j Experimental schematic for lipidomics analysis of lipid droplets isolated from whole hippocampus (upper panel) and from FACS-sorted microglia (lower panel) from 20-month old mice (i), and pie charts showing the composition of lipid droplets (j). n=4 mice per group. Statistical tests: unpaired Student's t-test. Error bars represent mean±SD. **P<0.01, ***P<0.001. n, nucleus; LD, lipid droplet; Ly, Lysosome; TAG, triacylglycerol; DAG, diacylglycerol; PC, phosphatidylcholine; CE, cholesteryl ester. Young=3-month-old mice; Aged=20 month-old mice.

FIG. 2 Lipid droplet accumulating microglia are abundant in the hippocampus but rare in other brain regions of aged mice. Related to FIG. 1

a-d, Representative confocal images of the cortex (a), thalamus (b), corpus callosum (c) and hippocampal dentate gyrus (d) from 20-month old mice stained for BODIPY⁺ (lipid droplets) and lba1⁺ (microglia). Scale bar: 20 pm. Arrows point towards BODIPY⁺ lipid droplets.

FIG. 3 RNA-Seq of LD-low and LD-high microglia from aged mice reveals transcriptional changes linked to phagocytosis and ROS production.

a, Flow sorting scheme for isolation of BODIPY^{lo} (=LD-low) and BODIPY^{hi} (=LD-high) CD11b⁺CD45^{lo} cells from the hippocampus of 18-month old mice. n=3 samples per group. Each sample is a pool of microglia from 3 mouse brains. b, Representative images of microglia after brain homogenization and marker staining, before (upper panel) and after (lower panel) FACS sorting. c, Volcano plot showing differentially expressed genes in LD-low versus LD-high microglia. Red circles represent genes with q<0.05. Genes involved in phagosome maturation (purple) and ROS production (blue) are highlighted. d, Heatmap showing the top 50 differentially expressed genes (ranked by p-value). e, Top canonical pathways identified by IPA that are differentially regulated between LD-low and LD-high microglia. Analysis based on top 200 genes ranked by p-value. f, IPA upstream regulator analysis of top 200 differentially expressed genes between LD-low and LD-high microglia. g, Overlap between genes changing in microglia in aging and neurodegeneration (Aging, AD, ALS, DAM, MGnD), and genes upregulated (yellow) or downregulated (blue) in LD-high microglia. Percent overlap denotes the fraction of genes in each gene list that are up- or down-regulated in LD-high microglia. h, IPA pathway analysis of genes that are significantly upregulated (yellow, left) or downregulated (blue, right) in LD-high microglia. Analysis based on top 100 down- and up-regulated genes. LD, lipid droplet.

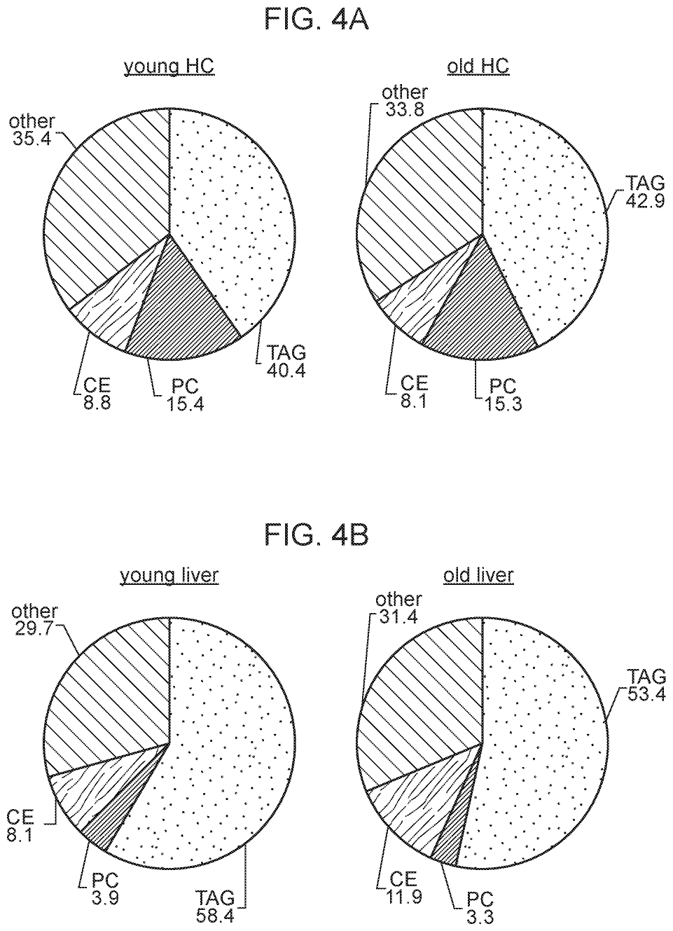

FIG. 4 Lipid droplets isolated from hippocampus of young and aged mice show almost identical lipid composition and are similar to lipid droplets isolated from the liver. Related to FIG. 1

a,b, Lipidomic analysis of lipid droplets isolated from hippocampus (a) and liver (b) from 5-month (young) and 20-month (aged) old mice. n=4 mice per group. HC, hippocampus. TAG, triacylglycerol; PC, phosphatidylcholine; CE, cholesteryl ester.

FIG. 5 LPS treatment induces lipid droplet formation in microglia.

a,b, BODIPY⁺ and lba1⁺ in the hippocampus of 3-month old mice given intraperitoneal (i.p.) injections of PBS or LPS (1 mg/kg BW) for four days. Representative confocal images of BODIPY⁺ and lba1⁺ in the hippocampus (a) and quantification of BODIPY⁺/lba1⁺ microglia (b). n=6 mice per group. c-f, BV-2 cells were treated with PBS or LPS (5 μg/ml) for 18 hours and co-treated with Triacsin C (1 μM) or saline. Representative micrographs of BODIPY⁺ staining in BV-2 cells (c) and quantification of BODIPY⁺ cells (d). e,f, Representative flow cytometry histogram (e) and quantification (f) of BODIPY fluorescence in BV-2 cells. Experiments on BV-2 cells (d,f) were performed three times in technical triplicates. Statistical tests: unpaired Student's t-test (b) and one-way ANOVA followed by Tukey's post hoc test (d,f). Error bars represent mean±SD (b). Horizontal lines in the box plots indicate medians, box limits indicate first and third quantiles, and vertical whisker lines indicate minimum and maximum values (d,f). **P<0.01, ***P<0.001. LD, lipid droplet; MFI, mean fluorescent intensity; TrC Triacsin C.

FIG. 6 LAM and lipid droplets in BV-2 cells are associated with impaired phagocytosis.

a, Pathway map of genes related to phagosome maturation that are differentially expressed in LAM (based on top 200 genes of RNA-Seq analysis, FIG. 2). Blue indicates downregulation in LAM, yellow indicates upregulation. b, Confocal images showing BODIPY⁺ (lipid droplets), CD68⁺ (endosomes/lysosomes), and lba1⁺ in the hippocampus from 20-month old mice. c, Percentage of BODIPY⁻ and BODIPY⁺lba1⁺ microglia with high levels of CD68 (CD68^{hi}). n=5 mice per group. d, 3D reconstruction of lba1⁺ microglia showing BODIPY⁺ lipid droplets closely surrounded by CD68⁺ vesicles. e, Electron microscopy image showing lysosomal accumulation in LAM from a 20-month old mouse. f,g, 250 pm organotypic brain slices from 12-month old mice were incubated for 4 hours with pHrodo red Zymosan particles. Representative confocal images of the hippocampus (f) and pie chart showing the percentages of Zymosan-containing BODIPY⁻ and BODIPY⁺lba1⁺ cells (g). P-value for Zymosan⁺ BODIPY⁻ vs Zymosan⁺ BODIPY⁺lba1⁺ cell=0.0012. n=3 mice per group. h-j, Phagocytosis of pHrodo red Zymosan in BV-2 cells treated with PBS or LPS (5 ug/ml) for 18 hours and co-treated with Triacsin C (1 μM) or saline. Representative images of Zymosan pHrodo uptake (red fluorescence) in BV-2 cells (h). Time lapse imaging and quantification of Zymosan uptake in BV-2 cells (j). High magnification confocal images of BODIPY⁺ and Zymosan⁺ in LPS-treated BV-2 cells (i). Experiments on BV-2 cells were performed three times in technical triplicates (j). Statistical tests: unpaired Student's t-test (c), two-way ANOVA followed by Tukey's post hoc test (g,j). Error bars represent mean±SD (c,g) and mean±SEM (j). *P<0.05, **P<0.01, ***P<0.001. LD, lipid droplet; Ly, Lysosomes; n, nucleus; TrC, Triacsin C.

FIG. 7 LAM and lipid droplet-rich BV-2 cells show increased ROS production and LAM secrete elevated levels of inflammatory cytokines.

a, Pathway map of genes related to ROS production that are differentially expressed in LAM (based on top 200 genes of RNA-Seq analysis, FIG. 2). Blue indicates downregulation in LAM, yellow indicates upregulation. b, Experimental schematic of ROS analysis in primary microglia from young and aged mice. c, Representative flow cytometry histogram and quantification of CellROX fluorescence in primary microglia from young and aged mice. d, Gating scheme for LD-low and LD-high microglia from aged mice. e, Histogram and quantification of CellROX fluorescence in LD-low and LD-high microglia from aged mice. f-i, CellROX fluorescence in BV-2 cells treated with PBS or LPS (5 ug/ml) for 18 hours, co-treated with Triacsin C (1 μM) or saline. f, Representative images of CellROX⁺ signal in BV-2 cells. g, Confocal images showing BODIPY⁺ and CellROX⁺ in LPS treated BV-2 cells. h,i Flow cytometry histogram (h) and quantification (i) of CellROX fluorescence in BV-2 cells. j,k Acutely isolated LD-low (BODIPY^{lo}) and LD-high (BODIPY^{hi}) primary microglia from aged mice were treated with LPS (100 ng/ml) for 8 hours, and cytokine concentrations in the medium were measured using multiplex array. Heatmap showing changes in cytokine secretion under baseline conditions and after LPS treatment (j) and individual dot plots of selected cytokines (k). Experiments were performed three (BV-2 cells) or two (primary cells) times in technical triplicates. Primary cells were isolated from 3 mice per group per experiment. Statistical tests: unpaired Student's t-test (c,e), one-way ANOVA (i) and two-way ANOVA (k) followed by Tukey's post hoc test. Horizontal lines in the box plots indicate medians, box limits indicate first and third quantiles, and vertical whisker lines indicate minimum and maximum values. *P<0.05, **P<0.01, ***P<0.001. LD, lipid droplet; MFI, mean fluorescent intensity; TrC, Triacsin C. Young=3-month-old mice; Aged=20 month-old mice.

FIG. 8 CRISPR-Cas9 screen identifies genetic regulators of lipid droplet formation.

a, Structure of BODIPY 493/503 and of iodo-BODIPY. b,c, Calcein⁺ signal in BV-2 cells that were treated with 5 μg/ml LPS for 18 hours, stained with iBP, and exposed to photoirradiation for 3 hours. Representative micrographs (b) and quantification (c) of Calcein⁺ (live cells) and iBP⁺ (lipid-droplet containing cells) labelling in control (non-irradiated) and irradiated BV-2 cells. d, Experimental schematic for pooled CRISPR-Cas9 screen to identify regulators of lipid droplet formation in LPS-treated BV-2 cells. e, Volcano plot showing hits for genetic regulators of lipid droplet formation from the CRISPR-Cas9 knockout screen. Dashed line represents P-value <0.05 cutoff. Positive CasTLE effect represents genes targeted by sgRNAs that were enriched in lipid droplet-negative cells; negative CasTLE effect represents genes targeted by sgRNAs that were under-represented in lipid droplet-negative cells. Genes previously associated with neurodegeneration are highlighted. f-j, Single CRISPR-Cas9 knockout BV-2 cell lines of selected screen hits (SNX17^{-/-}, GRN^{-/-}, SLC33A1^{-/-}, VPS35^{-/-}). Cas9-expressing BV-2 cells were used as control (CTR). Representative micrographs of BODIPY+ staining in PBS or LPS treated cells (f) and quantification of the percentage of BODIPY⁺ cells (g). Quantification of CellROX MFI in PBS or LPS treated cells (h). i,j, Time lapse imaging and quantification of Zymosan uptake in cells treated with PBS (i) or LPS (j). k-m, BODIPY⁺ and lba1⁺ expression in the hippocampus of 9-month old WT mice and age-matched GRN^{-/-} mice. Representative confocal images (k), and quantification of BODIPY⁺/lba1⁺ microglia (I) and of lipid droplet numbers per lba1⁺ microglia (m). n=5 mice per group. Experiments on BV-2 cells (c,g-j) were performed three times in technical triplicates. Statistical tests: unpaired Student's t-test (c,l,m), two-way ANOVA (g,h,i,j) followed by Tukey's post hoc test. Error bars represent mean±SD (I) and mean±SEM (i,j). Horizontal lines in the box plots indicate medians, box limits indicate first and third quantiles, and vertical whisker lines indicate minimum and maximum values. *P<0.05, **P<0.01, ***P<0.001. LD, lipid droplet; mBP, methyl-diiodinated BODIPY; MFI, mean fluorescent intensity.

FIG. 9 LAM have a unique transcriptional signature that does not overlap with published gene expression profiles of microglia in aging and neurodegeneration. Related to FIG. 3

a-e, Expression plots comparing RNA-Seq data of LAM (see FIG. 3) with published RNA-Seq data of microglia in aging (a), AD (b), ALS (c), disease-associated microglia (DAM) (d) and neurodegenerative microglia (MGnD) (e). Data are expressed as signed fdr, i.e the product of log 2 FC and log 10 fdr. f, Paired dot plots showing FPKM values of LD-low and LD-high microglia for ApoE (P=0.423) and Trem2 (P=0.038). Dotted lines connect LD-low and LD-high microglia sorted from the same samples. LD, lipid droplet.

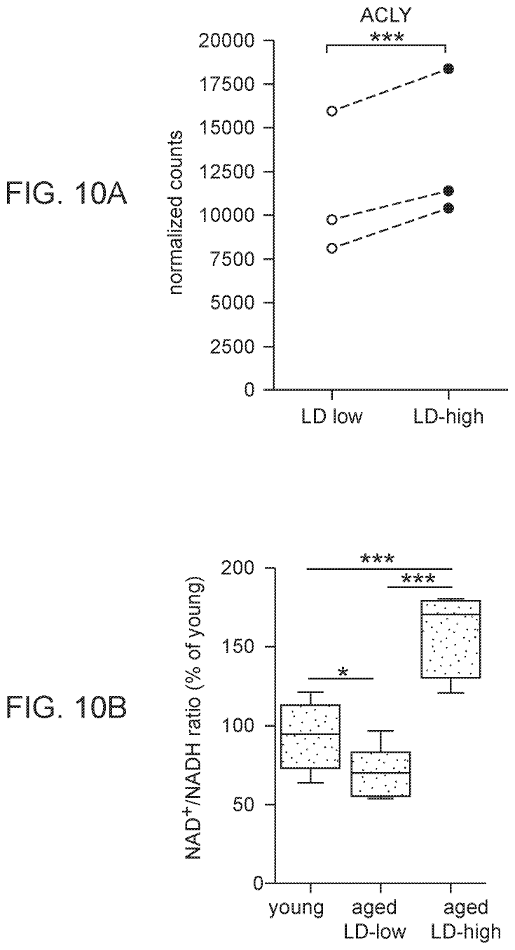

FIG. 10 LAM show signs of metabolic alterations. Related to FIG. 3

a, Paired dot plot showing FPKM values of LD-low and LD-high microglia for ACLY (data obtained from RNA-Seq analysis, see FIG. 3). Dotted lines connect LD-low and LD-high microglia sorted from the same samples. b, NAD colorimetric assay showing the NAD⁺/NADH ratio of primary hippocampal microglia from 3-month old mice (young) and of LD-low and LD-high primary microglia from 20-month old mice. Experiments were performed two times in technical triplicates. n=3 mice per group per experiment. Statistical tests: one-way ANOVA (b) followed by Tukey's post hoc test. Horizontal lines in the box plots indicate medians, box limits indicate first and third quantiles, and vertical whisker lines indicate minimum and maximum values. *P<0.05, ***P<0.001.

### REFERENCES

1 Davalos, D. et al. ATP mediates rapid microglial response to local brain injury in vivo. Nat Neurosci 8, 752-758, doi:10.1038/nn1472 (2005).
2 Nimmerjahn, A., Kirchhoff, F. & Helmchen, F. Resting microglial cells are highly dynamic surveillants of brain parenchyma in vivo. Science 308, 1314-1318, doi:10.1126/science.1110647 (2005).
3 Aguzzi, A., Barres, B. A. & Bennett, M. L. Microglia: scapegoat, saboteur, or something else? Science 339, 156-161, doi:10.1126/science.1227901 (2013).
4 Crotti, A. & Ransohoff, R. M. Microglial Physiology and Pathophysiology: Insights from Genome-wide Transcriptional Profiling. Immunity 44, 505-515, doi:10.1016/j.immuni.2016.02.013 (2016).
5 Safaiyan, S. et al. Age-related myelin degradation burdens the clearance function of microglia during aging. Nat Neurosci 19, 995-998, doi:10.1038/nn.4325 (2016).
6 Tremblay, M. E., Zettel, M. L., Ison, J. R., Allen, P. D. & Majewska, A. K. Effects of aging and sensory loss on glial cells in mouse visual and auditory cortices. Glia 60, 541-558, doi:10.1002/glia.22287 (2012).
7 Hefendehl, J. K. et al. Homeostatic and injury-induced microglia behavior in the aging brain. Aging Cell13, 60-69, doi:10.1111/ace1.12149 (2014).
8 Wilkinson, B. L. & Landreth, G. E. The microglial NADPH oxidase complex as a source of oxidative stress in Alzheimer's disease. J Neuroinflammation 3, 30, doi:10.1186/1742-2094-3-30 (2006).
9 Marschallinger, J. et al. Structural and functional rejuvenation of the aged brain by an approved anti-asthmatic drug. Nat Commun 6, 8466, doi:10.1038/ncomms9466 (2015).
10 Mrdjen, D. et al. High-Dimensional Single-Cell Mapping of Central Nervous System Immune Cells Reveals Distinct Myeloid Subsets in Health, Aging, and Disease. Immunity 48, 380-395 e386, doi:10.1016/j.immuni.2018.01.011 (2018).
11 Krasemann, S. et al. The TREM2-APOE Pathway Drives the Transcriptional Phenotype of Dysfunctional Microglia in Neurodegenerative Diseases. Immunity 47, 566-581 e569, doi:10.1016/j.immuni.2017.08.008 (2017).
12 Keren-Shaul, H. et al. A Unique Microglia Type Associated with Restricting Development of Alzheimer's Disease. Cell 169, 1276-1290 e1217, doi:10.1016/j.cell.2017.05.018 (2017).
13 Olah, M. et al. A transcriptomic atlas of aged human microglia. Nat Commun 9 , 539, doi:10.1038/s41467-018-02926-5 (2018).
14 Hammond, T. R. et al. Complex cell-state changes revealed by single cell RNA sequencing of 76,149 microglia throughout the mouse lifespan and in the injured brain. BioRxiv, doi:doi: http://dx.doi.org/10.1101/406140 (2018).
15 Li, Q. et al. Developmental heterogeneity of microglia and brain myeloid cells revealed by deep single-cell RNA sequencing. BioRxiv, doi:http://dx.doi.org/10.1101/406363 (2018).
16 Foley, P. Lipids in Alzheimer's disease: A century-old story. Biochim Biophys Acta 1801, 750-753, doi:10.1016/j.bbalip.2010.05.004 (2010).
17 Fowler, S. D., Mayer, E. P. & Greenspan, P. Foam cells and atherogenesis. Ann NY Acad Sci 454, 79-90 (1985).
18 Fowler, S., Shio, H. & Haley, N. J. Characterization of lipid-laden aortic cells from cholesterol-fed rabbits. IV. Investigation of macrophage-like properties of aortic cell populations. Lab Invest 41, 372-378 (1979).
19 Bozza, P. T. & Viola, J. P. Lipid droplets in inflammation and cancer. Prostaglandins Leukot Essent Fatty Acids 82, 243-250, doi:10.1016/j.plefa.2010.02.005 (2010).
20 den Brok, M. H., Raaijmakers, T. K., Collado-Camps, E. & Adema, G. J. Lipid Droplets as Immune Modulators in Myeloid Cells. Trends Immunol 39, 380-392, doi:10.1016/j.it.2018.01.012 (2018).
21 Yuan, Y., Li, P. & Ye, J. Lipid homeostasis and the formation of macrophage-derived foam cells in atherosclerosis. Protein Cell 3, 173-181, doi:10.1007/s13238-012-2025-6 (2012).
22 Bozza, P. T., Magalhaes, K. G. & Weller, P. F. Leukocyte lipid bodies—Biogenesis and functions in inflammation. Biochim Biophys Acta 1791, 540-551, doi:10.1016/j.bbalip.2009.01.005 (2009).
23 Vieira-de-Abreu, A. et al. Cross-talk between macrophage migration inhibitory factor and eotaxin in allergic eosinophil activation forms leukotriene C(4)-synthesizing lipid bodies. Am J Respir Cell Mol Biol 44, 509-516, doi:10.1165/rcmb.2010-00040C (2011).
24 Melo, R. C. et al. Lipid bodies in inflammatory cells: structure, function, and current imaging techniques. J Histochem Cytochem 59, 540-556, doi:10.1369/0022155411404073 (2011).
25 Farese, R. V., Jr. & Walther, T. C. Lipid droplets finally get a little R-E-S-P-E-C-T. Cell 139, 855-860, doi:10.1016/j.cell.2009.11.005 (2009).
26 Childs, B. G. et al. Senescent intimal foam cells are deleterious at all stages of atherosclerosis. Science 354, 472-477, doi:10.1126/science.aaf6659 (2016).
27 Castejon, O. J., Castellano, A., Arismendi, G. J. & Medina, Z. The inflammatory reaction in human traumatic oedematous cerebral cortex. J Submicrosc Cytol Pathol 37, 43-52 (2005).
28 Lee, S. C., Moore, G. R., Golenwsky, G. & Raine, C. S. Multiple sclerosis: a role for astroglia in active demyelination suggested by class II MHC expression and ultrastructural study. J Neuropathol Exp Neurol 49, 122-136 (1990).
29 Sturrock, R. R. Microglia in the human embryonic optic nerve. J Anat 139 (Pt 1), 81-91 (1984).
30 Liu, L., MacKenzie, K. R., Putluri, N., Maletic-Savatic, M. & Bellen, H. J. The Glia-Neuron Lactate Shuttle and Elevated ROS Promote Lipid Synthesis in Neurons and Lipid Droplet Accumulation in Glia via APOE/D. Cell Metab 26, 719-737 e716, doi:10.1016/j.cmet.2017.08.024 (2017).
31 Liu, L. et al. Glial lipid droplets and ROS induced by mitochondrial defects promote neurodegeneration. Cell 160, 177-190, doi:10.1016/j.cell.2014.12.019 (2015).
32 Shimabukuro, M. K. et al. Lipid-laden cells differentially distributed in the aging brain are functionally active and correspond to distinct phenotypes. Sci Rep 6, 23795, doi:10.1038/5rep23795 (2016).
33 Chang, P. K., Khatchadourian, A., McKinney, R. A. & Maysinger, D. Docosahexaenoic acid (DHA): a modulator of microglia activity and dendritic spine morphology. J Neuroinflammation 12, 34, doi:10.1186/s12974-015-0244-5 (2015).
34 Gasparovic, C. et al. Magnetic resonance lipid signals in rat brain after experimental stroke correlate with neutral lipid accumulation. Neurosci Lett 301, 87-90 (2001).
35 Khatchadourian, A., Bourque, S. D., Richard, V. R., Titorenko, V. I. & Maysinger, D. Dynamics and regulation of lipid droplet formation in lipopolysaccharide (LPS)-stimulated microglia. Biochim Biophys Acta 1821, 607-617, doi:10.1016/j.bbalip.2012.01.007 (2012).
36 Daemen, S., van Zandvoort, M., Parekh, S. H. & Hesselink, M. K. C. Microscopy tools for the investigation of intracellular lipid storage and dynamics. Mol Metab 5, 153-163, doi:10.1016/j.molmet.2015.12.005 (2016).
37 Harris, L. A., Skinner, J. R. & Wolins, N. E. Imaging of neutral lipids and neutral lipid associated proteins. Methods Cell Biol 116, 213-226, doi:10.1016/B978-O-12-408051-5.00011-5 (2013).
38 Yu, Y., Ramachandran, P. V. & Wang, M. C. Shedding new light on lipid functions with CARS and SRS microscopy. Biochim Biophys Acta 1841, 1120-1129, doi:10.1016/j.bbalip.2014.02.003 (2014).
39 Bennett, M. L. et al. New tools for studying microglia in the mouse and human CNS. Proc Natl Acad Sci USA 113, E1738-1746, doi:10.1073/pnas.1525528113 (2016).
40 Holtman, I. R. et al. Induction of a common microglia gene expression signature by aging and neurodegenerative conditions: a co-expression meta-analysis. Acta Neuropathol Commun 3, 31, doi:10.1186/s40478-015-0203-5 (2015).
41 Chiu, I. M. et al. A neurodegeneration-specific gene-expression signature of acutely isolated microglia from an amyotrophic lateral sclerosis mouse model. Cell Rep 4, 385-401, doi:10.1016/j.celrep.2013.06.018 (2013).
42 Wang, Y. et al. TREM2 lipid sensing sustains the microglial response in an Alzheimer's disease model. Cell 160, 1061-1071, doi:10.1016/j.cell.2015.01.049 (2015).
43 Deczkowska, A. et al. Disease-Associated Microglia: A Universal Immune Sensor of Neurodegeneration. Cell 173, 1073-1081, doi:10.1016/j.cell.2018.05.003 (2018).
44 Carmona, S. et al. The role of TREM2 in Alzheimer's disease and other neurodegenerative disorders. Lancet Neurol 17, 721-730, doi:10.1016/51474-4422(18)30232-1 (2018).
45 Zhou, Y., Ulland, T. K. & Colonna, M. TREM2-Dependent Effects on Microglia in Alzheimer's Disease. Front Aging Neurosci 10, 202, doi:10.3389/fnagi.2018.00202 (2018).
46 Chen, Z. et al. Lipopolysaccharide-induced microglial activation and neuroprotection against experimental brain injury is independent of hematogenous TLR4. J Neurosci 32, 11706-11715, doi:10.1523/JNEUROSCI.0730-12.2012 (2012).
47 Namatame, I., Tomoda, H., Arai, H., Inoue, K. & Omura, S. Complete inhibition of mouse macrophage-derived foam cell formation by triacsin C. J Biochem 125, 319-327 (1999).
48 Mosher, K. I. & Wyss-Coray, T. Microglial dysfunction in brain aging and Alzheimer's disease. Biochem Pharmacol 88, 594-604, doi:10.1016/j.bcp.2014.01.008 (2014).
49 Kawabe, K., Takano, K., Moriyama, M. & Nakamura, Y. Lipopolysaccharide-Stimulated Transglutaminase 2 Expression Enhances Endocytosis Activity in the Mouse Microglial Cell Line BV-2. Neuroimmunomodulation 22, 243-249, doi:10.1159/000365484 (2015).
50 von Bernhardi, R., Eugenin-von Bernhardi, L. & Eugenin, J. Microglial cell dysregulation in brain aging and neurodegeneration. Front Aging Neurosci 7, 124, doi:10.3389/fnagi.2015.00124 (2015).
51 Wong, W. T. Microglial aging in the healthy CNS: phenotypes, drivers, and rejuvenation. Front Cell Neurosci 7, 22, doi:10.3389/fncel.2013.00022 (2013).
52 Reis, K., Halldin, J., Fernaeus, S., Pettersson, C. & Land, T. NADPH oxidase inhibitor diphenyliodonium abolishes lipopolysaccharide-induced down-regulation of transferrin receptor expression in N2a and BV-2 cells. J Neurosci Res 84, 1047-1052, doi:10.1002/jnr.21005 (2006).
53 Morgens, D. W. et al. Genome-scale measurement of off-target activity using Cas9 toxicity in high-throughput screens. Nat Commun 8, 15178, doi:10.1038/ncomms15178 (2017).
54 Kamkaew, A. et al. BODIPY dyes in photodynamic therapy. Chem Soc Rev 42, 77-88, doi:10.1039/c2cs35216 h (2013).
55 Bonardi, L., Ulrich, G. & Ziessel, R. Tailoring the properties of boron-dipyrromethene dyes with acetylenic functions at the 2,6,8 and 4-B substitution positions. Org Lett 10, 2183-2186, doi:10.1021/ol800560b (2008).
56 Lee, J. et al. Adaptor protein sorting nexin 17 regulates amyloid precursor protein trafficking and processing in the early endosomes. J Biol Chem 283, 11501-11508, doi:10.1074/jbc.M800642200 (2008).
57 Liu, P. et al. S113R mutation in SLC33A1 leads to neurodegeneration and augmented BMP signaling in a mouse model. Dis Model Mech 10, 53-62, doi:10.1242/dmm.026880 (2017).
58 Huey, E. D. et al. Characteristics of frontotemporal dementia patients with a Progranulin mutation. Ann Neurol 60, 374-380, doi:10.1002/ana.20969 (2006).
59 Tsika, E. et al. Parkinson's disease-linked mutations in VPS35 induce dopaminergic neurodegeneration. Hum Mol Genet 23, 4621-4638, doi:10.1093/hmg/ddu178 (2014).
60 Yin, F. et al. Behavioral deficits and progressive neuropathology in progranulin-deficient mice: a mouse model of frontotemporal dementia. FASEB J 24, 4639-4647, doi:10.1096/fj.10-161471 (2010).
61 Marschallinger, J., Mosher, K. I. & Wyss-Coray, T. in Handbook of Immunosenescence: Basic Understanding and Clinical Implications (eds Tamas Fulop, Claudio Franceschi, Katsuiku Hirokawa, & Graham Pawelec) 1-15 (Springer International Publishing, 2017).
62 Gubern, A. et al. JNK and ceramide kinase govern the biogenesis of lipid droplets through activation of group IVA phospholipase A2. J Biol Chem 284, 32359-32369, doi:10.1074/jbc.M109.061515 (2009).
63 Hu, X., Xu, B. & Ge, W. The Role of Lipid Bodies in the Microglial Aging Process and Related Diseases. Neurochem Res 42, 3140-3148, doi:10.1007/s11064-017-2351-4 (2017).
64 Rambold, A. S., Cohen, S. & Lippincott-Schwartz, J. Fatty acid trafficking in starved cells: regulation by lipid droplet lipolysis, autophagy, and mitochondrial fusion dynamics. Dev Cell 32, 678-692, doi:10.1016/j.devcel.2015.01.029 (2015).
65 Lalancette-Hebert, M., Moquin, A., Choi, A. O., Kriz, J. & Maysinger, D. Lipopolysaccharide-QD micelles induce marked induction of TLR2 and lipid droplet accumulation in olfactory bulb microglia. Mol Pharm 7, 1183-1194, doi:10.1021/mp1000372 (2010).
66 Everts, B. et al. TLR-driven early glycolytic reprogramming via the kinases TBK1-IKKvarepsilon supports the anabolic demands of dendritic cell activation. Nat Immunol 15, 323-332, doi:10.1038/ni.2833 (2014).
67 Churchward, M. A., Tchir, D. R. & Todd, K. G. Microglial Function during Glucose Deprivation: Inflammatory and Neuropsychiatric Implications. Mol Neurobiol 55, 1477-1487, doi:10.1007/s12035-017-0422-9 (2018).
68 Currie, E., Schulze, A., Zechner, R., Walther, T. C. & Farese, R. V., Jr. Cellular fatty acid metabolism and cancer. Cell Metab 18, 153-161, doi:10.1016/j.cmet.2013.05.017 (2013).
69 Burke, A. C. & Huff, M. W. ATP-citrate lyase: genetics, molecular biology and therapeutic target for dyslipidemia. Curr Opin Lipidol 28, 193-200, doi:10.1097/MOL.0000000000000390 (2017).
70 Cantuti-Castelvetri, L. et al. Defective cholesterol clearance limits remyelination in the aged central nervous system. Science 359, 684-688, doi:10.1126/science.aan4183 (2018).
71 Lee, S. J., Zhang, J., Choi, A. M. & Kim, H. P. Mitochondrial dysfunction induces formation of lipid droplets as a generalized response to stress. Oxid Med Cell Longev 2013, 327167, doi:10.1155/2013/327167 (2013).
72 Korchak, H. M., Kane, L. H., Rossi, M. W. & Corkey, B. E. Long chain acyl coenzyme A and signaling in neutrophils. An inhibitor of acyl coenzyme A synthetase, triacsin C, inhibits superoxide anion generation and degranulation by human neutrophils. J Biol Chem 269, 30281-30287 (1994).
73 Nadjar, A. Role of metabolic programming in the modulation of microglia phagocytosis by lipids. Prostaglandins Leukot Essent Fatty Acids 135, 63-73, doi:10.1016/j.plefa.2018.07.006 (2018).
74 Chinetti-Gbaguidi, G. et al. Human atherosclerotic plaque alternative macrophages display low cholesterol handling but high phagocytosis because of distinct activities of the PPARgamma and LXRalpha pathways. Circ Res 108, 985-995, doi:10.1161/CIRCRESAHA.110.233775 (2011).
75 Chandak, P. G. et al. Efficient phagocytosis requires triacylglycerol hydrolysis by adipose triglyceride lipase. J Biol Chem 285, 20192-20201, doi:10.1074/jbc.M110.107854 (2010).
76 Singh, R. et al. Autophagy regulates lipid metabolism. Nature 458, 1131-1135, doi:10.1038/nature07976 (2009).
77 Peng, Y. et al. Deficient import of acetyl-CoA into the ER lumen causes neurodegeneration and propensity to infections, inflammation, and cancer. J Neurosci 34, 6772-6789, doi:10.1523/JNEUROSCI.0077-14.2014 (2014).
78 Nguyen, A. D. et al. Progranulin in the hematopoietic compartment protects mice from atherosclerosis. Atherosclerosis 277, 145-154, doi:10.1016/j.atherosclerosis.2018.08.042 (2018).
79 Evers, B. M. et al. Lipidomic and Transcriptomic Basis of Lysosomal Dysfunction in Progranulin Deficiency. Cell Rep 20, 2565-2574, doi:10.1016/j.celrep.2017.08.056 (2017).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of the present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating a neurodegenerative disorder in a subject in need thereof, the method comprising administering to the subject an effective amount of an agent that reduces the prevalence of lipid droplet accumulating microglia, thereby treating the neurodegenerative disorder.

2. The method according to claim 1, wherein the subject is a mammal.

3. The method according to claim 2, wherein the neurodegenerative disorder is selected from the group consisting of: Alzheimer's disease, Parkinson's disease, Amyotrophic Lateral Sclerosis, Huntington's disease, and frontotemporal dementia.

4. The method according to any one of claims 1 to 3, wherein the agent is a granulin (GRN) agonist, a solute carrier family 33 member 1 (SLC33A1) agonist, a vacuolar protein sorting-associated protein 35 (VPS35) agonist, or a Sorting nexin-17 (SNX17) antagonist.

5. The method according to claim 4, wherein the agent is an SLC33A1 agonist and the neurodegenerative disorder is Parkinson's disease or frontotemporal dementia.

6. The method according to claim 5, wherein the SLC33A1 agonist is an SLC33A1 peptide, an SLC33A1 encoding nucleic acid, or a small molecule SLC33A1 agonist.

7. The method according to claim 4, wherein the agent is an VPS35 agonist and the neurodegenerative disorder is Alzheimer's disease or frontotemporal dementia.

8. The method according to claim 7, wherein the VPS35 agonist is a VPS35 peptide, a VPS35 encoding nucleic acid, or a small molecule VPS35 agonist.

9. The method according to claim 4, wherein the agent is an SNX17 antagonist and the neurodegenerative disorder is Parkinson's disease or frontotemporal dementia.

10. The method according to claim 9, wherein the SNX17 antagonist is an SNX17 antibody, an SNX17 CRISPR-based gene silencing agent, an SNX17 interfering nucleic acid, or a small molecule SNX17 antagonist.

11. The method according to any one of claims 1 to 10, wherein the subject comprises a wildtype GRN locus, a wildtype SLC33A1 locus, a wildtype VPS35 locus, a wildtype SNX17 locus, or a combination thereof.

12. The method according to any one of claims 1 to 11, wherein the method results in at least a 5% reduction in the prevalence of lipid droplet accumulating microglia.

13. The method according to any one of claims 1 to 12, wherein the method further comprises administering to the subject a general inhibitor of lipid droplet formation.

14. The method according to claim 13, wherein the general inhibitor of lipid droplet formation comprises a lipogenesis inhibitor.

15. A method of identifying a lipid droplet-associated target gene, the method comprising:
contacting a population of microglia with a plurality of CRISPR-based gene silencing agents targeting a plurality of target genes;
inducing lipid droplet formation in the contacted population of microglia;
selecting lipid droplet-deficient cells from the induced population and sequencing the selected cells; and
identifying a lipid droplet-associated target gene based on the sequencing, wherein enhanced representation of the target gene indicates a positive regulator of lipid droplet formation and reduced representation of the target gene indicates a negative regulator of lipid droplet formation.

16. The method according to claim 15, wherein the population of microglia comprise primary cells.

17. The method according to claim 15, wherein the population of microglia comprise cells of a cultured cell line.

18. The method according to any one of claims 15 to 17, wherein the population of microglia comprises lipid droplet accumulating microglia.

19. The method according to any one of claims 15 to 18, wherein the population of microglia expresses Cas9.

20. The method according to any one of claims 15 to 19, wherein inducing lipid droplet formation comprises contacting the population of microglia with lipopolysaccharide.

21. The method according to any one of claims 15 to 20, wherein the selecting comprises lipid-based photoablation.

22. A method of treating a neurodegenerative disorder in a subject in need thereof, the method comprising administering to the subject an effective amount of an antagonist of the positive regulator of lipid droplet formation identified according to any one of claims 15 to 21.

23. A method of treating a neurodegenerative disorder in a subject in need thereof, the method comprising administering to the subject an effective amount of an agonist of the negative regulator of lipid droplet formation identified according to any one of claims 15 to 21.


SEQUENCE LISTING (6 sequences)

SEQ ID NO: 1 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - protein, 71 aa, Homo sapiens
MAITAAHGASTAVQTGDPASKDQVTTPWVPSSALIVSSNARTSPRAVLWSMAPGGAAPCPRLPAVKTGCTA

SEQ ID NO: 3 - protein, 549 aa, Homo sapiens
MSPTISHKDSSRQRRPGNFSHSLDMKSGPLPPGGWDDSHLDSAGREGDREALLGDTGTGDFLKAPQSFRAELSSILLLLFLYVLQGIPLGLAGSIPLILQSKNVSYTDQAFFSFVFWPFSLKLLWAPLVDAVYVKNFGRRKSWLVPTQYILGLFMIYLSTQVDRLLGNTDDRTPDVIALTVAFFLFEFLAATQDIAVDGWALTMLSRENVGYASTCNSVGQTAGYFLGNVLFLALESADFCNKYLRFQPQPRGIVTLSDFLFFWGTVFLITTTLVALLKKENEVSVVKEETQGITDTYKLLFAIIKMPAVLTFCLLILTAKIGFSAADAVTGLKLVEEGVPKEHLALLAVPMVPLQIILPLIISKYTAGPQPLNTFYKAMPYRLLLGLEYALLVWWTPKVEHQGGFPIYYYIVVLLSYALHQVTVYSMYVSIMAFNAKVSDPLIGGTYMTLLNTVSNLGGNWPSTVALWLVDPLTVKECVGASNQNCRTPDAVELCKKLGGSCVTALDGYYVESIICVFIGFGWWFFLGPKFKKLQDEGSSSWKCKRNN

SEQ ID NO: 4 - protein, 731 aa, Homo sapiens
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFY

SEQ ID NO: 5 - protein, 470 aa, Homo sapiens
MHFSIPETESRSGDSGGSAYVAYNIHVNGVLHCRVRYSQLLGLHEQLRKEYGANVLPAFPPKKLFSLTPAEVEQRREQLEKYMQAVRQDPLLGSSETFNSFLRRAQQETQQVPTEEVSLEVLLSNGQKVLVNVLTSDQTEDVLEAVAAKLDLPDDLIGYFSLFLVREKEDGAFSFVRKLQEFELPYVSVTSLRSQEYKIVLRKSYWDSAYDDDVMENRVGLNLLYAQTVSDIERGWILVTKEQHRQLKSLQEKVSKKEFLRLAQTLRHYGYLRFDACVADFPEKDCPVVVSAGNSELSLQLRLPGQQLREGSFRVTRMRCWRVTSSVPLPSGSTSSPGRGRGEVRLELAFEYLMSKDRLQWVTITSPQAIMMSICLQSMVDELMVKKSGGSIRKMLRRRVGGTLRRSDSQQAVKSPPLLESPDATRESMVKLSSKLSAVSLRGIGSPSTDASASDVHGNFAFEGIGDEDL

SEQ ID NO: 6 - protein, 445 aa, Homo sapiens
MHFSIPETESRSGDSGGSAYVLRKEYGANVLPAFPPKKLFSLTPAEVEQRREQLEKYMQAVRQDPLLGSSETFNSFLRRAQQETQQVPTEEVSLEVLLSNGQKVLVNVLTSDQTEDVLEAVAAKLDLPDDLIGYFSLFLVREKEDGAFSFVRKLQEFELPYVSVTSLRSQEYKIVLRKSYWDSAYDDDVMENRVGLNLLYAQTVSDIERGWILVTKEQHRQLKSLQEKVSKKEFLRLAQTLRHYGYLRFDACVADFPEKDCPVVVSAGNSELSLQLRLPGQQLREGSFRVTRMRCWRVTSSVPLPSGSTSSPGRGRGEVRLELAFEYLMSKDRLQWVTITSPQAIMMSICLQSMVDELMVKKSGGSIRKMLRRRVGGTLRRSDSQQAVKSPPLLESPDATRESMVKLSSKLSAVSLRGIGSPSTDASASDVHGNFAFEGIGDEDL